(19) United States
(12) Patent Application Publication
(10) Pub. No.: US 2025/0009863 A1
(43) Pub. Date: Jan. 9, 2025

(54) LYME DISEASE RNA VACCINE

(71) Applicant: SANOFI, Paris (FR)

(72) Inventors: Vincent PAVOT, Paris (FR); Yves GIRERD-CHAMBAZ, Paris (FR); Céline CIVAT, Paris (FR); Corinne PION, Paris (FR)

(21) Appl. No.: 18/741,976

(22) Filed: Jun. 13, 2024

Related U.S. Application Data
(63) Continuation of application No. PCT/EP2022/086341, filed on Dec. 16, 2022.

(30) Foreign Application Priority Data
Dec. 17, 2021 (EP) 21315283.8
Dec. 23, 2021 (EP) 21315291.1

(51) Int. Cl. A61K 39/02; A61K 9/50; A61K 39/00; A61P 37/04

(52) U.S. Cl. CPC A61K 39/0225; A61K 9/5015; A61P 37/04; A61K 2039/53; A61K 2039/55; A61K 2039/552

(57) ABSTRACT

The present disclosure provides a Lyme disease vaccine, comprising a messenger RNA (mRNA) comprising an open reading frame (ORF) encoding at least one antigenic polypeptide derived from at least one bacteria of the genus Borrelia, and methods of eliciting an immune response by administering said vaccine.

### RELATED APPLICATIONS

This application is a continuation of International Patent Application No. PCT/EP2022/086341, filed Dec. 16, 2022, which claims priority to European Patent Application Nos. 21315283.8, filed Dec. 17, 2021, and 21315291.1, filed Dec. 23, 2021, the contents of each incorporated by reference in their entirety for all purposes.

### SEQUENCE LISTING

The instant application contains a Sequence Listing which has been submitted electronically in XML format and is hereby incorporated by reference in its entirety. Said XML file, created on Jun. 12, 2024, is named 754677_SA9-321PCCON_ST26.xml and is 35,658 bytes in size.

### BACKGROUND OF THE DISCLOSURE

Lyme borreliosis (i.e., Lyme disease) is a zoonotic disease caused by some bacterial species in the genus Borrelia and is transmitted to humans and other mammals by the bite of an infected Ixodes spp. tick. Lyme disease is a global public health problem, with cases reported from temperate climates across Europe, North America, and Asia. Outer surface protein A (OspA) is an abundant immunogenic lipoprotein of Borrelia. There are at least seven different serotypes (serotypes 1-7) of OspA that are found in Borrelia worldwide, and different genospecies of Borrelia that can cause Lyme borreliosis exist worldwide. Further, localized ranges of ticks that harbor Borrelia means that an OspA serotype that is associated with Lyme disease in patients in one geographic region might not be associated with Lyme disease in patients in another geographic region.

RNA-based vaccines (e.g., mRNA vaccines) have recently emerged as an additional vaccine type with a rapid, safe, and cost-effective production process, in particular against viral pathogens. mRNA vaccines against severe acute respiratory syndrome coronavirus 2 (SARS-CoV-2) mostly use the spike viral protein, as antigen. Often combined with a delivery vehicle, such as a lipid nanoparticle (LNP), COVID-19 mRNA vaccines may achieve high efficacy. With the dearth of effective Lyme disease vaccines available, there exists a need for RNA-based Lyme disease vaccines that elicit strong immune responses for potent neutralization of a Lyme disease infection.

### BRIEF SUMMARY OF THE DISCLOSURE

In one aspect, the disclosure provides a Lyme disease vaccine, comprising a messenger RNA (mRNA) comprising an open reading frame (ORF) encoding at least one antigenic polypeptide derived from at least one bacteria of the genus Borrelia.

In certain embodiments, the at least one bacteria is selected from the species B. burgdorferi, afzelii, garinii, bavariensis, mayonii, spielmanii, lusitaniae, bissettii and/or valaisiana, or any strain or isolate thereof.

In certain embodiments, the at least one antigenic polypeptide comprises at least one lipoprotein of Borrelia.

In certain embodiments, the at least one lipoprotein is OspA or a fragment or variant thereof. Preferably, the fragment or variant comprises at least 5 amino acids.

In certain embodiments, the at least one OspA is derived from OspA serotype (ST) 1, 2, 3, 4, 5, 6, and/or 7.

In certain embodiments, the at least one OspA serotype 1-7 is from Borrelia burgdorferi strain B31 of Serotype 1, Borrelia afzelii strain PKO of Serotype 2, Borrelia garinii strain PBr of Serotype 3, Borrelia bavariensis of Serotype 4, Borrelia garinii of Serotype 5, Borrelia garinii of serotype 6, or Borrelia garinii of Serotype 7.

In certain embodiments, the at least one OspA polypeptide comprises an amino acid sequence with at least 85% identity to any one of SEQ ID NOs: 1-7.

In certain embodiments, the mRNA of the Lyme disease vaccine disclosed herein, comprises a nucleotide sequence that is at least 85% identical to any one of SEQ ID NOs: 10-13 and 16-19.

In certain embodiments, the mRNA of the Lyme disease vaccine disclosed herein, encodes at least two different OspA serotypes or fragments or variants thereof. Preferably, each fragment or variant comprises at least 5 amino acids.

In certain embodiments, the OspA of one serotype or fragment or variant thereof is fused to a OspA of a different serotype or fragment or variant thereof.

In certain embodiments, the fused OspA of different serotypes or fragments or variants thereof are separated by a linker sequence.

In certain embodiments, the linker sequence is derived from P66.

In certain embodiments, the linker sequence comprises an amino acid sequence with at least 85% identity to SEQ ID NO: 8.

In certain embodiments, the linker sequence comprises an amino acid sequence with at least 85% identity to SEQ ID NO: 9.

In certain embodiments, the mRNA is non-replicating mRNA.

In certain embodiments, the mRNA is self-replicating or trans-replicating mRNA.

In certain embodiments, the mRNA comprises at least one chemical modification.

In certain embodiments, the chemical modification is selected from the group consisting of pseudouridine, N1-methylpseudouridine, 2-thiouridine, 4′-thiouridine, 5-methylcytidine, 2-thio-I-methyl-1-deaza-pseudouridine, 2-thio-I-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-I-methyl-pseudouridine, 4-thio-pseudouridine, 5-aza-uridine, dihydropseudouridine, 5-methyluridine, 5-methyluridine, 5-methoxyuridine, and 2′-O-methyl uridine.

In certain embodiments, the chemical modification is selected from the group consisting of pseudouridine, N1-methylpseudouridine, 5-methylcytidine, 5-methoxyuridine, and a combination thereof. In certain embodiments, the chemical modification is N1-methylpseudouridine.

In certain embodiments, the mRNA is formulated in non-viral delivery systems.

In certain embodiments, the mRNA is formulated in a lipid nanoparticle (LNP).

In certain embodiments, the LNP comprises at least one cationic lipid.

In certain embodiments, the cationic lipid is biodegradable. In certain embodiments, the cationic lipid is not biodegradable.

In certain embodiments, the cationic lipid is cleavable. In certain embodiments, the cationic lipid is not cleavable.

In certain embodiments, the cationic lipid is selected from the group consisting of ML7/OF-02; cKK-E10; GL-HEPES-E3-E10-DS-3-E18-1; GL-HEPES-E3-E12-DS-4-E10; GL-HEPES-E3-E12-DS-3-E14; 9-heptadecanyl 8-{(2-hydroxyethyl) [6-oxo-6-(undecyloxy)hexyl]amino}octanoate (SM-102); and (4-hydroxybutyl)azanediyl]di(hexane-6,1-diyl) bis(2-hexyldecanoate) (ALC-0315).

In certain embodiments, the cationic lipid is cKK-E10.

In certain embodiments, the LNP further comprises a polyethylene glycol (PEG) conjugated (PEGylated) lipid, a cholesterol-based lipid, and a helper lipid.

In certain embodiments, the LNP comprises: a cationic lipid at a molar ratio between 35% and 55%; a polyethylene glycol (PEG) conjugated (PEGylated) lipid at a molar ratio between 0.25% and 2.75%, a cholesterol-based lipid at a molar ratio between 20% and 45%, and a helper lipid at a molar ratio of between 5% and 35%, wherein all of the molar ratios are relative to the total lipid content of the LNP.

In certain embodiments, the LNP comprises: a cationic lipid at a molar ratio of 40%, a PEGylated lipid at a molar ratio of 1.5%, a cholesterol-based lipid at a molar ratio of 28.5%, and a helper lipid at a molar ratio of 30%.

In certain embodiments, the PEGylated lipid is dimyristoyl-PEG2000 (DMG-PEG2000) or 2-[(polyethylene glycol)-2000]-N,N-ditetradecylacetamide (ALC-0159).

In certain embodiments, the cholesterol-based lipid is cholesterol.

In certain embodiments, the helper lipid is 1,2-dioleoyl-SN-glycero-3-phosphoethanolamine (DOPE) or 1,2-distearoyl-sn-glycero-3-phosphocholine (DSPC).

In some embodiments, the LNP comprises: cKK-E10 at a molar ratio of 40%, DMG-PEG2000 at a molar ratio of 1.5%, cholesterol at a molar ratio of 28.5%, and DOPE at a molar ratio of 30%.

In certain embodiments, the LNP has an average diameter of 30-200 nm. In certain embodiments, the LNP has an average diameter of 80-150 nm.

In certain embodiments, the mRNA of the Lyme disease vaccine disclosed herein, comprises at least one 5′ untranslated region (5′ UTR), at least one 3′ untranslated region (3′ UTR), and at least one polyadenylation (polyA) sequence.

In certain embodiments, the mRNA comprises at least of the following structural elements:
(i) a 5′ cap with the following structure:

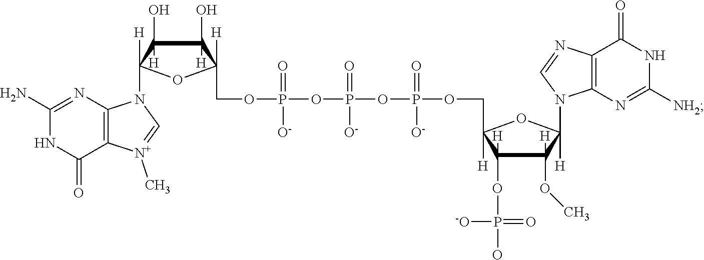

(ii) a 5′ untranslated region (5′ UTR) having the nucleic acid sequence of SEQ ID NO: 14;
(iii) at least one OspA open reading frame having any one of the nucleic acid sequences of SEQ ID NOs: 16-19;
(iv) a 3′ untranslated region (3′ UTR) having the nucleic acid sequence of SEQ ID NO: 15; and
(v) a polyA tail.

In one aspect, the disclosure provides a Lyme disease vaccine, comprising a messenger RNA (mRNA) comprising an open reading frame (ORF) encoding at least one antigenic polypeptide derived from at least one bacteria of the genus Borrelia, wherein the mRNA comprises at least of the following structural elements:
(i) a 5′ cap with the following structure:

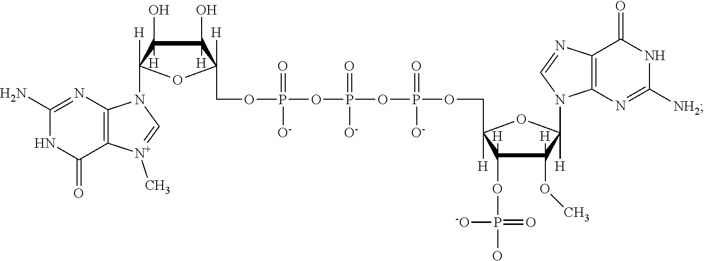

(ii) a 5′ untranslated region (5′ UTR) having the nucleic acid sequence of SEQ ID NO: 14;
(iii) at least one OspA open reading frame having any one of the nucleic acid sequences of SEQ ID NOs: 16-19;
(iv) a 3′ untranslated region (3′ UTR) having the nucleic acid sequence of SEQ ID NO: 15; and
(v) a polyA tail;
wherein the mRNA is formulated in a lipid nanoparticle (LNP) comprising: cKK-E10 at a molar ratio of 40%, DMG-PEG2000 at a molar ratio of 1.5%, cholesterol at a molar ratio of 28.5%, and DOPE at a molar ratio of 30%.

In another aspect, the Lyme disease vaccine disclosed herein is for use in eliciting an immune response, preferably a humoral immune response, and/or in treating or preventing Lyme disease in a subject in need thereof.

In another aspect, the disclosure provides a method of eliciting an immune response, preferably a humoral immune response, and/or of treating or preventing Lyme disease in a subject in need thereof, comprising administering to the subject, optionally intramuscularly, intranasally, intravenously, subcutaneously, or intradermally, an effective amount of the Lyme disease vaccine disclosed herein.

In another aspect, the disclosure provides a use of the Lyme disease vaccine disclosed herein for the manufacture of a medicament for use in eliciting an immune response, preferably a humoral immune response, and/or in treating or preventing Lyme disease, in a subject in need thereof.

In certain embodiments, the subject has a higher serum concentration of antibodies against OspA after administration of the Lyme disease vaccine, relative to a subject that is administered a Lyme disease vaccine comprising an OspA recombinant protein vaccine.

In certain embodiments, the subject is a mammal, optionally a human, a dog, a cat, a llama, a bovine, a sheep, a goat, a horse, a rodent, a mouse, a rat, a rabbit, a monkey, a primate or a pig. In particularly exemplary embodiments, the subject is a human.

### BRIEF DESCRIPTION OF THE DRAWINGS/FIGURES

The foregoing and other features and advantages of the present disclosure will be more fully understood from the following detailed description of illustrative embodiments taken in conjunction with the accompanying drawings.

FIG. 1A-FIG. 1B depict IgG titer values in mice, from an anti-OspA ST1 IgG ELISA, post-dose 1 (day 20) (FIG. 1A) and post-dose 2 (day 35) (FIG. 1B). Dotted line=limit of quantification.

FIG. 2A-FIG. 2B depicts IgG titer values in mice, from an anti-OspA ST2 IgG ELISA, post-dose 1 (day 20) (FIG. 2A) and post-dose 2 (day 35) (FIG. 2B). Dotted line=limit of quantification.

### DETAILED DESCRIPTION OF THE DISCLOSURE

The present disclosure is directed to, inter alia, novel RNA (e.g., mRNA) compositions encoding an antigenic polypeptide derived from Borrelia, such as an OspA protein, and methods of vaccination with the same. In particular, the disclosure relates to mRNA encoding an OspA protein formulated in a non-viral delivery system, in particular a lipid nanoparticle (LNP).

### I. Definitions

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of, cell and tissue culture, molecular biology, virology, immunology, microbiology, genetics, analytical chemistry, synthetic organic chemistry, medicinal and pharmaceutical chemistry, and protein and nucleic acid chemistry and hybridization described herein are those well-known and commonly used in the art. Enzymatic reactions and purification techniques are performed according to manufacturer's specifications, as commonly accomplished in the art or as described herein. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety. Although a number of documents are cited herein, this citation does not constitute an admission that any of these documents forms part of the common general knowledge in the art.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, may provide one of skill with a general dictionary of many of the terms used in this disclosure.

Units, prefixes, and symbols are denoted in their International System of Units (SI) accepted form. Numeric ranges are inclusive of the numbers defining the range. Unless otherwise indicated, amino acid sequences are written left to right in amino to carboxy orientation. The headings provided herein are not limitations of the various aspects of the disclosure. Accordingly, the terms defined immediately below are more fully defined by reference to the specification in its entirety.

The term “approximately” or “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower). In some embodiments, the term indicates deviation from the indicated numerical value by ±10%, ±5%, ±4%, ±3%, ±2%, ±1%, ±0.9%, ±0.8%, ±0.7%, ±0.6%, ±0.5%, ±0.4%, ±0.3%, ±0.2%, ±0.1%, ±0.05%, or ±0.01%. In some embodiments, “about” indicates deviation from the indicated numerical value by ±10%. In some embodiments, “about” indicates deviation from the indicated numerical value by ±5%. In some embodiments, “about” indicates deviation from the indicated numerical value by ±4%. In some embodiments, “about” indicates deviation from the indicated numerical value by ±3%. In some embodiments, “about” indicates deviation from the indicated numerical value by ±2%. In some embodiments, “about” indicates deviation from the indicated numerical value by ±1%. In some embodiments, “about” indicates deviation from the indicated numerical value by ±0.9%. In some embodiments, “about” indicates deviation from the indicated numerical value by ±0.8%. In some embodiments, “about” indicates deviation from the indicated numerical value by ±0.7%. In some embodiments, “about” indicates deviation from the indicated numerical value by ±0.6%. In some embodiments, “about” indicates deviation from the indicated numerical value by ±0.5%. In some embodiments, “about” indicates deviation from the indicated numerical value by ±0.4%. In some embodiments, “about” indicates deviation from the indicated numerical value by ±0.3%. In some embodiments, “about” indicates deviation from the indicated numerical value by ±0.1%. In some embodiments, “about” indicates deviation from the indicated numerical value by ±0.05%. In some embodiments, “about” indicates deviation from the indicated numerical value by ±0.01%.

As used herein, the term “messenger RNA” or “mRNA” refers to a polynucleotide that encodes at least one polypeptide. mRNA as used herein encompasses both modified and unmodified RNA. mRNA may contain one or more coding and non-coding regions. A coding region is alternatively referred to as an open reading frame (ORF). Non-coding regions in mRNA include the 5′ cap, 5′ untranslated region (UTR), 3′ UTR, and a polyA tail. mRNA can be purified from natural sources, produced using recombinant expression systems (e.g., in vitro transcription) and optionally purified, or chemically synthesized.

As used herein, the term “open reading frame”, “ORF”, or “coding region” refers to a polynucleotide sequence beginning with a start codon (e.g. ATG) and ending with a stop codon (e.g. TAA, TAG or TGA), without any other stop codon in between, and that encodes a protein (e.g. an antigenic polypeptide derived from a bacteria of the genus Borrelia).

Also included in the present disclosure are fragments or variants of polypeptides, and any combination thereof. The term “fragment” or “variant” when referring to the OspA polypeptide molecules of the present disclosure include any polypeptides which retain at least some of the properties (e.g., specific antigenic property of the polypeptide or the ability of polypeptide to contribute to the induction of antibody binding) of the reference polypeptide. Fragments of polypeptides include N-terminally and/or C-terminally truncated fragments, e.g. C-terminal fragments and N-terminal fragments, as well as deletion fragments but do not include the naturally occurring full-length polypeptide (or mature polypeptide). A deletion fragment refers to a polypeptide with 1 or more internal amino acids deleted from the full-length polypeptide. Variants of polypeptides include fragments as described above, and also polypeptides with altered amino acid sequences due to amino acid substitutions, deletions, or insertions. Variants can be naturally or non-naturally occurring. Non-naturally occurring variants can be produced using art-known mutagenesis techniques. Variant polypeptides can comprise conservative or non-conservative amino acid substitutions, deletions or additions. Such variations (i.e. truncations and/or amino acid substitutions, deletions, or insertions) may occur either on the amino acid level or correspondingly on the nucleic acid level.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, if an amino acid in a polypeptide is replaced with another amino acid from the same side chain family, the substitution is considered to be conservative. In another embodiment, a string of amino acids can be conservatively replaced with a structurally similar string that differs in order and/or composition of side chain family members.

The term “linked” as used herein refers to a first amino acid sequence or nucleotide sequence covalently or non-covalently joined to a second amino acid sequence or nucleotide sequence, respectively. The first amino acid or nucleotide sequence can be directly joined or juxtaposed to the second amino acid or nucleotide sequence or alternatively an intervening sequence can covalently join the first sequence to the second sequence. The term “linked” means not only a fusion of a first amino acid sequence to a second amino acid sequence at the C-terminus or the N-terminus, but also includes insertion of the whole first amino acid sequence (or the second amino acid sequence) into any two amino acids in the second amino acid sequence (or the first amino acid sequence, respectively). In one embodiment, the first amino acid sequence can be linked to a second amino acid sequence by a peptide bond or a linker. The first nucleotide sequence can be linked to a second nucleotide sequence by a phosphodiester bond or a linker. The linker can be a peptide or a polypeptide (for polypeptide chains) or a nucleotide or a nucleotide chain (for nucleotide chains) or any chemical moiety (for both polypeptide and polynucleotide chains). The term “linked” is also indicated by a hyphen (-).

As used herein, the term “immune response” refers to a response of a cell of the immune system, such as a B cell, T cell, dendritic cell, macrophage or polymorphonucleocyte, to a stimulus such as an antigen or vaccine. An immune response can include any cell of the body involved in a host defense response, including for example, an epithelial cell that secretes an interferon or a cytokine. An immune response includes, but is not limited to, an innate and/or adaptive immune response.

As used herein, a “protective immune response” refers to an immune response that protects a subject from infection (e.g., prevents infection or prevents the development of disease associated with infection). Methods of measuring immune responses are well known in the art and include, for example, by measuring proliferation and/or activity of lymphocytes (such as B or T cells), secretion of cytokines or chemokines, inflammation, antibody production and the like.

As used herein, an “antibody response” is an immune response in which antibodies are produced.

As used herein, an “antigen” refers to an agent that elicits an immune response, and/or an agent that is bound by a T cell receptor (e.g., when presented by an MHC molecule) or to an antibody (e.g., produced by a B cell) when exposed or administered to an organism. In some embodiments, an antigen elicits a humoral response (e.g., including production of antigen-specific antibodies) in an organism. Alternatively, or additionally, in some embodiments, an antigen elicits a cellular response (e.g., involving T-cells whose receptors specifically interact with the antigen) in an organism. A particular antigen may elicit an immune response in one or several members of a target organism (e.g., mice, rabbits, primates, humans), but not in all members of the target organism species. In some embodiments, an antigen elicits an immune response in at least about 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the members of a target organism species. In some embodiments, an antigen binds to an antibody and/or T cell receptor, and may or may not induce a particular physiological response in an organism. In some embodiments, for example, an antigen may bind to an antibody and/or to a T cell receptor in vitro, whether or not such an interaction occurs in vivo. In some embodiments, an antigen reacts with the products of specific humoral or cellular immunity. Antigens include the OspA polypeptides (e.g., OspA ST1 and ST2) encoded by the mRNA as described herein.

As used herein, an “adjuvant” refers to a substance or vehicle that enhances the immune response to an antigen. Adjuvants can include, without limitation, a suspension of minerals (e.g., alum, aluminum hydroxide, or phosphate) on which antigen is adsorbed; a water-in-oil or oil-in-water emulsion in which antigen solution is emulsified in mineral oil or in water (e.g., Freund's incomplete adjuvant). Sometimes killed mycobacteria is included (e.g., Freund's complete adjuvant) to further enhance antigenicity. Immuno-stimulatory oligonucleotides (e.g., a CpG motif) can also be used as adjuvants (for example, see U.S. Pat. Nos. 6,194,388; 6,207,646; 6,214,806; 6,218,371; 6,239,116; 6,339,068; 6,406,705; and 6,429,199). Adjuvants can also include biological molecules, such as Toll-Like Receptor (TLR) agonists and costimulatory molecules.

As used herein, an “antigenic OspA polypeptide” refers to a polypeptide comprising all or part of an OspA amino acid sequence of sufficient length that the molecule is antigenic with respect to Lyme disease and the OspA polypeptide.

As used herein, a “subject” refers to any member of the animal kingdom. In some embodiments, “subject” refers to humans. In some embodiments, “subject” refers to non-human animals. In certain embodiments, the non-human subject is a mammal, e.g., a rodent, a mouse, a rat, a rabbit, a monkey, a llama, a horse, a dog, a cat, a bovine, a sheep, a goat, a primate, or a pig). In some embodiments, wherein the subject is a human, the terms “individual” or “patient” are used and are intended to be interchangeable with “subject”.

As used herein, the terms “prevent”, “preventing”, “prevention” or “prophylaxis” (and grammatical variants thereof) refer to partially or completely inhibiting the onset of one or more symptoms or features of a particular infection, disease, disorder, and/or condition.

As used herein, the terms “treat”, “treating”, “treatment”, “therapy” or “therapeutic” (and grammatical variants thereof) refer to partially or completely alleviating, ameliorating, improving, relieving, inhibiting progression of, and/or reducing severity of one or more symptoms or features of an infection, disease, disorder, and/or condition.

As used herein, the term “effective amount” refers to an amount (e.g. of a nucleic acid or composition) sufficient to effect beneficial or desired results. An effective amount can be administered in one or more administrations, applications or dosages, and is not intended to be limited to a particular formulation or administration route.

The term “effective amount” includes e.g. “therapeutically effective amount” and/or “prophylactically effective amount”.

The phrase “therapeutically effective amount” as used herein refers to an amount (e.g. of a nucleic acid or composition) which is effective for producing some desired therapeutic effects in the treatment of an infection, disease, disorder and/or condition at a reasonable benefit/risk ratio applicable to any medical treatment.

The phrase “prophylactically effective amount” as used herein refers to an amount (e.g. of a nucleic acid or composition) which is effective for producing some desired prophylactic effects in the prevention of an infection, disease, disorder and/or condition at a reasonable benefit/risk ratio applicable to any medical treatment.

As used herein, the term “vaccination” or “vaccinate” refers to the administration of a composition intended to generate an immune response, for example to a disease-causing agent. Vaccination can be administered before, during, and/or after exposure to a disease-causing agent, and/or to the development of one or more symptoms, and in some embodiments, before, during, and/or shortly after exposure to the agent. In some embodiments, vaccination includes multiple administrations, appropriately spaced in time, of a vaccine composition.

The disclosure describes nucleic acid sequences (e.g., DNA and RNA sequences) and amino acid sequences having a certain degree of identity to a given nucleic acid sequence or amino acid sequence, respectively (a reference sequence).

“Sequence identity” between two nucleic acid sequences indicates the percentage of nucleotides that are identical between the sequences. “Sequence identity” between two amino acid sequences indicates the percentage of amino acids that are identical between the sequences.

The terms “% identical”, “% identity” or similar terms are intended to refer, in particular, to the percentage of nucleotides or amino acids which are identical in an optimal alignment between the sequences to be compared. Said percentage is purely statistical, and the differences between the two sequences may be but are not necessarily randomly distributed over the entire length of the sequences to be compared. Comparisons of two sequences are usually carried out by comparing said sequences, after optimal alignment, with respect to a segment or “window of comparison”, in order to identify local regions of corresponding sequences. The optimal alignment for a comparison may be carried out manually or with the aid of the local homology algorithm by Smith and Waterman, 1981, Ads App. Math. 2, 482, with the aid of the local homology algorithm by Needleman and Wunsch, 1970, J. Mol. Biol. 48, 443, with the aid of the similarity search algorithm by Pearson and Lipman, 1988, Proc. Natl Acad. Sci. USA 88, 2444, or with the aid of computer programs using said algorithms (GAP, BESTFIT, FASTA, BLAST P, BLAST N and TFASTA in Wisconsin Genetics Software Package, Genetics Computer Group, 575 Science Drive, Madison, Wis.).

Percentage identity is obtained by determining the number of identical positions at which the sequences to be compared correspond, dividing this number by the number of positions compared (e.g., the number of positions in the reference sequence) and multiplying this result by 100.

In some embodiments, the degree of identity is given for a region which is at least about 50%, at least about 60%, at least about 70%, at least about 80%, at least about 90% or about 100% of the entire length of the reference sequence. For example, if the reference nucleic acid sequence consists of 200 nucleotides, the degree of identity is given for at least about 100, at least about 120, at least about 140, at least about 160, at least about 180, or about 200 nucleotides, in some embodiments in continuous nucleotides. In some embodiments, the degree of identity is given for the entire length of the reference sequence.

Nucleic acid sequences or amino acid sequences having a particular degree of identity to a given nucleic acid sequence or amino acid sequence, respectively, may have at least one functional property of said given sequence, e.g., and in some instances, are functionally equivalent to said given sequence. In some embodiments, a nucleic acid sequence or amino acid sequence having a particular degree of identity to a given nucleic acid sequence or amino acid sequence is functionally equivalent to said given sequence.

As used herein, the term “kit” refers to a packaged set of related components, such as one or more compounds or compositions and one or more related materials such as solvents, solutions, buffers, instructions, or desiccants.

### II. RNA

The Lyme disease vaccines of the present disclosure comprise at least one ribonucleic acid (RNA) comprising an ORF encoding an antigenic polypeptide derived from Borrelia, such as an OspA protein antigen (e.g., OspA ST1 or ST2). In certain embodiments, the RNA is a messenger RNA (mRNA) comprising an open reading frame encoding an OspA protein antigen. In certain embodiments, the RNA (e.g., mRNA) further comprises at least one 5′ UTR, 3′ UTR, a polyA tail, and/or a 5′ cap.

### II. A. 5′ Cap

An mRNA 5′ cap can provide resistance to nucleases found in most eukaryotic cells and promote translation efficiency. Several types of 5′ caps are known. A 7-methylguanosine cap (also referred to as “m⁷G” or “Cap-0”), comprises a guanosine that is linked through a 5′-5′-triphosphate bond to the first transcribed nucleotide.

A 5′ cap is typically added as follows: first, an RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5′5′5 triphosphate linkage; and the 7-nitrogen of guanine is then methylated by a methyltransferase. Examples of cap structures include, but are not limited to, m7G(5′)ppp, (5′(A,G(5′)ppp(5′)A and G(5′)ppp(5′)G. Additional cap structures are described in U.S. Publication No. US 2016/0032356 and U.S. Publication No. US 2018/0125989, which are incorporated herein by reference.

5′-capping of polynucleotides may be completed concomitantly during the in vitro-transcription reaction using the following chemical RNA cap analogs to generate the 5′-guanosine cap structure according to manufacturer protocols: 3′-O-Me-m7G(5′)ppp(5′)G (the ARCA cap); G(5′)ppp(5′)A; G(5′)ppp(5′)G; m7G(5′)ppp(5′)A; m7G(5′)ppp(5′)G; m7G(5′)ppp(5′)(2′OMeA)pG; m7G(5′)ppp(5′)(2′OMeA)pU; m7G(5′)ppp(5′)(2′OMeG)pG (New England BioLabs, Ipswich, MA; TriLink Biotechnologies). 5′-capping of modified RNA may be completed post-transcriptionally using a Vaccinia Virus Capping Enzyme to generate the Cap 0 structure: m7G(5′)ppp(5′)G. Cap 1 structure may be generated using both Vaccinia Virus Capping Enzyme and a 2′-O methyl-transferase to generate: m7G(5′)ppp(5′)G-2′-O-methyl. Cap 2 structure may be generated from the Cap 1 structure followed by the 2′-O-methylation of the 5′-antepenultimate nucleotide using a 2′-O methyl-transferase. Cap 3 structure may be generated from the Cap 2 structure followed by the 2′-O-methylation of the 5′-preantepenultimate nucleotide using a 2′-O methyl-transferase.

In certain embodiments, the mRNA of the disclosure comprises a 5′ cap selected from the group consisting of 3′-O-Me-m7G(5′)ppp(5′)G (the ARCA cap), G(5′)ppp(5′)A, G(5′)ppp(5′)G, m7G(5′)ppp(5′)A, m7G(5′)ppp(5′)G, m7G(5′)ppp(5′)(2′OMeA)pG, m7G(5′)ppp(5′)(2′OMeA)pU, and m7G(5′)ppp(5′)(2′OMeG)pG.

In certain embodiments, the mRNA of the disclosure comprises a 5′ cap of:

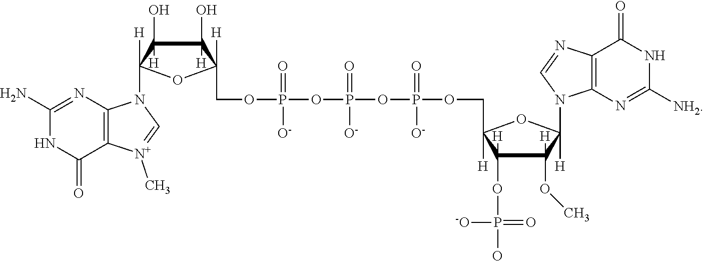

### II. B. Untranslated Region (UTR)

In some embodiments, the mRNA of the disclosure includes a 5′ and/or 3′ untranslated region (UTR). In mRNA, the 5′ UTR starts at the transcription start site and continues to the start codon but does not include the start codon. The 3′ UTR starts immediately following the stop codon and continues until the transcriptional termination signal.

In some embodiments, the mRNA disclosed herein comprise a 5′ UTR that includes one or more elements that affect an mRNA's stability or translation. In some embodiments, a 5′ UTR may be about 10 to 5,000 nucleotides in length. In some embodiments, a 5′ UTR may be about 50 to 500 nucleotides in length. In some embodiments, the 5′ UTR is at least about 10 nucleotides in length, about 20 nucleotides in length, about 30 nucleotides in length, about 40 nucleotides in length, about 50 nucleotides in length, about 100 nucleotides in length, about 150 nucleotides in length, about 200 nucleotides in length, about 250 nucleotides in length, about 300 nucleotides in length, about 350 nucleotides in length, about 400 nucleotides in length, about 450 nucleotides in length, about 500 nucleotides in length, about 550 nucleotides in length, about 600 nucleotides in length, about 650 nucleotides in length, about 700 nucleotides in length, about 750 nucleotides in length, about 800 nucleotides in length, about 850 nucleotides in length, about 900 nucleotides in length, about 950 nucleotides in length, about 1,000 nucleotides in length, about 1,500 nucleotides in length, about 2,000 nucleotides in length, about 2,500 nucleotides in length, about 3,000 nucleotides in length, about 3,500 nucleotides in length, about 4,000 nucleotides in length, about 4,500 nucleotides in length or about 5,000 nucleotides in length.

In some embodiments, the mRNA disclosed herein comprise a 3′ UTR comprising one or more of a polyadenylation signal, a binding site for proteins that affect an mRNA's stability of location in a cell, or one or more binding sites for miRNAs. In some embodiments, a 3′ UTR may be 50 to 5,000 nucleotides in length or longer. In some embodiments, a 3′ UTR may be 50 to 1,000 nucleotides in length or longer. In some embodiments, the 3′ UTR is about 50 nucleotides in length, about 100 nucleotides in length, about 150 nucleotides in length, about 200 nucleotides in length, about 250 nucleotides in length, about 300 nucleotides in length, about 350 nucleotides in length, about 400 nucleotides in length, about 450 nucleotides in length, about 500 nucleotides in length, about 550 nucleotides in length, about 600 nucleotides in length, about 650 nucleotides in length, about 700 nucleotides in length, about 750 nucleotides in length, about 800 nucleotides in length, about 850 nucleotides in length, about 900 nucleotides in length, about 950 nucleotides in length, about 1,000 nucleotides in length, about 1,500 nucleotides in length, about 2,000 nucleotides in length, about 2,500 nucleotides in length, about 3,000 nucleotides in length, about 3,500 nucleotides in length, about 4,000 nucleotides in length, about 4,500 nucleotides in length, or about 5,000 nucleotides in length. In some embodiments, the mRNA disclosed herein may comprise a 5′ or 3′ UTR that is derived from a gene distinct from the one encoded by the mRNA transcript (i.e., the UTR is a heterologous UTR).

In some embodiments, the mRNA disclosed herein may comprise a 5′ or 3′ UTR that is derived from a gene distinct from the one encoded by the mRNA transcript (i.e., the UTR is a heterologous UTR).

In certain embodiments, the 5′ and/or 3′ UTR sequences are derived from mRNA which are stable (e.g., globin, actin, GAPDH, tubulin, histone, or citric acid cycle enzymes) to increase the stability of the mRNA. For example, a 5′ UTR sequence may include a partial sequence of a CMV immediate-early 1 (IE 1) gene, or a fragment thereof to improve the nuclease resistance and/or improve the half-life of the mRNA. Also contemplated is the inclusion of a sequence encoding human growth hormone (hGH), or a fragment thereof to the 3′ end or untranslated region of the mRNA. Generally, these modifications improve the stability and/or pharmacokinetic properties (e.g., half-life) of the mRNA relative to their unmodified counterparts, and include, for example modifications made to improve such mRNA resistance to in vivo nuclease digestion.

Exemplary 5′ UTRs include a sequence derived from a CMV immediate-early 1 (IE1) gene (U.S. Publication No. 2014/0206753 and 2015/0157565, each of which is incorporated herein by reference), or the sequence GGGAUCCUACC (SEQ ID NO: 20) (U.S. Publication No. 2016/0151409, incorporated herein by reference).

In various embodiments, the 5′ UTR is derived from the 5′ UTR of a TOP gene. TOP genes are typically characterized by the presence of a 5′-terminal oligopyrimidine (TOP) tract. Furthermore, most TOP genes are characterized by growth-associated translational regulation. However, TOP genes with a tissue specific translational regulation are also known. In certain embodiments, the 5′ UTR derived from the 5′ UTR of a TOP gene lacks the 5′ TOP motif (the oligopyrimidine tract) (e.g., U.S. Publication Nos. 2017/0029847, 2016/0304883, 2016/0235864, and 2016/0166710, each of which is incorporated herein by reference).

In certain embodiments, the 5′ UTR is derived from a ribosomal protein Large 32 (L32) gene (U.S. Publication No. 2017/0029847, supra).

In certain embodiments, the 5′ UTR is derived from the 5′ UTR of an hydroxysteroid (17-b) dehydrogenase 4 gene (HSD17B4) (U.S. Publication No. 2016/0166710, supra).

In certain embodiments, the 5′ UTR is derived from the 5′ UTR of an ATP5A1 gene (U.S. Publication No. 2016/0166710, supra).

In some embodiments, an internal ribosome entry site (IRES) is used instead of a 5′ UTR.

In some embodiments, the 5′UTR comprises a nucleic acid sequence set forth in SEQ ID NO: 14. In some embodiments, the 3′UTR comprises a nucleic acid sequence set forth in SEQ ID NO: 15. The 5′ UTR and 3′UTR are described in further detail in WO2012075040, incorporated herein by reference.

### II. C. Polyadenylated Tail

As used herein, the terms “poly(A) sequence” or “poly(A) tail” or “poly(A) region” is a sequence of adenosine nucleotides at the 3′ end of the mRNA molecule. The poly(A) tail may confer stability to the mRNA and protect it from exonuclease degradation, and is also thought to enhance translation. In some embodiments, the poly(A) tail is essentially homopolymeric, e.g., a poly(A) tail of e.g., 100 adenosine nucleotides has essentially the length of 100 nucleotides. In other embodiments, the poly(A) tail may be interrupted by at least one nucleotide different from an adenosine nucleotide, e.g., a poly(A) tail of e.g., 100 adenosine nucleotides may have a length of more than 100 nucleotides (comprising 100 adenosine nucleotides and in addition said at least one nucleotide- or a stretch of nucleotides-different from an adenosine nucleotide). In certain embodiments, the poly(A) tail comprises the sequence

(SEQ ID NO: 21)
AAAAAAAAAAAAAAAAAAAAAAAAAAAAAAGCAUAUGACUAAAAAAAAA
AAAAAAAAAAAAAAAAAAAAAAAAAAAAAAAAAAAAAAAAAAAAAAAAA
AAAAAAAAAAAA.

The “poly(A) tail” as defined herein typically relates to RNA-however in the context of the disclosure, the term likewise relates to corresponding sequences in a DNA molecule (e.g., a “poly(T) sequence”).

The poly(A) tail may comprise about 10 to about 500 adenosine nucleotides, about 10 to about 200 adenosine nucleotides, about 40 to about 200 adenosine nucleotides, or about 40 to about 150 adenosine nucleotides. The length of the poly(A) tail may be at least about 10, 50, 75, 100, 150, 200, 250, 300, 350, 400, 450, or 500 adenosine nucleotides.

In some embodiments where the nucleic acid is an RNA, the poly(A) tail of the nucleic acid is obtained from a DNA template during RNA in vitro transcription. In other embodiments, the poly(A) tail is obtained in vitro by common methods of chemical synthesis without being transcribed from a DNA template. In other embodiments, poly(A) tails are generated by enzymatic poly(A)denylation of the RNA (after RNA in vitro transcription) using commercially available poly(A)denylation kits and corresponding protocols known in the art, or alternatively, by using immobilized poly(A) polymerases e.g., using methods and means as described in WO2016/174271.

The nucleic acid may comprise a poly(A) tail obtained by enzymatic polyadenylation, wherein the majority of nucleic acid molecules comprise about 100 (+/−20) to about 500 (+/−50), or about 250 (+/−20) adenosine nucleotides.

In other embodiments, the nucleic acid may comprise a poly(A) tail derived from a template DNA and may additionally comprise at least one additional poly(A) tail generated by enzymatic polyadenylation, e.g., as described in WO2016/091391.

In further embodiments, the nucleic acid comprises at least one polyadenylation signal.

In other embodiments, the nucleic acid may comprise at least one poly(C) sequence.

The term “poly(C) sequence” as used herein is intended to be a sequence of cytosine nucleotides of up to about 200 cytosine nucleotides. In some embodiments, the poly(C) sequence comprises about 10 to about 200 cytosine nucleotides, about 10 to about 100 cytosine nucleotides, about 20 to about 70 cytosine nucleotides, about 20 to about 60 cytosine nucleotides, or about 10 to about 40 cytosine nucleotides. In a particularly exemplary embodiment, the poly(C) sequence comprises about 30 cytosine nucleotides.

### II. D. Chemical Modification

The mRNA disclosed herein may be modified or unmodified. In some embodiments, the mRNA disclosed herein may contain one or more modifications that typically enhance RNA stability. Exemplary modifications include backbone modifications, sugar modifications, or base modifications. In some embodiments, the disclosed mRNA may be synthesized from naturally occurring nucleotides and/or nucleotide analogues (modified nucleotides) including, but not limited to, purines (adenine (A), guanine (G)) or pyrimidines (thymine (T), cytosine (C), uracil (U)), and as modified nucleotides analogues or derivatives of purines and pyrimidines, such as e.g. 1-methyl-adenine, 2-methyl-adenine, 2-methylthio-N-6-isopentenyl-adenine, N6-methyl-adenine, N6-isopentenyl-adenine, 2-thio-cytosine, 3-methyl-cytosine, 4-acetyl-cytosine, 5-methyl-cytosine, 2,6-diaminopurine, 1-methyl-guanine, 2-methyl-guanine, 2,2-dimethyl-guanine, 7-methyl-guanine, inosine, 1-methyl-inosine, pseudouracil (5-uracil), dihydro-uracil, 2-thio-uracil, 4-thio-uracil, 5-carboxymethylaminomethyl-2-thio-uracil, 5-(carboxyhydroxymethyl)-uracil, 5-fluoro-uracil, 5-bromo-uracil, 5-carboxymethylaminomethyl-uracil, 5-methyl-2-thio-uracil, 5-methyl-uracil, N-uracil-5-oxy acetic acid methyl ester, 5-methylaminomethyl-uracil, 5-methoxyaminomethyl-2-thio-uracil, 5′-methoxycarbonylmethyl-uracil, 5-methoxy-uracil, uracil-5-oxyacetic acid methyl ester, uracil-5-oxyacetic acid (v), 1-methyl-pseudouracil, queosine, 3-D-mannosyl-queosine, phosphoramidates, phosphorothioates, peptide nucleotides, methylphosphonates, 7-deazaguanosine, 5-methylcytosine, and inosine.

In some embodiments, the disclosed mRNA comprises at least one chemical modification including but not limited to, pseudouridine, N1-methylpseudouridine, 2-thiouridine, 4′-thiouridine, 5-methylcytosine, 2-thio-I-methyl-1-deaza-pseudouridine, 2-thio-I-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-I-methyl-pseudouridine, 4-thio-pseudouridine, 5-aza-uridine, dihydropseudouridine, 5-methyluridine, 5-methyluridine, 5-methoxyuridine, and 2′-O-methyl uridine.

In some embodiments, the chemical modification is selected from the group consisting of pseudouridine, N1-methylpseudouridine, 5-methylcytosine, 5-methoxyuridine, and a combination thereof.

In some embodiments, the chemical modification comprises N1-methylpseudouridine.

In some embodiments, at least 20%, at least 30%, at least 40%, at least 50%, at least 60%, at least 70%, at least 80%, at least 85%, at least 90%, at least 95%, or 100% of the uracil nucleotides in the mRNA are chemically modified.

In some embodiments, at least 20%, at least 30%, at least 40%, at least 50%, at least 60%, at least 70%, at least 80%, at least 85%, at least 90%, at least 95%, or 100% of the uracil nucleotides in the ORF are chemically modified.

The preparation of such analogues is known to a person skilled in the art e.g., from the U.S. Pat. Nos. 4,373,071, 4,401,796, 4,415,732, 4,458,066, 4,500,707, 4,668,777, 4,973,679, 5,047,524, 5,132,418, 5,153,319, 5,262,530, and 5,700,642.

II. E. mRNA Synthesis

The mRNAs disclosed herein may be synthesized according to any of a variety of known methods. For example, mRNAs according to the present disclosure may be synthesized via in vitro transcription (IVT). Methods for in vitro transcription are known in the art. See, e.g., Geall et al. (2013) Semin. Immunol. 25 (2): 152-159; Brunelle et al. (2013) Methods Enzymol. 530:101-14. Briefly, IVT is typically performed with a linear or circular DNA template containing a promoter, a pool of ribonucleotide triphosphates, a buffer system that may include DTT and magnesium ions, and an appropriate RNA polymerase (e.g., T3, T7 or SP6 RNA polymerase), DNase I, pyrophosphatase, and/or RNase inhibitor. The exact conditions will vary according to the specific application. The presence of these reagents is undesirable in a final mRNA product and are considered impurities or contaminants which must be purified to provide a clean and homogeneous mRNA that is suitable for therapeutic use. While mRNA provided from in vitro transcription reactions may be desirable in some embodiments, other sources of mRNA can be used according to the instant disclosure including wild-type mRNA produced from bacteria, fungi, plants, and/or animals.

In certain embodiments, the mRNA comprises at least of the following structural elements:
(i) a 5′ cap with the following structure:

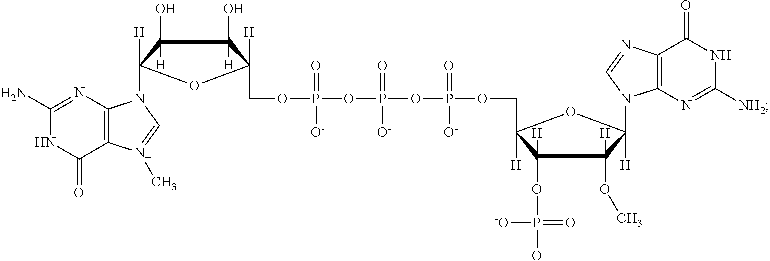

(ii) a 5′ untranslated region (5′ UTR) having the nucleic acid sequence of SEQ ID NO: 14;
(iii) at least one OspA open reading frame having any one of the nucleic acid sequences of SEQ ID NOs: 16-19;
(iv) a 3′ untranslated region (3′ UTR) having the nucleic acid sequence of SEQ ID NO: 15; and
(v) a polyA tail.

In certain embodiments, the poly(A) tail has a length of about 10 to about 500 adenosine nucleotides.

### III. OspA Protein

The causative agent of Lyme disease are bacteria of the Borrelia genus. Four species from the Borrelia genus cause most human disease: B. burgdorferi, B. afzelii, B. garinii and B. bavariensis. Each Borrelia species has surface expression of the Outer surface protein A (OspA), a useful protein target for vaccination in the treatment of Lyme disease. OspA exists in a number of serotypes, as defined by their reactivity with monoclonal antibodies against different epitopes of OspA (see Wilske et al., J Clin Microbiol 31 (2): 340-350 (1993)). These serotypes are correlated with different genospecies of Borrelia bacteria. In some embodiments, the OspA is any one of serotypes 1-7 (ST1, ST2, ST3, ST4, ST5, ST6, or ST7). In some embodiments, the OspA is from Borrelia burgdorferi, Borrelia mayonii, Borrelia afzelii, Borrelia garinii, Borrelia bavariensis, Borrelia spielmanni, Borrelia lusitaniae, Borrelia bissettii, and/or Borrelia valaisiana. In some embodiments, the OspA is Borrelia burgdorferi OspA. In some embodiments, the Borrelia can be carried by a tick of the Ixodes genus. In some embodiments, the Borrelia is Borrelia burgdorferi, Borrelia mayonii, Borrelia afzelii, Borrelia garinii, or Borrelia bavariensis.

In one aspect, the disclosure provides a Lyme disease vaccine, comprising a messenger RNA (mRNA) comprising an open reading frame (ORF) encoding at least one antigenic polypeptide derived from at least one bacteria of the genus Borrelia.

In certain embodiments, the at least one antigenic polypeptide comprises at least one lipoprotein of Borrelia.

In certain embodiments, the at least one lipoprotein is OspA or a fragment or variant thereof. Preferably, the fragment or variant comprises at least 5, 6, 7, 8, 9, 10, 11, 12, 13, 14, 15, 16, 17, 18, 19, 20, 21, 22, 23, 24, 25, 30, 35, 40, 45, 50, 60, 70, 80, 90, 100 amino acids.

In certain embodiments, the at least one OspA is derived from OspA serotype (ST) 1, 2, 3, 4, 5, 6, and/or 7.

In certain embodiments, the at least one OspA serotype 1-7 is from Borrelia burgdorferi strain B31 of Serotype 1, Borrelia afzelii strain PKO of Serotype 2, Borrelia garinii strain PBr of Serotype 3, Borrelia bavariensis of Serotype 4, Borrelia garinii of Serotype 5, Borrelia garinii of serotype 6, or Borrelia garinii of Serotype 7.

In certain embodiments, the OspA polypeptide is OspA serotype 1 (ST1). In certain embodiments, the OspA ST1 polypeptide comprises an amino acid sequence with at least 85% identity to SEQ ID NO: 1. In certain embodiments, the OspA polypeptide is OspA serotype 1 (ST1). In certain embodiments, the OspA ST1 polypeptide comprises an amino acid sequence with 85%, 86%, 87%, 88%, 89%, 90%, 91%, 92%, 93%, 94%, 95%, 96%, 97%, 98%, 99%, or 100% identity to SEQ ID NO: 1. SEQ ID NO: 1 corresponds to OspA from Borrelia burgdorferi strain B31 (Serotype 1) NCBI sequence ID WP_010890378.1, without its signal sequence and the N-terminal methionine amino acid.

In certain embodiments, the OspA ST1 polypeptide is encoded by a nucleotide sequence set forth in any one of SEQ ID NOs: 10, 11, 16, or 17.

In certain embodiments, the OspA polypeptide is OspA serotype 2 (ST2). In certain embodiments, the OspA ST2 polypeptide comprises an amino acid sequence with at least 85% identity to SEQ ID NO: 2. In certain embodiments, the OspA polypeptide is OspA serotype 2 (ST2). In certain embodiments, the OspA ST2 polypeptide comprises an amino acid sequence with 85%, 86%, 87%, 88%, 89%, 90%, 91%, 92%, 93%, 94%, 95%, 96%, 97%, 98%, 99%, or 100% identity to SEQ ID NO: 2. SEQ ID NO: 2 corresponds to OspA from Borrelia afzelii strain PKO (Serotype 2) NCBI sequence: WP_011703777.1, without its signal sequence and the N-terminal methionine amino acid.

In certain embodiments, the OspA ST2 polypeptide is encoded by a nucleotide sequence set forth in any one of SEQ ID NOs: 12, 13, 18 or 19.

In certain embodiments, the OspA polypeptide is OspA serotype 3 (ST3). In certain embodiments, the OspA ST3 polypeptide comprises an amino acid sequence with at least 85% identity to SEQ ID NO: 3. In certain embodiments, the OspA polypeptide is OspA serotype 3 (ST3). In certain embodiments, the OspA ST3 polypeptide comprises an amino acid sequence with 85%, 86%, 87%, 88%, 89%, 90%, 91%, 92%, 93%, 94%, 95%, 96%, 97%, 98%, 99%, or 100% identity to SEQ ID NO: 3. SEQ ID NO: 3 corresponds to OspA from Borrelia garinii strain PBr (Serotype 3) GenBank: CAA56549.1, without its signal sequence and the N-terminal methionine amino acid.

In certain embodiments, the OspA polypeptide is OspA serotype 4 (ST4). In certain embodiments, the OspA ST4 polypeptide comprises an amino acid sequence with at least 85% identity to SEQ ID NO: 4. In certain embodiments, the OspA polypeptide is OspA serotype 4 (ST4). In certain embodiments, the OspA ST4 polypeptide comprises an amino acid sequence with 85%, 86%, 87%, 88%, 89%, 90%, 91%, 92%, 93%, 94%, 95%, 96%, 97%, 98%, 99%, or 100% identity to SEQ ID NO: 4. SEQ ID NO: 4 corresponds to OspA from Borrelia bavariensis (Serotype 4) NCBI sequence WP_011187157.1, without its signal sequence and the N-terminal methionine amino acid.

In certain embodiments, the OspA polypeptide is OspA serotype 5 (ST5). In certain embodiments, the OspA ST5 polypeptide comprises an amino acid sequence with at least 85% identity to SEQ ID NO: 5. In certain embodiments, the OspA polypeptide is OspA serotype 5 (ST5). In certain embodiments, the OspA ST5 polypeptide comprises an amino acid sequence with 85%, 86%, 87%, 88%, 89%, 90%, 91%, 92%, 93%, 94%, 95%, 96%, 97%, 98%, 99%, or 100% identity to SEQ ID NO: 5. SEQ ID NO: 5 corresponds to OspA from Borrelia garinii (Serotype 5) GenBank CAA59727.1, without its signal sequence and the N-terminal methionine amino acid.

In certain embodiments, the OspA polypeptide is OspA serotype 6 (ST6). In certain embodiments, the OspA ST6 polypeptide comprises an amino acid sequence with at least 85% identity to SEQ ID NO: 6. In certain embodiments, the OspA polypeptide is OspA serotype 6 (ST6). In certain embodiments, the OspA ST6 polypeptide comprises an amino acid sequence with 85%, 86%, 87%, 88%, 89%, 90%, 91%, 92%, 93%, 94%, 95%, 96%, 97%, 98%, 99%, or 100% identity to SEQ ID NO: 6. SEQ ID NO: 6 corresponds to OspA from Borrelia garinii (serotype 6) GenBank: CAA45010.1, without its signal sequence and the N-terminal methionine amino acid.

In certain embodiments, the OspA polypeptide is OspA serotype 7 (ST7). In certain embodiments, the OspA ST7 polypeptide comprises an amino acid sequence with at least 85% identity to SEQ ID NO: 7. In certain embodiments, the OspA polypeptide is OspA serotype 7 (ST7). In certain embodiments, the OspA ST7 polypeptide comprises an amino acid sequence with 85%, 86%, 87%, 88%, 89%, 90%, 91%, 92%, 93%, 94%, 95%, 96%, 97%, 98%, 99%, or 100% identity to SEQ ID NO: 7. SEQ ID NO: 7 corresponds to OspA from Borrelia garinii (Serotype 7) GenBank CAA56547.1, without its signal sequence and the N-terminal methionine amino acid.

### IV. Lipid Nanoparticle (LNP)

The LNPs of the disclosure comprise four categories of lipids: (i) an ionizable lipid (e.g., cationic lipid); (ii) a PEGylated lipid; (iii) a cholesterol-based lipid (e.g., cholesterol), and (iv) a helper lipid.

### A. Ionizable Lipid

An ionizable lipid facilitates mRNA encapsulation and may be a cationic lipid. A cationic lipid affords a positively charged environment at low pH to facilitate efficient encapsulation of the negatively charged mRNA drug substance.

In some embodiments, the cationic lipid is OF-02:

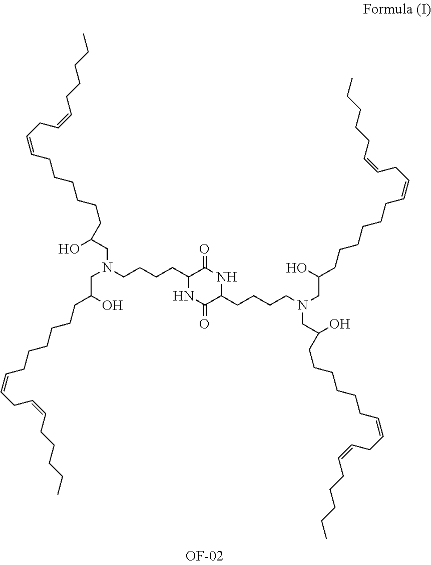 [SMILES: CCCCC/C=C\C/C=C\CCCCCCC(O)CN(CCCCC1NC(=O)C(CCCCN(CC(O)CCCCCC/C=C\C/C=C\CCCCC)CC(O)CCCCCC/C=C\C/C=C\CCCCC)NC1=O)CC(O)CCCCCC/C=C\C/C=C\CCCCC]

OF-02 is a non-degradable structural analog of OF-Deg-Lin. OF-Deg-Lin contains degradable ester linkages to attach the diketopiperazine core and the doubly-unsaturated tails, whereas OF-02 contains non-degradable 1,2-amino-alcohol linkages to attach the same diketopiperazine core and the doubly-unsaturated tails (Fenton et al., Adv Mater. (2016) 28:2939; U.S. Pat. No. 10,201,618). An exemplary LNP formulation herein, Lipid A, contains OF-2.

In some embodiments, the cationic lipid is cKK-E10 (Dong et al., PNAS (2014) 111 (11): 3955-60; U.S. Pat. No. 9,512,073):

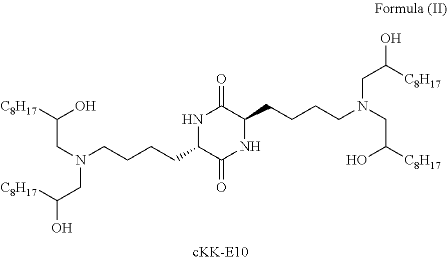

An exemplary LNP formulation herein, Lipid B, contains cKK-E10.

In some embodiments, the cationic lipid is GL-HEPES-E3-E10-DS-3-E18-1 (2-(4-(2-((3-(Bis((Z)-2-hydroxyoctadec-9-en-1-yl)amino)propyl)disulfaneyl)ethyl) piperazin-1-yl)ethyl 4-(bis(2-hydroxydecyl)amino)butanoate), which is a HEPES-based disulfide cationic lipid with a piperazine core, having the Formula III:

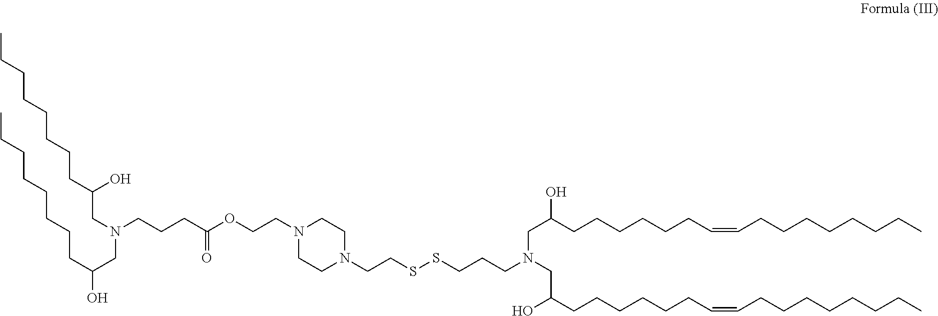 [SMILES: CCCCCCCC/C=C\CCCCCCC(O)CN(CCCSSCCN1CCN(CCOC(=O)CCCN(CC(O)CCCCCCCC)CC(O)CCCCCCCC)CC1)CC(O)CCCCCC/C=C\CCCCCCCC]

An exemplary LNP formulation herein, Lipid C, contains GL-HEPES-E3-E10-DS-3-E18-1. Lipid C has the same composition as Lipid A or Lipid B but for the difference in the cationic lipid.

In some embodiments, the cationic lipid is GL-HEPES-E3-E12-DS-4-E10 (2-(4-(2-((3-(bis(2-hydroxydecyl)amino)butyl)disulfaneyl)ethyl)piperazin-1-yl)ethyl 4-(bis(2-hydroxydodecyl)amino)butanoate), which is a HEPES-based disulfide cationic lipid with a piperazine core, having the Formula IV:

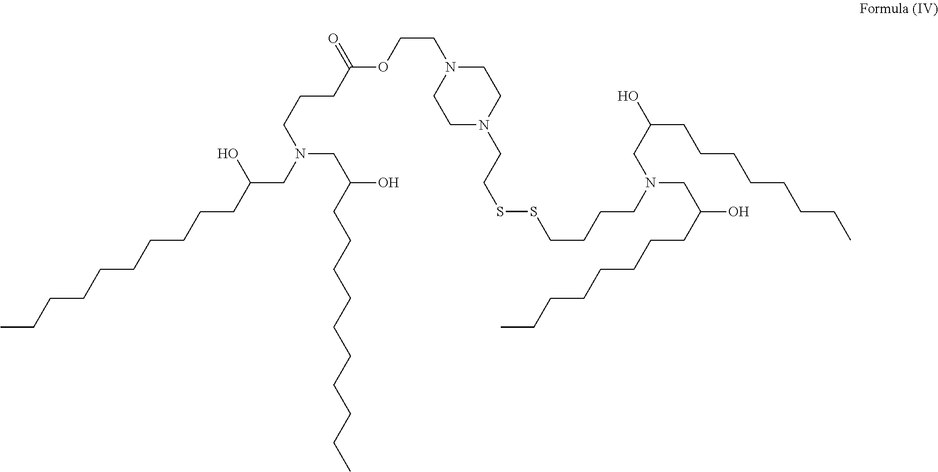 [SMILES: CCCCCCCCCCC(O)CN(CCCC(=O)OCCN1CCN(CCSSCCCCN(CC(O)CCCCCCCC)CC(O)CCCCCCCC)CC1)CC(O)CCCCCCCCCC]

An exemplary LNP formulation herein, Lipid D, contains GL-HEPES-E3-E12-DS-4-E10. Lipid D has the same composition as Lipid A or Lipid B but for the difference in the cationic lipid.

In some embodiments, the cationic lipid is GL-HEPES-E3-E12-DS-3-E14 (2-(4-(2-((3-(Bis(2-hydroxytetradecyl)amino)propyl)disulfaneyl)ethyl)piperazin-1-yl)ethyl 4-(bis(2-hydroxydodecyl)amino)butanoate), which is a HEPES-based disulfide cationic lipid with a piperazine core, having the Formula V:

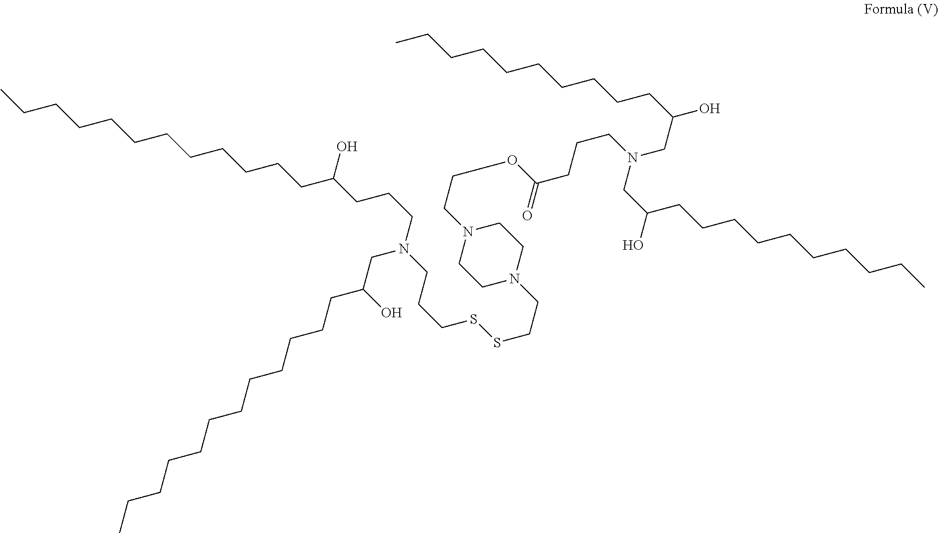 [SMILES: CCCCCCCCCCCCC(O)CCCN(CCCSSCCN1CCN(CCOC(=O)CCCN(CC(O)CCCCCCCCCC)CC(O)CCCCCCCCCC)CC1)CC(O)CCCCCCCCCCCC]

An exemplary LNP formulation herein, Lipid E, contains GL-HEPES-E3-E12-DS-3-E14. Lipid E has the same composition as Lipid A or Lipid B but for the difference in the cationic lipid.

The cationic lipids GL-HEPES-E3-E10-DS-3-E18-1 (III), GL-HEPES-E3-E12-DS-4-E10 (IV), and GL-HEPES-E3-E12-DS-3-E14 (V) can be synthesized according to the general procedure set out in Scheme 1:

### Scheme 1: General Synthetic Scheme for Lipids of Formulas (III), (IV), and (V)

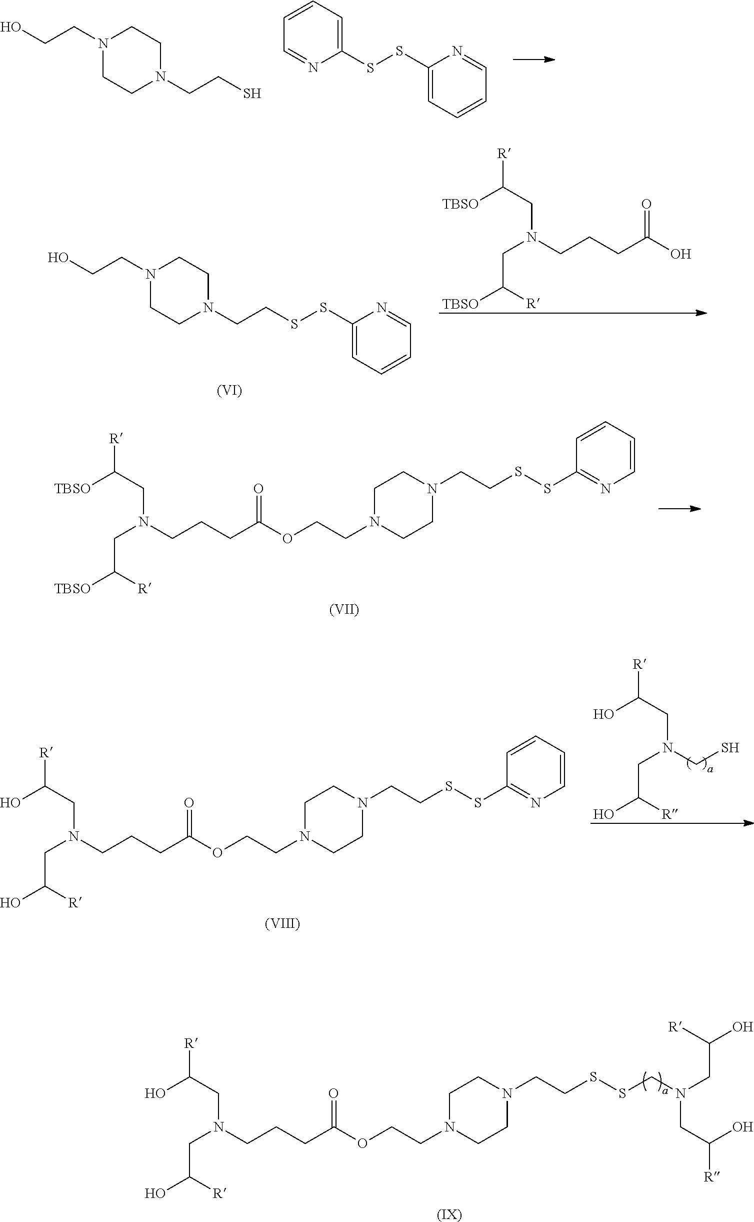

In some embodiments, the cationic lipid is MC3, having the Formula VI:

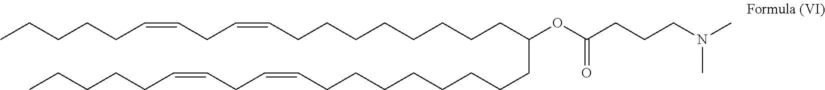 [SMILES: CCCCC/C=C\C/C=C\CCCCCCCCC(CCCCCCCC/C=C\C/C=C\CCCCC)OC(=O)CCCN(C)C]

In some embodiments, the cationic lipid is SM-102 (9-heptadecanyl 8-{(2-hydroxyethyl)[6-oxo-6-(undecyloxy)hexyl]amino}octanoate), having the Formula VII:

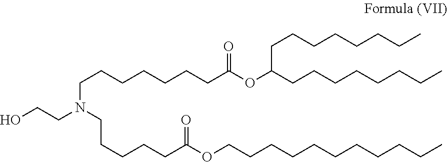 [SMILES: CCCCCCCCCCCOC(=O)CCCCCN(CCO)CCCCCCCC(=O)OC(CCCCCCCC)CCCCCCCC]

In some embodiments, the cationic lipid is ALC-0315 [(4-hydroxybutyl)azanediyl]di(hexane-6,1-diyl) bis(2-hexyldecanoate), having the Formula VIII:

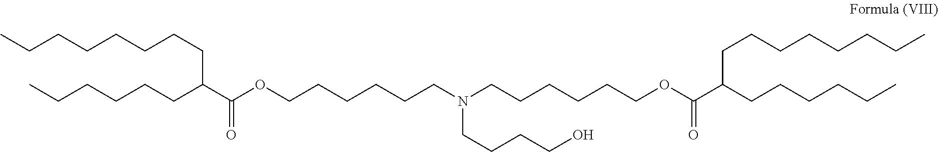 [SMILES: CCCCCCCCC(CCCCCC)C(=O)OCCCCCCN(CCCCO)CCCCCCOC(=O)C(CCCCCC)CCCCCCCC]

In some embodiments, the cationic lipid is cOrn-EE1, having the Formula IX:

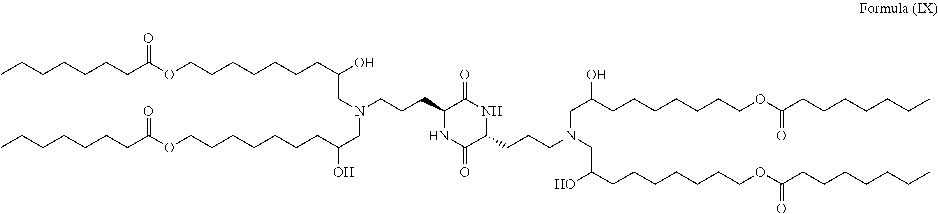 [SMILES: CCCCCCCC(=O)OCCCCCCCC(O)CN(CCC[C@@H]1NC(=O)[C@@H](CCCN(CC(O)CCCCCCCOC(=O)CCCCCCC)CC(O)CCCCCCCOC(=O)CCCCCCC)NC1=O)CC(O)CCCCCCCOC(=O)CCCCCCC]

In some embodiments, the cationic lipid may be selected from the group comprising cKK-E10; OF-02; [(6Z,9Z,28Z,31Z)-heptatriaconta-6,9,28,31-tetraen-19-yl] 4-(dimethylamino) butanoate (D-Lin-MC3-DMA); 2,2-dilinoleyl-4-dimethylaminoethyl-[1,3]-dioxolane (DLin-KC2-DMA); 1,2-dilinoleyloxy-N,N-dimethyl-3-aminopropane (DLin-DMA); di((Z)-non-2-en-1-yl) 9-((4-(dimethylamino) butanoyl)oxy) heptadecanedioate (L319); 9-heptadecanyl 8-{(2-hydroxyethyl)[6-oxo-6-(undecyloxy)hexyl]amino}octanoate (SM-102); [(4-hydroxybutyl)azanediyl]di(hexane-6,1-diyl) bis(2-hexyldecanoate) (ALC-0315); [3-(dimethylamino)-2-[(Z)-octadec-9-enoyl]oxypropyl] (Z)-octadec-9-enoate (DODAP); 2,5-bis(3-aminopropylamino)-N-[2-[di(heptadecyl)amino]-2-oxoethyl]pentanamide (DOGS); [(3S,8S,9S,10R,13R,14S,17R)-10,13-dimethyl-17-[(2R)-6-methylheptan-2-yl]-2,3,4,7,8,9,11,12,14,15,16,17-dodecahydro-1H-cyclopenta[a]phenanthren-3-yl] N-[2-(dimethylamino)ethyl]carbamate (DC-Chol); tetrakis(8-methylnonyl) 3,3′,3″,3″-(((methylazanediyl) bis(propane-3,1 diyl))bis (azanetriyl))tetrapropionate (306Oi10); decyl (2-(dioctylammonio)ethyl) phosphate (9A1P9); ethyl 5,5-di((Z)-heptadec-8-en-1-yl)-1-(3-(pyrrolidin-1-yl) propyl)-2,5-dihydro-1H-imidazole-2-carboxylate (A2-Iso5-2DC18); bis(2-(dodecyldisulfanyl)ethyl) 3,3′-((3-methyl-9-oxo-10-oxa-13,14-dithia-3,6-diazahexacosyl)azanediyl)dipropionate (BAME-O16B); 1,1′-((2-(4-(2-((2-(bis(2-hydroxydodecyl)amino)ethyl) (2-hydroxydodecyl)amino)ethyl) piperazin-1-yl)ethyl)azanediyl) bis(dodecan-2-ol) (C12-200); 3,6-bis(4-(bis(2-hydroxydodecyl)amino)butyl)piperazine-2,5-dione (cKK-E12); hexa(octan-3-yl) 9,9′,9″,9″,9″″,9″″-((((benzene-1,3,5-tricarbonyl)yris(azanediyl)) tris (propane-3,1-diyl)) tris(azanetriyl))hexanonanoate (FTT5); (((3,6-dioxopiperazine-2,5-diyl)bis(butane-4,1-diyl))bis(azanetriyl))tetrakis(ethane-2,1-diyl) (9Z,9′Z,9″Z,9″Z, 12Z, 12′Z, 12″Z, 12″Z)-tetrakis(octadeca-9,12-dienoate) (OF-Deg-Lin); TT3; N¹,N³,N⁵-tris(3-(didodecylamino) propyl)benzene-1,3,5-tricarboxamide; N1-[2-((1S)-1-[(3-aminopropyl)amino]-4-[di(3-aminopropyl)amino]butylcarboxamido)ethyl]-3,4-di[oleyloxy]-benzamide (MVL5); heptadecan-9-yl 8-((2-hydroxyethyl) (8-(nonyloxy)-8-oxooctyl)amino) octanoate (Lipid 5); GL-HEPES-E3-E10-DS-3-E18-1; GL-HEPES-E3-E12-DS-4-E10; GL-HEPES-E3-E12-DS-3-E14; and combinations thereof.

In some embodiments, the cationic lipid is biodegradable.

In some embodiments, the cationic lipid is not biodegradable.

In some embodiments, the cationic lipid is cleavable.

In some embodiments, the cationic lipid is not cleavable.

Cationic lipids are described in further detail in Dong et al. (PNAS. 111 (11): 3955-60. 2014); Fenton et al. (Adv Mater. 28:2939. 2016); U.S. Pat. Nos. 9,512,073; and 10,201,618, each of which is incorporated herein by reference.

### B. PEGylated Lipid

The PEGylated lipid component provides control over particle size and stability of the nanoparticle. The addition of such components may prevent complex aggregation and provide a means for increasing circulation lifetime and increasing the delivery of the lipid-nucleic acid pharmaceutical composition to target tissues (Klibanov et al. FEBS Letters 268 (1): 235-7. 1990). These components may be selected to rapidly exchange out of the pharmaceutical composition in vivo (see, e.g., U.S. Pat. No. 5,885,613).

Contemplated PEGylated lipids include, but are not limited to, a polyethylene glycol (PEG) chain of up to 5 kDa in length covalently attached to a lipid with alkyl chain(s) of C₆-C₂₀ (e.g., C₈, C₁₀, C₁₂, C₁₄, C₁₆, or C₁₈) length, such as a derivatized ceramide (e.g., N-octanoyl-sphingosine-1-[succinyl(methoxypolyethylene glycol)] (C8 PEG ceramide)). In some embodiments, the PEGylated lipid is 1,2-dimyristoyl-rac-glycero-3-methoxypolyethylene glycol (DMG-PEG); 1,2-distearoyl-sn-glycero-3-phosphoethanolamine-polyethylene glycol (DSPE-PEG); 1,2-dilauroyl-sn-glycero-3-phosphoethanolamine-polyethylene glycol (DLPE-PEG); 1,2-distearoyl-rac-glycero-polyethelene glycol (DSG-PEG), PEG-DAG; PEG-PE; PEG-S-DAG; PEG-S-DMG; PEG-cer; a PEG-dialkyoxypropylcarbamate; 2-[(polyethylene glycol)-2000]-N,N-ditetradecylacetamide (ALC-0159); and combinations thereof.

In certain embodiments, the PEG has a high molecular weight, e.g., 2000-2400 g/mol. In certain embodiments, the PEG is PEG2000 (or PEG-2K). In certain embodiments, the PEGylated lipid herein is DMG-PEG2000, DSPE-PEG2000, DLPE-PEG2000, DSG-PEG2000, C8 PEG2000, or ALC-0159 (2-[(polyethylene glycol)-2000]-N,N-ditetradecylacetamide). In certain embodiments, the PEGylated lipid herein is DMG-PEG2000.

### C. Cholesterol-Based Lipid

The cholesterol component provides stability to the lipid bilayer structure within the nanoparticle. In some embodiments, the LNPs comprise one or more cholesterol-based lipids. Suitable cholesterol-based lipids include, for example: DC-Choi (N,N-dimethyl-N-ethylcarboxamidocholesterol), 1,4-bis(3-N-oleylamino-propyl) piperazine (Gao et al., Biochem Biophys Res Comm. (1991) 179:280; Wolf et al., BioTechniques (1997) 23:139; U.S. Pat. No. 5,744,335), imidazole cholesterol ester (“ICE”; WO 2011/068810), sitosterol (22,23-dihydrostigmasterol), β-sitosterol, sitostanol, fucosterol, stigmasterol (stigmasta-5,22-dien-3-ol), ergosterol; desmosterol (3β-hydroxy-5,24-cholestadiene); lanosterol (8,24-lanostadien-3b-ol); 7-dehydrocholesterol (Δ5,7-cholesterol); dihydrolanosterol (24,25-dihydrolanosterol); zymosterol (5α-cholesta-8,24-dien-3β-ol); lathosterol (5α-cholest-7-en-3β-ol); diosgenin ((3β,25R)-spirost-5-en-3-ol); campesterol (campest-5-en-3β-ol); campestanol (5a-campestan-3b-ol); 24-methylene cholesterol (5,24 (28)-cholestadien-24-methylen-3β-ol); cholesteryl margarate (cholest-5-en-3β-yl heptadecanoate); cholesteryl oleate; cholesteryl stearate and other modified forms of cholesterol. In some embodiments, the cholesterol-based lipid used in the LNPs is cholesterol.

### D. Helper Lipid

A helper lipid enhances the structural stability of the LNP and helps the LNP in endosome escape. It improves uptake and release of the mRNA drug payload. In some embodiments, the helper lipid is a zwitterionic lipid, which has fusogenic properties for enhancing uptake and release of the drug payload. Examples of helper lipids are 1,2-dioleoyl-SN-glycero-3-phosphoethanolamine (DOPE); 1,2-distearoyl-sn-glycero-3-phosphocholine (DSPC); 1,2-dioleoyl-sn-glycero-3-phospho-L-serine (DOPS); 1,2-dielaidoyl-sn-glycero-3-phosphoethanolamine (DEPE); and 1,2-dioleoyl-sn-glycero-3-phosphocholine (DPOC), dipalmitoylphosphatidylcholine (DPPC), DMPC, 1,2-dilauroyl-sn-glycero-3-phosphocholine (DLPC), 1,2-Distearoylphosphatidylethanolamine (DSPE), and 1,2-dilauroyl-sn-glycero-3-phosphoethanolamine (DLPE).

Other exemplary helper lipids are dioleoylphosphatidylcholine (DOPC), dioleoylphosphatidylglycerol (DOPG), dipalmitoylphosphatidylglycerol (DPPG), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-I-carboxylate (DOPE-mal), dipalmitoyl phosphatidyl ethanolamine (DPPE), dimyristoylphosphoethanolamine (DMPE), phosphatidylserine, sphingolipids, sphingomyelins, ceramides, cerebrosides, gangliosides, 16-O-monomethyl PE, 16-O-dimethyl PE, 18-1-trans PE, I-stearoyl-2-oleoyl-phosphatidyethanolamine (SOPE), or a combination thereof. In certain embodiments, the helper lipid is DOPE. In certain embodiments, the helper lipid is DSPC.

In other embodiments, the present LNPs comprise (i) SM-102; (ii) DMG-PEG2000; (iii) cholesterol; and (iv) DSPC.

In yet other embodiments, the present LNPs comprise (i) ALC-0315; (ii) ALC-0159; (iii) cholesterol; and (iv) DSPC.

In yet other embodiments, the present LNPs comprise (i) OF-02; (ii) DMG-PEG2000; cholesterol; and (iv) DOPE.

In yet other embodiments, the present LNPs comprise (i) cKK-E10; (ii) DMG-PEG2000; (iii) cholesterol; and (iv) DOPE.

In yet other embodiments, the present LNPs comprise (i) GL-HEPES-E3-E10-DS-3-E18-1; (ii) DMG-PEG2000; (iii) cholesterol; and (iv) DOPE.

In yet other embodiments, the present LNPs comprise (i) GL-HEPES-E3-E12-DS-4-E10; (ii) DMG-PEG2000; (iii) cholesterol; and (iv) DOPE.

In yet other embodiments, the present LNPs comprise (i) GL-HEPES-E3-E12-DS-3-E14; (ii) DMG-PEG2000; (iii) cholesterol; and (iv) DOPE.

### E. Molar Ratios of the Lipid Components

The molar ratios of the above components are important for the LNPs' effectiveness in delivering mRNA. The molar ratio of the cationic lipid, the PEGylated lipid, the cholesterol-based lipid, and the helper lipid is A:B:C:D, where A+B+C+D=100%. In some embodiments, the molar ratio of the cationic lipid in the LNPs relative to the total lipids (i.e., A) is 35-55%, such as 35-50% (e.g., 38-42% such as 40%, or 45-50%). In some embodiments, the molar ratio of the PEGylated lipid component relative to the total lipids (i.e., B) is 0.25-2.75% (e.g., 1-2% such as 1.5%). In some embodiments, the molar ratio of the cholesterol-based lipid relative to the total lipids (i.e., C) is 20-50% (e.g., 27-30% such as 28.5%, or 38-43%). In some embodiments, the molar ratio of the helper lipid relative to the total lipids (i.e., D) is 5-35% (e.g., 28-32% such as 30%, or 8-12%, such as 10%). In some embodiments, the (PEGylated lipid+cholesterol) components have the same molar amount as the helper lipid. In some embodiments, the LNPs contain a molar ratio of the cationic lipid to the helper lipid that is more than 1.

In certain embodiments, the LNP of the disclosure comprises:
a cationic lipid at a molar ratio of 35% to 55% or 40% to 50% (e.g., a cationic lipid at a molar ratio of 35%, 36%, 37%, 38%, 39%, 40%, 41% 42%, 43%, 44%, 45%, 46%, 47%, 48%, 49%, 50%, 51%, 52%, 53%, 54%, or 55%);
a polyethylene glycol (PEG) conjugated (PEGylated) lipid at a molar ratio between 0.25% and 2.75% or between 1.00% and 2.00% (e.g., a PEGylated lipid at a molar ratio of 0.25%, 0.50%, 0.75%, 1.00%, 1.25%, 1.50%, 1.75%, 2.00%, 2.25%, 2.50%, or 2.75%);
a cholesterol-based lipid at a molar ratio of 20% to 50%, 25% to 45%, or 28.5% to 43% (e.g., a cholesterol-based lipid at a molar ratio of 20%, 21%, 22%, 23%, 24%, 25%, 26%, 27%, 28%, 29%, 30%, 31%, 32%, 33%, 34%, 35%, 36%, 37%, 38%, 39%, 40%, 41% 42%, 43%, 44%, 45%, 46%, 47%, 48%, 49%, or 50%); and
a helper lipid at a molar ratio of 5% to 35%, 8% to 30%, or 10% to 30% (e.g., a helper lipid at a molar ratio of 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, or 35%),
wherein all of the molar ratios are relative to the total lipid content of the LNP.

In certain embodiments, the LNP comprises: a cationic lipid at a molar ratio of 40%; a PEGylated lipid at a molar ratio of 1.5%; a cholesterol-based lipid at a molar ratio of 28.5%; and a helper lipid at a molar ratio of 30%.

In certain embodiments, the LNP of the disclosure comprises: a cationic lipid at a molar ratio of 45 to 50%; a PEGylated lipid at a molar ratio of 1.5 to 1.7%; a cholesterol-based lipid at a molar ratio of 38 to 43%; and a helper lipid at a molar ratio of 9 to 10%.

In certain embodiments, the PEGylated lipid is dimyristoyl-PEG2000 (DMG-PEG2000).

In certain embodiments, the cholesterol-based lipid is cholesterol.

In certain embodiments, the helper lipid is 1,2-dioleoyl-SN-glycero-3-phosphoethanolamine (DOPE).

In certain embodiments, the LNP comprises: OF-02 at a molar ratio of 35% to 55%; DMG-PEG2000 at a molar ratio of 0.25% to 2.75%; cholesterol at a molar ratio of 20% to 50%; and DOPE at a molar ratio of 5% to 35%.

In certain embodiments, the LNP comprises: cKK-E10 at a molar ratio of 35% to 55%; DMG-PEG2000 at a molar ratio of 0.25% to 2.75%; cholesterol at a molar ratio of 20% to 50%; and DOPE at a molar ratio of 5% to 35%.

In certain embodiments, the LNP comprises: GL-HEPES-E3-E10-DS-3-E18-1 at a molar ratio of 35% to 55%; DMG-PEG2000 at a molar ratio of 0.25% to 2.75%; cholesterol at a molar ratio of 20% to 50%; and DOPE at a molar ratio of 5% to 35%.

In certain embodiments, the LNP comprises: GL-HEPES-E3-E12-DS-4-E10 at a molar ratio of 35% to 55%; DMG-PEG2000 at a molar ratio of 0.25% to 2.75%; cholesterol at a molar ratio of 20% to 50%; and DOPE at a molar ratio of 5% to 35%.

In certain embodiments, the LNP comprises: GL-HEPES-E3-E12-DS-3-E14 at a molar ratio of 35% to 55%; DMG-PEG2000 at a molar ratio of 0.25% to 2.75%; cholesterol at a molar ratio of 20% to 50%; and DOPE at a molar ratio of 5% to 35%.

In certain embodiments, the LNP comprises: SM-102 at a molar ratio of 35% to 55%; DMG-PEG2000 at a molar ratio of 0.25% to 2.75%; cholesterol at a molar ratio of 20% to 50%; and DSPC at a molar ratio of 5% to 35%.

In certain embodiments, the LNP comprises: ALC-0315 at a molar ratio of 35% to 55%; ALC-0159 at a molar ratio of 0.25% to 2.75%; cholesterol at a molar ratio of 20% to 50%; and DSPC at a molar ratio of 5% to 35%.

In certain embodiments, the LNP comprises: OF-02 at a molar ratio of 40%; DMG-PEG2000 at a molar ratio of 1.5%; cholesterol at a molar ratio of 28.5%; and DOPE at a molar ratio of 30%. This LNP formulation is designated “Lipid A” herein.

In certain embodiments, the LNP comprises: cKK-E10 at a molar ratio of 40%; DMG-PEG2000 at a molar ratio of 1.5%; cholesterol at a molar ratio of 28.5%; and DOPE at a molar ratio of 30%. This LNP formulation is designated “Lipid B” herein.

In certain embodiments, the LNP comprises: GL-HEPES-E3-E10-DS-3-E18-1 at a molar ratio of 40%; DMG-PEG2000 at a molar ratio of 1.5%; cholesterol at a molar ratio of 28.5%; and DOPE at a molar ratio of 30%. This LNP formulation is designated “Lipid C” herein.

In certain embodiments, the LNP comprises: GL-HEPES-E3-E12-DS-4-E10 (at a molar ratio of 40%; DMG-PEG2000 at a molar ratio of 1.5%; cholesterol at a molar ratio of 28.5%; and DOPE at a molar ratio of 30%. This LNP formulation is designated “Lipid D” herein.

In certain embodiments, the LNP comprises: GL-HEPES-E3-E12-DS-3-E14 at a molar ratio of 40%; DMG-PEG2000 at a molar ratio of 1.5%; cholesterol at a molar ratio of 28.5%; and DOPE at a molar ratio of 30%. This LNP formulation is designated “Lipid E” herein.

In certain embodiments, the LNP comprises: 9-heptadecanyl 8-{(2-hydroxyethyl) [6-oxo-6-(undecyloxy)hexyl]amino}octanoate (SM-102) at a molar ratio of 50%; 1,2-distearoyl-sn-glycero-3-phosphocholine (DSPC) at a molar ratio of 10%; cholesterol at a molar ratio of 38.5%; and 1,2-dimyristoyl-rac-glycero-3-methoxypolyethylene glycol-2000 (DMG-PEG2000) at a molar ratio of 1.5%.

In certain embodiments, the LNP comprises: (4-hydroxybutyl) azanediyl]di(hexane-6,1-diyl) bis(2-hexyldecanoate) (ALC-0315) at a molar ratio of 46.3%; 1,2-distearoyl-sn-glycero-3-phosphocholine (DSPC) at a molar ratio of 9.4%; cholesterol at a molar ratio of 42.7%; and 2-[(polyethylene glycol)-2000]-N,N-ditetradecylacetamide (ALC-0159) at a molar ratio of 1.6%.

In certain embodiments, the LNP comprises: (4-hydroxybutyl)azanediyl]di(hexane-6,1-diyl) bis(2-hexyldecanoate) (ALC-0315) at a molar ratio of 47.4%; 1,2-distearoyl-sn-glycero-3-phosphocholine (DSPC) at a molar ratio of 10%; cholesterol at a molar ratio of 40.9%; and 2-[(polyethylene glycol)-2000]-N,N-ditetradecylacetamide (ALC-0159) at a molar ratio of 1.7%.

To calculate the actual amount of each lipid to be put into an LNP formulation, the molar amount of the cationic lipid is first determined based on a desired N/P ratio, where N is the number of nitrogen atoms in the cationic lipid and P is the number of phosphate groups in the mRNA to be transported by the LNP. Next, the molar amount of each of the other lipids is calculated based on the molar amount of the cationic lipid and the molar ratio selected. These molar amounts are then converted to weights using the molecular weight of each lipid.

### F. Active Ingredients of the LNPs

The active ingredient of the present LNP vaccine composition is a nucleic acid (e.g., a mRNA) that encodes an antigenic polypeptide derived from at least one bacteria of the genus Borrelia.

Where desired, the LNP may be multi-valent. In some embodiments, the LNP may carry nucleic acids, such as mRNAs, that encode more than one antigenic polypeptide derived from at least one bacteria of the genus Borrelia, such as two, three, four, five, six, seven, or eight antigens. For example, the LNP may carry multiple nucleic acids (e.g., mRNA), each encoding a different antigenic polypeptide derived from at least one bacteria of the genus Borrelia; or carry a polycistronic mRNA that can be translated into more than one antigenic polypeptide derived from at least one bacteria of the genus Borrelia (e.g., each antigen-coding sequence is separated by a nucleotide linker encoding a self-cleaving peptide such as a 2A peptide). An LNP carrying different nucleic acids (e.g., mRNA) typically comprises (encapsulate) multiple copies of each nucleic acid. For example, an LNP carrying or encapsulating two different nucleic acids typically carries multiple copies of each of the two different nucleic acids.

In some embodiments, a single LNP formulation may comprise multiple kinds (e.g., two, three, four, five, six, seven, eight, nine, ten, or more) of LNPs, each kind carrying a different nucleic acid (e.g., mRNA).

When the nucleic acid is mRNA, the mRNA may be unmodified (i.e., containing only natural ribonucleotides A, U, C, and/or G linked by phosphodiester bonds), or chemically modified (e.g., including nucleotide analogs such as pseudouridines (e.g., N-1-methyl pseudouridine), 2′-fluoro ribonucleotides, and 2′-methoxy ribonucleotides, and/or phosphorothioate bonds). The mRNA molecule may comprise a 5′ cap and a polyA tail.

### G. Buffer and Other Components

To stabilize the nucleic acid and/or LNPs (e.g., to prolong the shelf-life of the vaccine product), to facilitate administration of the LNP pharmaceutical composition, and/or to enhance in vivo expression of the nucleic acid, the nucleic acid and/or LNP can be formulated in combination with one or more carriers, targeting ligands, stabilizing reagents (e.g., preservatives and antioxidants), and/or other pharmaceutically acceptable excipients. Examples of such excipients are parabens, thimerosal, thiomersal, chlorobutanol, bezalkonium chloride, and chelators (e.g., EDTA).

The LNP compositions of the present disclosure can be provided as a frozen liquid form or a lyophilized form. A variety of cryoprotectants may be used, including, without limitations, sucrose, trehalose, glucose, mannitol, mannose, dextrose, and the like. The cryoprotectant may constitute 5-30% (w/v) of the LNP composition. In some embodiments, the LNP composition comprise trehalose, e.g., at 5-30% (e.g., 10%) (w/v). Once formulated with the cryoprotectant, the LNP compositions may be frozen (or lyophilized and cryopreserved) at −20° C. to −80° C.

The LNP compositions may be provided to a patient in an aqueous buffered solution-thawed if previously frozen, or if previously lyophilized, reconstituted in an aqueous buffered solution at bedside. The buffered solution typically is isotonic and suitable for e.g., intramuscular or intradermal injection. In some embodiments, the buffered solution is a phosphate-buffered saline (PBS).

### V. Processes for Making LNP Vaccines

The present LNPs can be prepared by various techniques presently known in the art. For example, multilamellar vesicles (MLV) may be prepared according to conventional techniques, such as by depositing a selected lipid on the inside wall of a suitable container or vessel by dissolving the lipid in an appropriate solvent, and then evaporating the solvent to leave a thin film on the inside of the vessel or by spray drying. An aqueous phase may then be added to the vessel with a vortexing motion that results in the formation of MLVs. Unilamellar vesicles (ULV) can then be formed by homogenization, sonication or extrusion of the multilamellar vesicles. In addition, unilamellar vesicles can be formed by detergent removal techniques.

Various methods are described in US 2011/0244026, US 2016/0038432, US 2018/0153822, US 2018/0125989, and US 2021/0046192 and can be used to practice the present disclosure. One exemplary process entails encapsulating mRNA by mixing it with a mixture of lipids, without first pre-forming the lipids into lipid nanoparticles, as described in US 2016/0038432. Another exemplary process entails encapsulating mRNA by mixing pre-formed LNPs with mRNA, as described in US 2018/0153822.

In some embodiments, the process of preparing mRNA-loaded LNPs includes a step of heating one or more of the solutions to a temperature greater than ambient temperature, the one or more solutions being the solution comprising the pre-formed lipid nanoparticles, the solution comprising the mRNA and the mixed solution comprising the LNP-encapsulated mRNA. In some embodiments, the process includes the step of heating one or both of the mRNA solution and the pre-formed LNP solution, prior to the mixing step. In some embodiments, the process includes heating one or more of the solutions comprising the pre-formed LNPs, the solution comprising the mRNA and the solution comprising the LNP-encapsulated mRNA, during the mixing step. In some embodiments, the process includes the step of heating the LNP-encapsulated mRNA, after the mixing step. In some embodiments, the temperature to which one or more of the solutions is heated is or is greater than about 30° C., 37° C., 40° C., 45° C., 50° C., 55° C., 60° C., 65° C., or 70° C. In some embodiments, the temperature to which one or more of the solutions is heated ranges from about 25-70° C., about 30-70° C., about 35-70° C., about 40-70° C., about 45-70° C., about 50-70° C., or about 60-70° C. In some embodiments, the temperature is about 65° C.

Various methods may be used to prepare an mRNA solution suitable for the present disclosure. In some embodiments, mRNA may be directly dissolved in a buffer solution described herein. In some embodiments, an mRNA solution may be generated by mixing an mRNA stock solution with a buffer solution prior to mixing with a lipid solution for encapsulation. In some embodiments, an mRNA solution may be generated by mixing an mRNA stock solution with a buffer solution immediately before mixing with a lipid solution for encapsulation. In some embodiments, a suitable mRNA stock solution may contain mRNA in water or a buffer at a concentration at or greater than about 0.2 mg/ml, 0.4 mg/ml, 0.5 mg/ml, 0.6 mg/ml, 0.8 mg/ml, 1.0 mg/ml, 1.2 mg/ml, 1.4 mg/ml, 1.5 mg/ml, or 1.6 mg/ml, 2.0 mg/ml, 2.5 mg/ml, 3.0 mg/ml, 3.5 mg/ml, 4.0 mg/ml, 4.5 mg/ml, or 5.0 mg/ml.

In some embodiments, an mRNA stock solution is mixed with a buffer solution using a pump. Exemplary pumps include but are not limited to gear pumps, peristaltic pumps and centrifugal pumps. Typically, the buffer solution is mixed at a rate greater than that of the mRNA stock solution. For example, the buffer solution may be mixed at a rate at least 1×, 2×, 3×, 4×, 5×, 6×, 7×, 8×, 9×, 10×, 15×, or 20× greater than the rate of the mRNA stock solution. In some embodiments, a buffer solution is mixed at a flow rate ranging between about 100-6000 ml/minute (e.g., about 100-300 ml/minute, 300-600 ml/minute, 600-1200 ml/minute, 1200-2400 ml/minute, 2400-3600 ml/minute, 3600-4800 ml/minute, 4800-6000 ml/minute, or 60-420 ml/minute). In some embodiments, a buffer solution is mixed at a flow rate of, or greater than, about 60 ml/minute, 100 ml/minute, 140 ml/minute, 180 ml/minute, 220 ml/minute, 260 ml/minute, 300 ml/minute, 340 ml/minute, 380 ml/minute, 420 ml/minute, 480 ml/minute, 540 ml/minute, 600 ml/minute, 1200 ml/minute, 2400 ml/minute, 3600 ml/minute, 4800 ml/minute, or 6000 ml/minute.

In some embodiments, an mRNA stock solution is mixed at a flow rate ranging between about 10-600 ml/minute (e.g., about 5-50 ml/minute, about 10-30 ml/minute, about 30-60 ml/minute, about 60-120 ml/minute, about 120-240 ml/minute, about 240-360 ml/minute, about 360-480 ml/minute, or about 480-600 ml/minute). In some embodiments, an mRNA stock solution is mixed at a flow rate of or greater than about 5 ml/minute, 10 ml/minute, 15 ml/minute, 20 ml/minute, 25 ml/minute, 30 ml/minute, 35 ml/minute, 40 ml/minute, 45 ml/minute, 50 ml/minute, 60 ml/minute, 80 ml/minute, 100 ml/minute, 200 ml/minute, 300 ml/minute, 400 ml/minute, 500 ml/minute, or 600 ml/minute.

The process of incorporation of a desired mRNA into a lipid nanoparticle is referred to as “loading.” Exemplary methods are described in Lasic et al., FEBS Lett. (1992) 312:255-8. The LNP-incorporated nucleic acids may be completely or partially located in the interior space of the lipid nanoparticle, within the bilayer membrane of the lipid nanoparticle, or associated with the exterior surface of the lipid nanoparticle membrane. The incorporation of an mRNA into lipid nanoparticles is also referred to herein as “encapsulation” wherein the nucleic acid is entirely or substantially contained within the interior space of the lipid nanoparticle.

Suitable LNPs may be made in various sizes. In some embodiments, decreased size of lipid nanoparticles is associated with more efficient delivery of an mRNA. Selection of an appropriate LNP size may take into consideration the site of the target cell or tissue and to some extent the application for which the lipid nanoparticle is being made.

A variety of methods known in the art are available for sizing of a population of lipid nanoparticles. Exemplary methods herein utilize Zetasizer Nano ZS (Malvern Panalytical) to measure LNP particle size. In one protocol, 10 μl of an LNP sample are mixed with 990 μl of 10% trehalose. This solution is loaded into a cuvette and then put into the Zetasizer machine. The z-average diameter (nm), or cumulants mean, is regarded as the average size for the LNPs in the sample. The Zetasizer machine can also be used to measure the polydispersity index (PDI) by using dynamic light scattering (DLS) and cumulant analysis of the autocorrelation function. Average LNP diameter may be reduced by sonication of formed LNP. Intermittent sonication cycles may be alternated with quasi-elastic light scattering (QELS) assessment to guide efficient lipid nanoparticle synthesis.

In some embodiments, the majority of purified LNPs, i.e., greater than about 50%, 55%, 60%, 65%, 70%, 75%, 80%, 85%, 90%, 95%, 96%, 97%, 98%, or 99% of the LNPs, have a size of about 70-150 nm (e.g., about 145 nm, about 140 nm, about 135 nm, about 130 nm, about 125 nm, about 120 nm, about 115 nm, about 110 nm, about 105 nm, about 100 nm, about 95 nm, about 90 nm, about 85 nm, or about 80 nm). In some embodiments, substantially all (e.g., greater than 80 or 90%) of the purified lipid nanoparticles have a size of about 70-150 nm (e.g., about 145 nm, about 140 nm, about 135 nm, about 130 nm, about 125 nm, about 120 nm, about 115 nm, about 110 nm, about 105 nm, about 100 nm, about 95 nm, about 90 nm, about 85 nm, or about 80 nm).

In certain embodiments, the LNP has an average diameter of 30-200 nm.

In certain embodiments, the LNP has an average diameter of 80-150 nm.

In some embodiments, the LNPs in the present composition have an average size of less than 150 nm, less than 120 nm, less than 100 nm, less than 90 nm, less than 80 nm, less than 70 nm, less than 60 nm, less than 50 nm, less than 30 nm, or less than 20 nm.

In some embodiments, greater than about 70%, 75%, 80%, 85%, 90%, 95%, 96%, 97%, 98%, 99% of the LNPs in the present composition have a size ranging from about 40-90 nm (e.g., about 45-85 nm, about 50-80 nm, about 55-75 nm, about 60-70 nm), about 40-90 nm (e.g., about 45-85 nm, about 50-80 nm, about 55-75 nm, about 60-70 nm), or about 50-70 nm (e.g., 55-65 nm) are particular suitable for pulmonary delivery via nebulization.

In some embodiments, the dispersity, or measure of heterogeneity in size of molecules (PDI), of LNPs in a pharmaceutical composition provided by the present disclosure is less than about 0.5. In some embodiments, an LNP has a PDI of less than about 0.5, less than about 0.4, less than about 0.3, less than about 0.28, less than about 0.25, less than about 0.23, less than about 0.20, less than about 0.18, less than about 0.16, less than about 0.14, less than about 0.12, less than about 0.10, or less than about 0.08. The PDI may be measured by a Zetasizer machine as described above.

In some embodiments, greater than about 75%, 80%, 85%, 90%, 95%, 96%, 97%, 98%, or 99% of the purified LNPs in a pharmaceutical composition provided herein encapsulate an mRNA within each individual particle. In some embodiments, substantially all (e.g., greater than 80% or 90%) of the purified lipid nanoparticles in a pharmaceutical composition encapsulate an mRNA within each individual particle. In some embodiments, a lipid nanoparticle has an encapsulation efficiency of between 50% and 99%; or greater than about 60, 65, 70, 75, 80, 85, 90, 92, 95, 98, or 99%. Typically, lipid nanoparticles for use herein have an encapsulation efficiency of at least 90% (e.g., at least 91, 92, 93, 94, or 95%).

In some embodiments, an LNP has a N/P ratio of between 1 and 10. In some embodiments, a lipid nanoparticle has a N/P ratio above 1, about 1, about 2, about 3, about 4, about 5, about 6, about 7, or about 8. In further embodiments, a typical LNP herein has an N/P ratio of 4.

In some embodiments, a pharmaceutical composition according to the present disclosure contains at least about 0.5 μg, 1 μg, 5 μg, 10 μg, 100 μg, 500 μg, or 1000 μg of encapsulated mRNA. In some embodiments, a pharmaceutical composition contains about 0.1 μg to 1000 μg, at least about 0.5 μg, at least about 0.8 μg, at least about 1 μg, at least about 5 μg, at least about 8 μg, at least about 10 μg, at least about 50 μg, at least about 100 μg, at least about 500 μg, or at least about 1000 μg of encapsulated mRNA.

In some embodiments, mRNA can be made by chemical synthesis or by in vitro transcription (IVT) of a DNA template. An exemplary process for making and purifying mRNA is described in Example 1. In this process, in an IVT process, a cDNA template is used to produce an mRNA transcript and the DNA template is degraded by a DNase. The transcript is purified by depth filtration and tangential flow filtration (TFF). The purified transcript is further modified by adding a cap and a tail, and the modified RNA is purified again by depth filtration and TFF.

The mRNA is then prepared in an aqueous buffer and mixed with an amphiphilic solution containing the lipid components of the LNPs. An amphiphilic solution for dissolving the four lipid components of the LNPs may be an alcohol solution. In some embodiments, the alcohol is ethanol. The aqueous buffer may be, for example, a citrate, phosphate, acetate, or succinate buffer and may have a pH of about 3.0-7.0, e.g., about 3.5, about 4.0, about 4.5, about 5.0, about 5.5, about 6.0, or about 6.5. The buffer may contain other components such as a salt (e.g., sodium, potassium, and/or calcium salts). In particular embodiments, the aqueous buffer has 1 mM citrate, 150 mM NaCl, pH 4.5.

An exemplary, nonlimiting process for making an mRNA-LNP composition involves mixing of a buffered mRNA solution with a solution of lipids in ethanol in a controlled homogeneous manner, where the ratio of lipids:mRNA is maintained throughout the mixing process. In this illustrative example, the mRNA is presented in an aqueous buffer containing citric acid monohydrate, tri-sodium citrate dihydrate, and sodium chloride. The mRNA solution is added to the solution (1 mM citrate buffer, 150 mM NaCl, pH 4.5). The lipid mixture of four lipids (e.g., a cationic lipid, a PEGylated lipid, a cholesterol-based lipid, and a helper lipid) is dissolved in ethanol. The aqueous mRNA solution and the ethanol lipid solution are mixed at a volume ratio of 4:1 in a “T” mixer with a near “pulseless” pump system. The resultant mixture is then subjected for downstream purification and buffer exchange. The buffer exchange may be achieved using dialysis cassettes or a TFF system. TFF may be used to concentrate and buffer-exchange the resulting nascent LNP immediately after formation via the T-mix process. The diafiltration process is a continuous operation, keeping the volume constant by adding appropriate buffer at the same rate as the permeate flow.

VI. Packaging and Use of the mRNA-LNP OspA Vaccine

The mRNA-LNP vaccines can be formulated or packaged for parenteral (e.g., intramuscular, intradermal or subcutaneous) administration or nasopharyngeal (e.g., intranasal) administration. The vaccine compositions may be in the form of an extemporaneous formulation, where the LNP composition is lyophilized and reconstituted with a physiological buffer (e.g., PBS) just before use. The vaccine compositions also may be shipped and provided in the form of an aqueous solution or a frozen aqueous solution and can be directly administered to subjects without reconstitution (after thawing, if previously frozen.

Accordingly, the present disclosure provides an article of manufacture, such as a kit, that provides the mRNA-LNP vaccine in a single container, or provides the mRNA-LNP vaccine in one container and a physiological buffer for reconstitution in another container. The container(s) may contain a single-use dosage or multi-use dosage. The containers may be pre-treated glass vials or ampules. The article of manufacture may include instructions for use as well.

In particular embodiments, the mRNA-LNP vaccine is provided for use in intramuscular (IM) injection. The vaccine can be injected to a subject at, e.g., his/her deltoid muscle in the upper arm. In some embodiments, the vaccine is provided in a pre-filled syringe or injector (e.g., single-chambered or multi-chambered). In some embodiments, the vaccine is provided for use in inhalation and is provided in a pre-filled pump, aerosolizer, or inhaler.

The mRNA-LNP vaccines can be administered to subjects in need thereof in a prophylactically effective amount, i.e., an amount that provides sufficient immune protection against a target pathogen for a sufficient amount of time (e.g., one year, two years, five years, ten years, or life-time). Sufficient immune protection may be, for example, prevention or alleviation of symptoms associated with infections by the pathogen. In some embodiments, multiple doses (e.g., two doses) of the vaccine are administered (e.g., injected) to subjects in need thereof to achieve the desired prophylactic effects. The doses (e.g., prime and booster doses) may be separated by an interval of e.g., 2 weeks, 3 weeks, 4 weeks, one month, two months, three months, four months, five months, six months, nine months, one year, two years, three years, five years, or ten years.

In some embodiments, a single dose of the mRNA-LNP vaccine contains 1-50 μg of mRNA (e.g., monovalent or multivalent). For example, a single dose may contain about 2.5 μg, about 5 μg, about 7.5 μg, about 10 μg, about 12.5 μg, or about 15 μg of the mRNA for intramuscular (IM) injection. In further embodiments, a multi-valent single dose of an LNP vaccine contains multiple (e.g., 2, 3, or 4) kinds of LNPs, each for a different antigen, and each kind of LNP has an mRNA amount of, e.g., 2.5 μg, about 5 μg, about 7.5 μg, about 10 μg, about 12.5 μg, or about 15 μg.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of, cell and tissue culture, molecular biology, virology, immunology, microbiology, genetics, analytical chemistry, synthetic organic chemistry, medicinal and pharmaceutical chemistry, and protein and nucleic acid chemistry and hybridization described herein are those well-known and commonly used in the art. Enzymatic reactions and purification techniques are performed according to manufacturer's specifications, as commonly accomplished in the art or as described herein. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety. Although a number of documents are cited herein, this citation does not constitute an admission that any of these documents forms part of the common general knowledge in the art. As used herein, the term “approximately” or “about” as applied to one or more values of interest refers to a value that is similar to a stated reference value. In certain embodiments, the term refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context.

### VII. Vectors

In one aspect, disclosed herein are vectors comprising the mRNA compositions disclosed herein. The RNA sequences encoding a protein of interest (e.g., mRNA encoding an OspA protein) can be cloned into a number of types of vectors. For example, the nucleic acids can be cloned into a vector including, but not limited to a plasmid, a phagemid, a phage derivative, an animal virus, and a cosmid. Vectors of particular interest include expression vectors, replication vectors, probe generation vectors, sequencing vectors and vectors optimized for in vitro transcription.

In certain embodiments, the vector can be used to express mRNA in a host cell. In various embodiments, the vector is used as a template for IVT. The construction of optimally translated IVT mRNA suitable for therapeutic use is disclosed in detail in Sahin, et al. (2014). Nat. Rev. Drug Discov. 13, 759-780; Weissman (2015). Expert Rev. Vaccines 14, 265-281.

In some embodiments, the vectors disclosed herein can comprise at least the following, from 5′ to 3′: an RNA polymerase promoter; a polynucleotide sequence encoding a 5′ UTR; a polynucleotide sequence encoding an ORF; a polynucleotide sequence encoding a 3′ UTR; and a polynucleotide sequence encoding at least one RNA aptamer. In some embodiments, the vectors disclosed herein also comprise a polynucleotide sequence encoding a polyA sequence and/or a polyadenylation signal.

A variety of RNA polymerase promoters are known in the art. In some embodiments, the promoter can be a T7 RNA polymerase promoter. Other useful promoters include, but are not limited to, T3 and SP6 RNA polymerase promoters. Consensus nucleotide sequences for T7, T3 and SP6 promoters are known in the art.

Also disclosed herein are host cells (e.g., mammalian cells, e.g., human cells) comprising the vectors or RNA compositions disclosed herein.

Polynucleotides can be introduced into target cells using any of a number of different methods, for instance, commercially available methods which include, but are not limited to, electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendorf, Hamburg Germany), cationic liposome mediated transfection using lipofection, polymer encapsulation, peptide mediated transfection, biolistic particle delivery systems such as “gene guns” (see, for example, Nishikawa, et al. (2001). Hum Gene Ther. 12 (8): 861-70, or the TransIT-RNA transfection Kit (Mirus, Madison WI).

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Regardless of the method used to introduce exogenous nucleic acids into a host cell or otherwise expose a cell to the inhibitor of the present disclosure, in order to confirm the presence of the mRNA sequence in the host cell, a variety of assays may be performed.

### VIII. Self-Replicating RNA, Trans-Replicating RNA and Non-Replicating RNA

### Self-Replicating RNA:

Self-replicating (or self-amplifying) RNA can be produced by using replication elements derived from, e.g., alphaviruses, and substituting the structural viral proteins with a nucleotide sequence encoding a protein of interest (e.g., an antigenic prokaryotic polypeptide). A self-replicating RNA is typically a positive-strand molecule which can be directly translated after delivery to a cell, and this translation provides an RNA-dependent RNA polymerase which then produces both antisense and sense transcripts from the delivered RNA. Thus, the delivered RNA leads to the production of multiple daughter RNAs. These daughter RNAs, as well as collinear subgenomic transcripts, may be translated themselves to provide in situ expression of an encoded antigen, or may be transcribed to provide further transcripts with the same sense as the delivered RNA which are translated to provide in situ expression of the antigen. The overall result of this sequence of transcriptions is a large amplification in the number of the introduced replicon RNAs and so the encoded antigen becomes a major polypeptide product of the cells.

One suitable system for achieving self-replication in this manner is to use an alphavirus-based replicon. These replicons are positive stranded (positive sense-stranded) RNAs which lead to translation of a replicase (or replicase-transcriptase) after delivery to a cell. The replicase is translated as a polyprotein which auto-cleaves to provide a replication complex which creates genomic-strand copies of the positive-strand delivered RNA. These negative (−)-stranded transcripts can themselves be transcribed to give further copies of the positive-stranded parent RNA and also to give a subgenomic transcript which encodes the antigen. Translation of the subgenomic transcript thus leads to in situ expression of the antigen by the infected cell. Suitable alphavirus replicons can use a replicase from a Sindbis virus, a Semliki forest virus, an eastern equine encephalitis virus, a Venezuelan equine encephalitis virus, etc. Mutant or wild-type virus sequences can be used, e.g., the attenuated TC83 mutant of VEEV has been used in replicons, see the following reference: WO2005/113782, incorporated herein by reference.

In one embodiment, each self-replicating RNA described herein encodes (i) an RNA-dependent RNA polymerase which can transcribe RNA from the self-replicating RNA molecule and (ii) an influenza protein antigen. The polymerase can be an alphavirus replicase, e.g., comprising one or more of alphavirus proteins nsP1, nsP2, nsP3, and nsP4. Whereas natural alphavirus genomes encode structural virion proteins in addition to the non-structural replicase polyprotein, in certain embodiments, the self-replicating RNA molecules do not encode alphavirus structural proteins. Thus, the self-replicating RNA can lead to the production of genomic RNA copies of itself in a cell, but not to the production of RNA-containing virions. The inability to produce these virions means that, unlike a wild-type alphavirus, the self-replicating RNA molecule cannot perpetuate itself in infectious form. The alphavirus structural proteins which are necessary for perpetuation in wild-type viruses are absent from self-replicating RNAs of the present disclosure and their place is taken by gene(s) encoding the immunogen of interest, such that the subgenomic transcript encodes the immunogen rather than the structural alphavirus virion proteins. Self-replicating RNA are described in further detail in WO2011005799, incorporated herein by reference.

### Trans-Replicating RNA:

Trans-replicating (or trans-amplifying) RNA possess similar elements as the self-replicating RNA described above. However, with trans replicating RNA, two separate RNA molecules are used. A first RNA molecule encodes for the RNA replicase described above (e.g., the alphavirus replicase) and a second RNA molecule encodes for the protein of interest (e.g., an antigenic prokaryotic polypeptide). The RNA replicase may replicate one or both of the first and second RNA molecule, thereby greatly increasing the copy number of RNA molecules encoding the protein of interest. Trans replicating RNA are described in further detail in WO2017162265, incorporated herein by reference.

### Non-Replicating RNA:

Non-replicating (or non-amplifying) RNA is an RNA without the ability to replicate itself.

### IX. Pharmaceutical Compositions

The pharmaceutical compositions according to this disclosure typically include a nucleic acid, in particular RNA, and more particularly mRNA, and a pharmaceutically acceptable carrier, or a pharmaceutically acceptable excipient or a pharmaceutically acceptable diluent, which makes the composition especially suitable for therapeutic use. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The pharmaceutical composition may for instance be an immunogenic composition, i.e. a composition which, when administered to a subject, elicits an immune response. It should be understood that the terms “immunogenic composition”, “vaccine composition” and “vaccine” are used interchangeably herein and are thus meant to have equivalent meanings.

A pharmaceutical composition of the disclosure can also include one or more additional components such as small molecule immunopotentiators (e.g., TLR agonists). A pharmaceutical composition of the disclosure can also include a delivery system for the RNA, such as a liposome, an oil-in-water emulsion, or a microparticle. In some embodiments, the pharmaceutical composition comprises a lipid nanoparticle (LNP). In one embodiment, the composition comprises an antigen-encoding nucleic acid molecule encapsulated within an LNP.

### X. Methods of Vaccination

The Lyme disease vaccine disclosed herein may be administered to a subject to induce an immune response directed against an antigenic protein from Borrelia, such as the OspA protein expressed on the surface of bacteria of the Borrelia genus, wherein an anti-antigen antibody titer in the subject is increased following vaccination relative to an anti-antigen antibody titer in a subject that is not vaccinated with the Lyme disease vaccine disclosed herein, or relative to an alternative vaccine against Lyme disease. An “anti-antigen antibody” is a serum antibody that binds specifically to the antigen.

In one aspect, the disclosure provides a method of eliciting an immune response, preferably a humoral immune response, and/or of treating or preventing Lyme disease in a subject in need thereof, comprising administering the Lyme disease vaccine disclosed herein to the subject.

The disclosure also provides a Lyme disease vaccine described herein for use in eliciting an immune response, preferably a humoral immune response, and/or in treating or preventing Lyme disease in a subject in need thereof.

The disclosure also provides a Lyme disease vaccine described herein for use in the manufacture of a medicament for use in eliciting an immune response, preferably a humoral immune response, and/or in treating or preventing Lyme disease in a subject in need thereof.

In certain embodiments, the subject has a similar or higher serum concentration of antibodies against OspA after administration of the Lyme disease vaccine, relative to a subject that is administered a Lyme disease vaccine comprising an OspA recombinant protein [Recombitek, OspA fusion ST1-ST2, OspA-ferritin].

The present invention comprises the following embodiments.

Embodiment 1. A nucleic acid comprising an open reading frame (ORF) encoding at least one antigenic polypeptide derived from at least one bacteria of the genus Borrelia, preferably selected from the species B. burgdorferi, afzelii, garinii, bavariensis, mayonii, spielmanii, lusitaniae, bissettii and/or valaisiana, or any strain or isolate thereof.

Embodiment 2. The nucleic acid of embodiment 1, wherein the at least one antigenic polypeptide comprises at least one lipoprotein of Borrelia.

Embodiment 3. The nucleic acid of embodiment 1 or 2, wherein the at least one antigenic polypeptide or lipoprotein is OspA or a fragment or variant thereof, preferably comprising at least 5, 6, 7, 8, 9, 10, 11, 12, 13, 14, 15, 16, 17, 18, 19, 20, 21, 22, 23, 24, 25, 30, 35, 40, 45, 50, 60, 70, 80, 90, or 100 amino acids.

Embodiment 4. The nucleic acid of any one of embodiments 1-3, wherein OspA is derived from OspA serotype (ST) 1, 2, 3, 4, 5, 6, and/or 7, preferably from Borrelia burgdorferi strain B31 of Serotype 1, Borrelia afzelii strain PKO of Serotype 2, Borrelia garinii strain PBr of Serotype 3, Borrelia bavariensis of Serotype 4, Borrelia garinii of Serotype 5, Borrelia garinii of serotype 6, or Borrelia garinii of Serotype 7.

Embodiment 5. The nucleic acid of any one of embodiments 1-4, wherein the at least one OspA polypeptide comprises an amino acid sequence with at least 85%, 86%, 87%, 88%, 89%, 90%, 91%, 92%, 93%, 94%, 95%, 96%, 97%, 98%, 99%, or 100% identity to any one of SEQ ID NOs: 1-7.

Embodiment 6. The nucleic acid of any one of embodiments 1-5, which comprises a nucleotide sequence that is at least 85%, 86%, 87%, 88%, 89%, 90%, 91%, 92%, 93%, 94%, 95%, 96%, 97%, 98%, 99%, or 100% identical to any one of SEQ ID NOs: 10-13 and 16-19.

Embodiment 7. The nucleic acid of any one of embodiments 1-6, which encodes at least two different OspA serotypes or fragments or variants thereof, wherein each of the at least two different OspA serotypes or fragments or variants thereof preferably comprises at least 5, 6, 7, 8, 9, 10, 11, 12, 13, 14, 15, 16, 17, 18, 19, 20, 21, 22, 23, 24, 25, 30, 35, 40, 45, 50, 60, 70, 80, 90, or 100 amino acids.

Embodiment 8. The nucleic acid of embodiment 7, wherein the OspA of one serotype or fragment or variant thereof is fused to a OspA of a different serotype, or fragment or variant thereof.

Embodiment 9. The nucleic acid of embodiment 8, wherein the fused OspA of different serotypes, or fragments or variants thereof are separated by a linker sequence, wherein the linker sequence is preferably derived from P66 or comprises an amino acid sequence with at least 85%, 86%, 87%, 88%, 89%, 90%, 91%, 92%, 93%, 94%, 95%, 96%, 97%, 98%, 99%, or 100% identity to SEQ ID NO: 8 or to SEQ ID NO: 9.

Embodiment 10. The nucleic acid of any one of embodiments 1-9, wherein the nucleic acid is non-replicating nucleic acid.

Embodiment 11. The nucleic acid of any one of embodiments 1-9, wherein the nucleic acid is self-replicating or trans-replicating nucleic acid.

Embodiment 12. The nucleic acid of any one of embodiments 1-11, wherein the nucleic acid is DNA.

Embodiment 13. The nucleic acid of any one of embodiments 1-11, wherein the nucleic acid is messenger RNA (mRNA).

Embodiment 14. The nucleic acid of embodiment 13, wherein the mRNA comprises at least one 5′ cap, at least one 5′ untranslated region (5′ UTR), at least one 3′ untranslated region (3′ UTR), and/or at least one polyadenylation (polyA) sequence, wherein for instance the mRNA may comprise:
at least one 5′ cap, or
at least one 5′ UTR, or
at least one 3′ UTR, or
at least one 5′ UTR and at least one 3′UTR, or
at least one polyA sequence, or
at least one 5′ cap and at least one polyA sequence, or
at least one 5′ cap, at least one 5′ UTR, at least one 3′ UTR and at least one polyA sequence.

Embodiment 15. The nucleic acid of embodiment 14, wherein the 5′ cap is selected from the group consisting of 3′-O-Me-m7G(5′)ppp(5′)G (the ARCA cap), G(5′)ppp(5′)A, G(5′)ppp(5′)G, m7G(5′)ppp(5′)A, m7G(5′)ppp(5′)G, m7G(5′)ppp(5′)(2′OMeA)pG, m7G(5′)ppp(5′)(2′OMeA)pU, and m7G(5′)ppp(5′)(2′OMeG)pG.

Embodiment 16. The nucleic acid of embodiment 14, wherein the 5′ cap comprises:

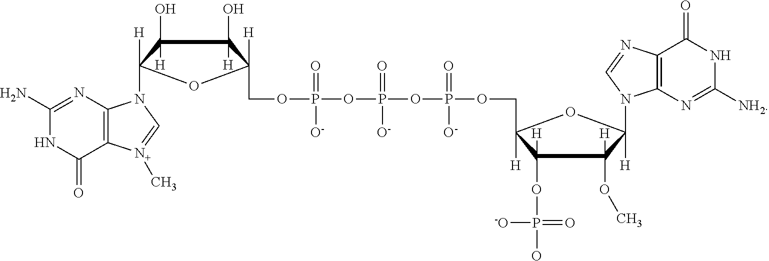

Embodiment 17. The nucleic acid of any of embodiments 14-16, wherein the 5′ UTR is about 10 to 5,000 nucleotides in length (e.g., about 50 to 500 nucleotides in length or at least about 10 nucleotides in length, about 20 nucleotides in length, about 30 nucleotides in length, about 40 nucleotides in length, about 50 nucleotides in length, about 100 nucleotides in length, about 150 nucleotides in length, about 200 nucleotides in length, about 250 nucleotides in length, about 300 nucleotides in length, about 350 nucleotides in length, about 400 nucleotides in length, about 450 nucleotides in length, about 500 nucleotides in length, about 550 nucleotides in length, about 600 nucleotides in length, about 650 nucleotides in length, about 700 nucleotides in length, about 750 nucleotides in length, about 800 nucleotides in length, about 850 nucleotides in length, about 900 nucleotides in length, about 950 nucleotides in length, about 1,000 nucleotides in length, about 1,500 nucleotides in length, about 2,000 nucleotides in length, about 2,500 nucleotides in length, about 3,000 nucleotides in length, about 3,500 nucleotides in length, about 4,000 nucleotides in length, about 4,500 nucleotides in length or about 5,000 nucleotides in length).

Embodiment 18. The nucleic acid of any of embodiments 14-17, wherein the 3′ UTR is 50 to 5,000 nucleotides in length or longer (e.g., 50 to 1,000 nucleotides in length or longer or about 50 nucleotides in length, about 100 nucleotides in length, about 150 nucleotides in length, about 200 nucleotides in length, about 250 nucleotides in length, about 300 nucleotides in length, about 350 nucleotides in length, about 400 nucleotides in length, about 450 nucleotides in length, about 500 nucleotides in length, about 550 nucleotides in length, about 600 nucleotides in length, about 650 nucleotides in length, about 700 nucleotides in length, about 750 nucleotides in length, about 800 nucleotides in length, about 850 nucleotides in length, about 900 nucleotides in length, about 950 nucleotides in length, about 1,000 nucleotides in length, about 1,500 nucleotides in length, about 2,000 nucleotides in length, about 2,500 nucleotides in length, about 3,000 nucleotides in length, about 3,500 nucleotides in length, about 4,000 nucleotides in length, about 4,500 nucleotides in length, or about 5,000 nucleotides in length).

Embodiment 19. The nucleic acid of any of embodiments 14-18, wherein the 5′ and/or 3′ UTR is derived from a gene distinct from the one encoded by the mRNA transcript (i.e., the UTR is a heterologous UTR).

Embodiment 20. The nucleic acid of any of embodiments 14-19, wherein the 5′ and/or 3′ UTR sequences are derived from mRNA which are stable (e.g., globin, actin, GAPDH, tubulin, histone, or citric acid cycle enzymes) to increase the stability of the mRNA.

Embodiment 21. The nucleic acid of any of embodiments 14-19, wherein the 5′ UTR is derived from a CMV immediate-early 1 (IE1) gene.

Embodiment 22. The nucleic acid of any of embodiments 14-19, wherein the 5′ UTR comprises the sequence GGGAUCCUACC (SEQ ID NO: 20).

Embodiment 23. The nucleic acid of any of embodiments 14-19, wherein the 5′ UTR is derived from the 5′ UTR of a TOP gene.

Embodiment 24. The nucleic acid of any of embodiments 14-19, wherein the 5′ UTR is derived from a ribosomal protein Large 32 (L32) gene.

Embodiment 25. The nucleic acid of any of embodiments 14-19, wherein the 5′ UTR is derived from the 5′ UTR of an hydroxysteroid (17-b) dehydrogenase 4 gene (HSD17B4).

Embodiment 26. The nucleic acid of any of embodiments 14-19, wherein the 5′ UTR is derived from the 5′ UTR of an ATP5A1 gene.

Embodiment 27. The nucleic acid of any of embodiments 14-19, wherein an internal ribosome entry site (IRES) is used instead of a 5′ UTR.

Embodiment 28. The nucleic acid of any of embodiments 14-19, wherein the 5′ UTR comprises a nucleic acid sequence set forth in SEQ ID NO: 14.

Embodiment 29. The nucleic acid of any of embodiments 14-28, wherein the 3′ UTR comprises a nucleic acid sequence set forth in SEQ ID NO: 15.

Embodiment 30. The nucleic acid of any of embodiments 14-29, wherein the at least one polyadenylation (polyA) sequence comprises about 10 to about 500 adenosine nucleotides, about 10 to about 200 adenosine nucleotides, about 40 to about 200 adenosine nucleotides, or about 40 to about 150 adenosine nucleotides.

Embodiment 31. The nucleic acid of any of embodiments 14-30, wherein the at least one polyadenylation (polyA) sequence comprises at least about 10, 50, 75, 100, 150, 200, 250, 300, 350, 400, 450, or 500 adenosine nucleotides.

Embodiment 32. The nucleic acid of any of embodiments 14-31, wherein the at least one polyadenylation (polyA) sequence comprises the sequence

(SEQ ID NO: 21)
AAAAAAAAAAAAAAAAAAAAAAAAAAAAAAGCAUAUGACUAAAAAAAAA
AAAAAAAAAAAAAAAAAAAAAAAAAAAAAAAAAAAAAAAAAAAAAAAAA
AAAAAAAAAAAA.

Embodiment 33. The nucleic acid of any of embodiments 30-32, wherein the 5′ UTR is about 10 to 5,000 nucleotides in length (e.g., about 50 to 500 nucleotides in length or at least about 10 nucleotides in length, about 20 nucleotides in length, about 30 nucleotides in length, about 40 nucleotides in length, about 50 nucleotides in length, about 100 nucleotides in length, about 150 nucleotides in length, about 200 nucleotides in length, about 250 nucleotides in length, about 300 nucleotides in length, about 350 nucleotides in length, about 400 nucleotides in length, about 450 nucleotides in length, about 500 nucleotides in length, about 550 nucleotides in length, about 600 nucleotides in length, about 650 nucleotides in length, about 700 nucleotides in length, about 750 nucleotides in length, about 800 nucleotides in length, about 850 nucleotides in length, about 900 nucleotides in length, about 950 nucleotides in length, about 1,000 nucleotides in length, about 1,500 nucleotides in length, about 2,000 nucleotides in length, about 2,500 nucleotides in length, about 3,000 nucleotides in length, about 3,500 nucleotides in length, about 4,000 nucleotides in length, about 4,500 nucleotides in length or about 5,000 nucleotides in length).

Embodiment 34. The nucleic acid of any of embodiments 30-33, wherein the 3′ UTR is 50 to 5,000 nucleotides in length or longer (e.g., 50 to 1,000 nucleotides in length or longer or about 50 nucleotides in length, about 100 nucleotides in length, about 150 nucleotides in length, about 200 nucleotides in length, about 250 nucleotides in length, about 300 nucleotides in length, about 350 nucleotides in length, about 400 nucleotides in length, about 450 nucleotides in length, about 500 nucleotides in length, about 550 nucleotides in length, about 600 nucleotides in length, about 650 nucleotides in length, about 700 nucleotides in length, about 750 nucleotides in length, about 800 nucleotides in length, about 850 nucleotides in length, about 900 nucleotides in length, about 950 nucleotides in length, about 1,000 nucleotides in length, about 1,500 nucleotides in length, about 2,000 nucleotides in length, about 2,500 nucleotides in length, about 3,000 nucleotides in length, about 3,500 nucleotides in length, about 4,000 nucleotides in length, about 4,500 nucleotides in length, or about 5,000 nucleotides in length).

Embodiment 35. The nucleic acid of any of embodiments 30-34, wherein the 5′ and/or 3′ UTR is derived from a gene distinct from the one encoded by the mRNA transcript (i.e., the UTR is a heterologous UTR).

Embodiment 36. The nucleic acid of any of embodiments 30-35, wherein the 5′ and/or 3′ UTR sequences are derived from mRNA which are stable (e.g., globin, actin, GAPDH, tubulin, histone, or citric acid cycle enzymes) to increase the stability of the mRNA.

Embodiment 37. The nucleic acid of any of embodiments 30-35, wherein the 5′ UTR is derived from a CMV immediate-early 1 (IE1) gene.

Embodiment 38. The nucleic acid of any of embodiments 30-35, wherein the 5′ UTR comprises the sequence GGGAUCCUACC (SEQ ID NO: 20).

Embodiment 39. The nucleic acid of any of embodiments 30-35, wherein the 5′ UTR is derived from the 5′ UTR of a TOP gene.

Embodiment 40. The nucleic acid of any of embodiments 30-35, wherein the 5′ UTR is derived from a ribosomal protein Large 32 (L32) gene.

Embodiment 41. The nucleic acid of any of embodiments 30-35, wherein the 5′ UTR is derived from the 5′ UTR of an hydroxysteroid (17-b) dehydrogenase 4 gene (HSD17B4).

Embodiment 42. The nucleic acid of any of embodiments 30-35, wherein the 5′ UTR is derived from the 5′ UTR of an ATP5A1 gene.

Embodiment 43. The nucleic acid of any of embodiments 30-35, wherein an internal ribosome entry site (IRES) is used instead of a 5′ UTR.

Embodiment 44. The nucleic acid of any of embodiments 30-35, wherein the 5′ UTR comprises a nucleic acid sequence set forth in SEQ ID NO: 14.

Embodiment 45. The nucleic acid of any of embodiments 30-35, wherein the 3′ UTR comprises a nucleic acid sequence set forth in SEQ ID NO: 15.

Embodiment 46. The nucleic acid of any of embodiments 13-45, wherein the mRNA comprises at least the following structural elements:
(i) a 5′ cap with the following structure:

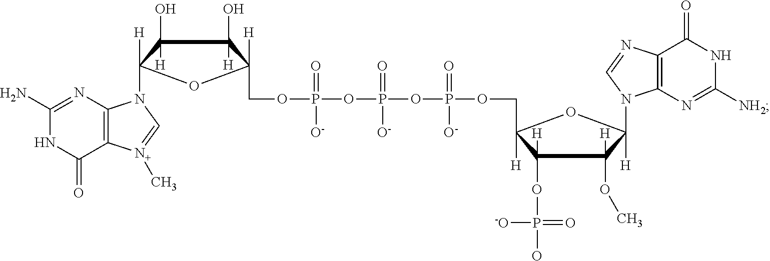

(ii) a 5′ untranslated region (5′ UTR) having the nucleic acid sequence of SEQ ID NO: 14;
(iii) at least one OspA open reading frame having any one of the nucleic acid sequences of SEQ ID NOs: 16-19;
(iv) a 3′ untranslated region (3′ UTR) having the nucleic acid sequence of SEQ ID NO: 15; and
(v) a polyA tail.

Embodiment 47. A nucleic acid, wherein the nucleic acid is a mRNA comprising an open reading frame (ORF) encoding at least one antigenic polypeptide derived from at least one bacteria of the genus Borrelia, wherein the mRNA comprises at least of the following structural elements:
(i) a 5′ cap with the following structure:

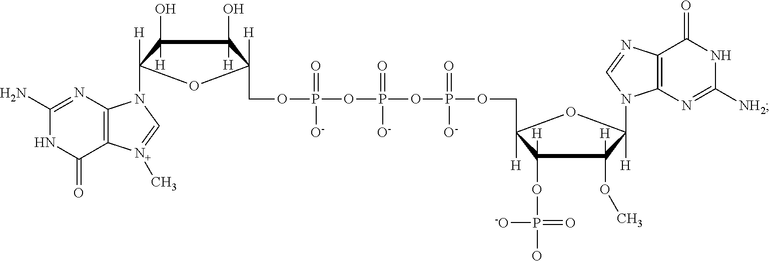

(ii) a 5′ untranslated region (5′ UTR) having the nucleic acid sequence of SEQ ID NO: 14;
(iii) at least one OspA open reading frame having any one of the nucleic acid sequences of SEQ ID NOs: 16-19;
(iv) a 3′ untranslated region (3′ UTR) having the nucleic acid sequence of SEQ ID NO: 15; and
(v) a polyA tail;
   wherein the mRNA is formulated in a lipid nanoparticle (LNP) comprising:
cKK-E10 at a molar ratio of 40%,
DMG-PEG2000 at a molar ratio of 1.5%,
cholesterol at a molar ratio of 28.5%, and
DOPE at a molar ratio of 30%; or
OF-02 at a molar ratio of 40%;
DMG-PEG2000 at a molar ratio of 1.5%;
cholesterol at a molar ratio of 28.5%; and
DOPE at a molar ratio of 30%; or
GL-HEPES-E3-E10-DS-3-E18-1 at a molar ratio of 40%;
DMG-PEG2000 at a molar ratio of 1.5%;
cholesterol at a molar ratio of 28.5%; and
DOPE at a molar ratio of 30%; or
GL-HEPES-E3-E12-DS-4-E10 at a molar ratio of 40%;
DMG-PEG2000 at a molar ratio of 1.5%;
cholesterol at a molar ratio of 28.5%; and
DOPE at a molar ratio of 30%; or
GL-HEPES-E3-E12-DS-3-E14 at a molar ratio of 40%;
DMG-PEG2000 at a molar ratio of 1.5%;
cholesterol at a molar ratio of 28.5%; and
DOPE at a molar ratio of 30%; or
9-heptadecanyl 8-{(2-hydroxyethyl)[6-oxo-6-(undecyloxy) hexyl]amino}octanoate (SM-102) at a molar ratio of 50%;
1,2-distearoyl-sn-glycero-3-phosphocholine (DSPC) at a molar ratio of 10%;
cholesterol at a molar ratio of 38.5%; and
1,2-dimyristoyl-rac-glycero-3-methoxypolyethylene glycol-2000 (DMG-PEG2000) at a molar ratio of 1.5%; or
(4-hydroxybutyl)azanediyl]di(hexane-6,1-diyl) bis(2-hexyldecanoate) (ALC-0315) at a molar ratio of 46.3%;
1,2-distearoyl-sn-glycero-3-phosphocholine (DSPC) at a molar ratio of 9.4%;
cholesterol at a molar ratio of 42.7%; and
2-[(polyethylene glycol)-2000]-N,N-ditetradecylacetamide (ALC-0159) at a molar ratio of 1.6%; or
(4-hydroxybutyl)azanediyl]di(hexane-6,1-diyl) bis(2-hexyldecanoate) (ALC-0315) at a molar ratio of 47.4%;
1,2-distearoyl-sn-glycero-3-phosphocholine (DSPC) at a molar ratio of 10%;
cholesterol at a molar ratio of 40.9%; and
2-[(polyethylene glycol)-2000]-N,N-ditetradecylacetamide (ALC-0159) at a molar ratio of 1.7%.

Embodiment 48. The nucleic acid of any one of embodiments 1-47, which comprises at least one chemical modification.

Embodiment 49. The nucleic acid of embodiment 48, wherein at least 20%, at least 30%, at least 40%, at least 50%, at least 60%, at least 70%, at least 80%, at least 85%, at least 90%, at least 95%, or 100% of the uracil nucleotides are chemically modified.

Embodiment 50. The nucleic acid of embodiment 48 or 49, wherein the chemical modification is selected from the group consisting of pseudouridine, N1-methylpseudouridine, 2-thiouridine, 4′-thiouridine, 5-methylcytidine, 2-thio-I-methyl-1-deaza-pseudouridine, 2-thio-I-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-I-methyl-pseudouridine, 4-thio-pseudouridine, 5-aza-uridine, dihydropseudouridine, 5-methyluridine, 5-methyluridine, 5-methoxyuridine, and 2′-O-methyl uridine.

Embodiment 51. The nucleic acid of any one of embodiments 48-50, wherein the chemical modification is selected from the group consisting of pseudouridine, N1-methylpseudouridine, 5-methylcytidine, 5-methoxyuridine, and a combination thereof.

Embodiment 52. The nucleic acid of any one of embodiments 48-51, wherein the chemical modification is N1-methylpseudouridine.

Embodiment 53. A composition comprising at least one nucleic acid of any one of embodiments 1-52.

Embodiment 54. The composition of embodiment 53, wherein the nucleic acid is formulated in a non-viral delivery system.

Embodiment 55. The composition of embodiment 53 or 54, which comprises a lipid nanoparticle (LNP).

Embodiment 56. The composition of embodiment 55, wherein the nucleic acid is encapsulated in the LNP.

Embodiment 57. The composition of embodiment 55 or 56, wherein the LNP comprises at least one cationic lipid, wherein the cationic lipid may be biodegradable or not biodegradable, cleavable or not cleavable, and wherein the cationic lipid is preferably selected from the group consisting of cKK-E10; OF-02; [(6Z,9Z,28Z,31Z)-heptatriaconta-6,9,28,31-tetraen-19-yl] 4-(dimethylamino) butanoate (D-Lin-MC3-DMA); 2,2-dilinoleyl-4-dimethylaminoethyl-[1,3]-dioxolane (DLin-KC2-DMA); 1,2-dilinoleyloxy-N,N-dimethyl-3-aminopropane (DLin-DMA); di((Z)-non-2-en-1-yl) 9-((4-(dimethylamino) butanoyl)oxy) heptadecanedioate (L319); 9-heptadecanyl 8-{(2-hydroxyethyl) [6-oxo-6-(undecyloxy) hexyl]amino}octanoate (SM-102); [(4-hydroxybutyl) azanediyl]di(hexane-6,1-diyl) bis(2-hexyldecanoate) (ALC-0315); [3-(dimethylamino)-2-[(Z)-octadec-9-enoyl] oxypropyl] (Z)-octadec-9-enoate (DODAP); 2,5-bis(3-aminopropylamino)-N-[2-[di(heptadecyl)amino]-2-oxoethyl]pentanamide (DOGS); [(3S,8S,9S,10R,13R,14S,17R)-10,13-dimethyl-17-[(2R)-6-methylheptan-2-yl]-2,3,4,7,8,9,11,12,14,15,16,17-dodecahydro-1H-cyclopenta[a]phenanthren-3-yl] N-[2-(dimethylamino)ethyl]carbamate (DC-Chol); tetrakis(8-methylnonyl) 3,3′,3″,3″′-(((methylazanediyl) bis(propane-3,1 diyl))bis (azanetriyl))tetrapropionate (306Oi10); decyl (2-(dioctylammonio)ethyl) phosphate (9A1P9); ethyl 5,5-di((Z)-heptadec-8-en-1-yl)-1-(3-(pyrrolidin-1-yl)propyl)-2,5-dihydro-1H-imidazole-2-carboxylate (A2-Iso5-2DC18); bis(2-(dodecyldisulfanyl)ethyl) 3,3′-((3-methyl-9-oxo-10-oxa-13,14-dithia-3,6-diazahexacosyl)azanediyl)dipropionate (BAME-O16B); 1,1′-((2-(4-(2-((2-(bis(2-hydroxydodecyl)amino)ethyl) (2-hydroxydodecyl)amino)ethyl) piperazin-1-yl)ethyl)azanediyl) bis(dodecan-2-ol) (C12-200); 3,6-bis(4-(bis(2-hydroxydodecyl)amino)butyl)piperazine-2,5-dione (cKK-E12); hexa(octan-3-yl) 9,9′,9″,9″′,9″″,9″″′-((((benzene-1,3,5-tricarbonyl)yris(azanediyl)) tris (propane-3,1-diyl)) tris(azanetriyl))hexanonanoate (FTT5); (((3,6-dioxopiperazine-2,5-diyl)bis(butane-4,1-diyl))bis(azanetriyl))tetrakis(ethane-2,1-diyl) (9Z,9′Z,9″Z,9″′Z, 12Z, 12′Z, 12″Z, 12″′Z)-tetrakis (octadeca-9,12-dienoate) (OF-Deg-Lin); TT3; N¹,N³,N⁵-tris(3-(didodecylamino)propyl)benzene-1,3,5-tricarboxamide; N1-[2-((1S)-1-[(3-aminopropyl)amino]-4-[di(3-aminopropyl)amino]butylcarboxamido)ethyl]-3,4-di[oleyloxy]-benzamide (MVL5); heptadecan-9-yl 8-((2-hydroxyethyl) (8-(nonyloxy)-8-oxooctyl)amino)octanoate (Lipid 5); GL-HEPES-E3-E10-DS-3-E18-1; GL-HEPES-E3-E12-DS-4-E10; GL-HEPES-E3-E12-DS-3-E14; and combinations thereof

Embodiment 58. The composition of embodiment 57, wherein the LNP further comprises a polyethylene glycol (PEG) conjugated (PEGylated) lipid, a cholesterol-based lipid, and/or a helper lipid.

Embodiment 59. The composition of any one of embodiments 55-58, wherein the LNP comprises:—a cationic lipid at a molar ratio of 35% to 55%; —a polyethylene glycol (PEG) conjugated (PEGylated) lipid at a molar ratio of 0.25% to 2.75%; —a cholesterol-based lipid at a molar ratio of 20% to 45%; and—a helper lipid at a molar ratio of 5% to 35%, wherein all of the molar ratios are relative to the total lipid content of the LNP.

Embodiment 60. The composition of any one of embodiments 55-59, wherein the LNP comprises:—a cationic lipid at a molar ratio of 40%; —a PEGylated lipid at a molar ratio of 1.5%; —a cholesterol-based lipid at a molar ratio of 28.5%; and—a helper lipid at a molar ratio of 30%.

Embodiment 61. The composition of any one of embodiments 55-60, wherein the LNP comprises:—a cationic lipid at a molar ratio of 45 to 50%; —a PEGylated lipid at a molar ratio of 1.5 to 1.7%; —a cholesterol-based lipid at a molar ratio of 38 to 43%; and—a helper lipid at a molar ratio of 9 to 10%.

Embodiment 62. The composition of any one of embodiments 58-61, wherein the PEGylated lipid is dimyristoyl-PEG2000 (DMG-PEG2000) or 2-[(polyethylene glycol)-2000]-N,N-ditetradecylacetamide (ALC-0159).

Embodiment 63. The composition of any one of embodiments 58-62, wherein the cholesterol-based lipid is cholesterol.

Embodiment 64. The composition of any one of embodiments 58-63, wherein the helper lipid is 1,2-dioleoyl-SN-glycero-3-phosphoethanolamine (DOPE) or 1,2-distearoyl-sn-glycero-3-phosphocholine (DSPC).

Embodiment 65. The composition of any one of embodiments 55-64, wherein the LNP comprises:—a cationic lipid selected from the group consisting of OF-02, cKK-E10, GL-HEPES-E3-E10-DS-3-E18-1, GL-HEPES-E3-E12-DS-4-E10, and GL-HEPES-E3-E12-DS-3-E14, at a molar ratio of 40%; —DMG-PEG2000 at a molar ratio of 1.5%; —cholesterol at a molar ratio of 28.5%; and—DOPE at a molar ratio of 30%.

Embodiment 66. The composition of any one of embodiments 55-65, wherein the LNP comprises:—SM-102 at a molar ratio of 50%; —DMG-PEG2000 at a molar ratio of 1.5%; —cholesterol at a molar ratio of 38.5%; and—DSPC at a molar ratio of 10%.

Embodiment 67. The composition of any one of embodiments 55-65, wherein the LNP comprises:—ALC-0315 at a molar ratio of 46.3%; —ALC-0159 at a molar ratio of 1.6%; —cholesterol at a molar ratio of 42.7%; and—DSPC at a molar ratio of 9.4%.

Embodiment 68. The composition of any one of embodiments 55-65, wherein the LNP comprises:—ALC-0315 at a molar ratio of 47.4%; —ALC-0159 at a molar ratio of 1.7%; —cholesterol at a molar ratio of 40.9%; and—DSPC at a molar ratio of 10%.

Embodiment 69. The composition of any one of embodiments 55-68, wherein the LNP has an average diameter of 30 nm to 200 nm.

Embodiment 70. The composition of any one of embodiments 55-68, wherein the LNP has an average diameter of 80 nm to 150 nm.

Embodiment 71. The composition of any one of embodiments 55-70, comprising between 1 mg/mL to 10 mg/ml of the LNP.

Embodiment 72. The composition of any one of embodiments 55-71, wherein the LNP comprises between 1 and 20 nucleic acid molecules, preferably mRNA molecules.

Embodiment 73. The composition of any one of embodiments 53-72, which is formulated for administration intramuscularly, intranasally, intravenously, subcutaneously, or intradermally.

Embodiment 74. The composition of any one of embodiments 53-73, wherein the composition comprises a phosphate-buffer saline.

Embodiment 75. The composition of any one of embodiments 53-74, wherein the composition is a pharmaceutical composition, for example an immunogenic composition or a vaccine, in particular a Lyme disease vaccine.

Embodiment 76. The nucleic acid of any one or embodiments 1-52 or the composition of any one of embodiments 53-75 for use in eliciting an immune response, preferably a humoral immune response, and/or in treating or preventing Lyme disease in a subject in need thereof, wherein preferably the subject has a higher serum concentration of antibodies against OspA after administration of the nucleic acid or composition, relative to a subject that is administered a Lyme disease vaccine comprising an OspA recombinant protein vaccine, and/or wherein preferably the subject is a mammal, more preferably a human, a dog, a cat, a llama, a bovine, a sheep, a goat, a horse, a rodent, a mouse, a rat, a rabbit, a monkey, a primate or a pig, even more preferably a human.

Embodiment 77. A method of eliciting an immune response, preferably a humoral immune response, and/or of treating or preventing Lyme disease in a subject in need thereof, comprising administering to the subject, optionally intramuscularly, intranasally, intravenously, subcutaneously, or intradermally, an effective amount of the nucleic acid of any one or embodiments 1-52 or the composition of any one of embodiments 53-75, wherein preferably the subject has a higher serum concentration of antibodies against OspA after administration of the nucleic acid or composition, relative to a subject that is administered a Lyme disease vaccine comprising an OspA recombinant protein vaccine, and/or wherein preferably the subject is a mammal, more preferably a human, a dog, a cat, a llama, a bovine, a sheep, a goat, a horse, a rodent, a mouse, a rat, a rabbit, a monkey, a primate or a pig, even more preferably a human.

Embodiment 78. Use of the nucleic acid of any one or embodiments 1-52 or of the composition of any one of embodiments 53-75 for the manufacture of a medicament for use in eliciting an immune response, preferably a humoral immune response, and/or in treating or preventing Lyme disease, in a subject in need thereof, wherein preferably the subject has a higher serum concentration of antibodies against OspA after administration of the nucleic acid or composition, relative to a subject that is administered a Lyme disease vaccine comprising an OspA recombinant protein vaccine, and/or wherein preferably the subject is a mammal, more preferably a human, a dog, a cat, a llama, a bovine, a sheep, a goat, a horse, a rodent, a mouse, a rat, a rabbit, a monkey, a primate or a pig, even more preferably a human.

In order that this disclosure may be better understood, the following examples are set forth. These examples are for purposes of illustration only and are not to be construed as limiting the scope of the disclosure in any manner.

### EXAMPLES

The foregoing description of the specific embodiments will so fully reveal the general nature of the disclosure that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

### Example 1: Materials & Methods

mRNA-OspA Production

mRNAs were produced as previously published (Kalnin et al (2021), NPJ Vaccines 6(1): 61 and WO2021226436). Briefly, mRNAs incorporating coding sequences containing either the OspA ST1 or ST2 were synthesized by in vitro transcription employing RNA polymerase with a plasmid DNA template encoding the desired gene using unmodified nucleotides. The resulting purified precursor mRNA was reacted further via enzymatic addition of a 5′ cap structure (Cap 1) and a 3′ poly(A) tail of approximately 200 nucleotides in length as determined by gel electrophoresis.

For the preparation of mRNA/lipid nanoparticle (LNP) formulations, an ethanolic solution of a mixture of lipids (cationic/ionizable lipid, phosphatidylethanolamine, cholesterol and polyethylene glycol-lipid) at a fixed lipid and mRNA ratio were combined with an aqueous buffered solution of target mRNA at an acidic pH under controlled conditions to yield a suspension of uniform LNPs. Upon ultrafiltration and diafiltration into a suitable diluent system, the resulting nanoparticle suspensions were diluted to final concentration, filtered, and stored frozen at −80° C. until use.

### Production of Antigens Used as Benchmark Comparators

OspA-ferritin ST1 and ST2 antigens were produced by Sanofi Breakthrough Lab in Cambridge, MA, USA according to the Material and Method previously published (Kamp et al (2020), NPJ Vaccines 5(1): 33).

For OspA fusion ST1-ST2, the in-house plasmid pSP401+LPP-chimer OspA1-OspA2, allowing the expression of the OspA fusion ST1-ST2 C-terminus domains, was introduced into the E. coli expression strain C43-(DE3) (Lucigen). After 2 to 3 hours of growth at 37° C. in a rich medium, the expression of the protein of interest was induced by the addition of an inducer and the culture was stopped 3 hours post-induction. After processing the bacterial pellets, the protein was visualized on SDS-Page gel stained Coomassie blue or by Western Blot using a specific antibody. The scale-up was done on the best expression conditions to produce enough biomass necessary for purification. Bacterial pellets were treated with lysozyme to extract membrane proteins. OspA1-OspA2 fusion protein was then extracted with Urea 2M+Triton X114 2%. After three incubation-centrifugation steps at 37° C., the lower phase was collected and subjected to a Q Sepharose chromatography in presence of Zwittergent 3.14 detergent (0.5%). The fractions eluted with 400 mM NaCl were subjected to ceramic hydroxyapatite chromatography. OspA1-OspA2 fusion protein was eluted with Tween 0.05% PO4 NaNa2 180 mM pH 6,7 buffer and substituted to PBS+Tween 20 0.05% pH 7.3 as final buffer.

### Antigens and Mouse Immunizations

OF-1 mice (Charles River) were randomized into immunization groups of eight animals each. Four different doses of mRNA-OspA-LNP were administered intramuscularly (50 μL) at day 0 (D0) (dose 1) and day 21 (D21) (dose 2): 0.2 μg, 1 μg, 5 μg or 10 μg. Sera were taken at baseline (D0), day 20 (D20), and day 35 (D35).

Two mRNA-OspA sequences were tested: mRNA-OspA-ST1-Native and mRNA-OspA-ST2-Native.

mRNA formulations were compared to negative control LNP alone and to the benchmark Lyme dog vaccine RECOMBITEK® (Merial) at 1 μg/dose (50 μL).

### OspA-Specific IgG ELISA

The antibody response in mice was determined by ELISA. Briefly, 384-well microplates (Perkin Elmer #6007509) were coated with 1 μg/mL of OspA-His of the determined serotype (ST1 or ST2) diluted in PBS and incubated overnight at 4° C. The OspA-His was removed and the plates were blocked with 5% skim milk dissolved in PBS-tween. After removing the blocking reagent, the primary serum samples were added after being serially diluted 2-fold in 1% skim milk-PBS-Tween. After a 1.5 hours incubation at room temperature, with the primary serum samples, the plates were washed with PBS-Tween and incubated with Goat anti-mouse IgG-HRP (Jackson 115-036-062) for 1.5 h at room temperature. The secondary antibody was aspirated and washed, and the plates were incubated with TMB substrate (TEBU-TMB100-1000) followed by equal volume of stop solution (HCl 1N). Absorbance was measured at 450 nm-650 nm. OspA-specific IgG titers were quantified through an internal anti-OspA mouse serum reference. The titer of this reference was previously calculated as the reciprocal dilution to obtain an OD of 1.

mRNA Encoding OspA Protein

The causative agent of Lyme disease are bacteria of the Borrelia genus. Four species from the Borrelia genus cause most human disease: B. burgdorferi, B. afzelii, B. garinii and B. bavariensis. Each Borrelia species has surface expression of the Outer surface protein A (OspA), and seven OspA serotypes (ST1-ST7) are particularly prevalent in U.S. and Europe, with ST1 representing approximately 98% of the U.S. OspA serotypes, while ST2 represents over 50% of the European OspA serotypes. To that end, mRNA expressing either OspA ST1 or ST2 were designed.

OspA native sequence was used. Each OspA polypeptide sequence recited below lacks an N-terminal methionine, which is typically removed in eukaryotic cells. In certain embodiments, the amino acid sequence set forth in any one of SEQ ID NOs: 1-7 further comprises an N-terminal methionine amino acid.

Each nucleic acid recited in Table 3 corresponds to the mRNA sequence. A corresponding DNA sequence may be used as the template to generate mRNA through in vitro transcription. The DNA sequence is identical to the mRNA sequence except for the substitution of U nucleotides in the mRNA sequence to T nucleotides.

Amino acid and nucleic acid sequences of the OspA proteins and nucleic acid sequences encoding the same are recited below in Table 2 and Table 3 (as well as amino acid sequences for linkers and nucleic acid sequences of 5′ UTR and 3′ UTR).

TABLE 3
Amino acid sequences of OspA proteins and linkers

| SEQ ID NO/ | |
|---|---|
| Description | Amino Acid sequence |
| SEQ ID NO: | KQNVSSLDEKNSVSVDLPGEMKVLVSKEKNKDGKYDLIATVDKLELKGTSDK |
| 1: | NNGSGVLEGVKADKSKVKLTISDDLGQTTLEVFKEDGKTLVSKKVTSKDKSST |
| OspA ST1 | EEKFNEKGEVSEKIITRADGTRLEYTGIKSDGSGKAKEVLKGYDLKGELSSEK |
| Native | TTLVVKEGTVTLSKNISKSGEVSVELNDTDSSAATKKTAAWNSGTSTLTITVNS |
| | KKTKDLVFTKENTITVQQYDSNGTKLEGSAVEITKLDEIKNALK |
| SEQ ID NO: | KQNVSSLDEKNSASVDLPGEMKVLVSKEKDKDGKYSLKATVDKIELKGTSDK |
| 2: | DNGSGVLEGTKDDKSKAKLTIADDLSKTTFELFKEDGKTLVSRKVSSKDKTST |
| OspA ST2 | DEMFNEKGELSAKTMTRENGTKLEYTEMKSDGTGKAKEVLKNFTLEGKVAN |
| Native | DKVTLEVKEGTVTLSKEIAKSGEVTVALNDTNTTQATKKTGAWDSKTSTLTISV |
| | NSKKTTQLVFTKQDTITVQKYDSAGTNLEGTAVEIKTLDELKNALK |
| SEQ ID NO: | KQNVSSLDEKNSVSVDLPGGMKVLVSKEKDKDGKYSLMATVEKLELKGTSDK |
| 3: | SNGSGVLEGEKADKSKAKLTISQDLNQTTFEIFKEDGKTLVSRKVNSKDKSST |
| OspA ST3 | EEKFNDKGKLSEKVVTRANGTRLEYTEIKNDGSGKAKEVLKGFALEGTLTDG |
| Native | GETKLTVTEGTVTLSKNISKSGEITVALNDTETTPADKKTGEWKSDTSTLTISK |
| | NSQKPKQLVFTKENTITVQNYNRAGNALEGSPAEIKDLAELKAALK |
| SEQ ID NO: | KQNVSSLDEKNSVSVDLPGEMKVLVSKEKDKDGKYSLMATVDKLELKGTSDK |
| 4: | SNGSGTLEGEKSDKSKAKLTISEDLSKTTFEIFKEDGKTLVSKKVNSKDKSSIE |
| OspA ST4 | EKFNAKGELSEKTILRANGTRLEYTEIKSDGTGKAKEVLKDFALEGTLAADKTT |
| Native | LKVTEGTVVLSKHIPNSGEITVELNDSNSTQATKKTGKWDSNTSTLTISVNSKK |
| | TKNIVFTKEDTITVQKYDSAGTNLEGNAVEIKTLDELKNALK |
| SEQ ID NO: | KQNVSSLDEKNSVSVDLPGGMKVLVSKEKDKDGKYSLMATVEKLELKGTSDK |
| 5: | NNGSGTLEGEKTDKSKVKLTIAEDLSKTTFEIFKEDGKTLVSKKVTLKDKSSTE |
| OspA ST5 | EKFNEKGEISEKTIVRANGTRLEYTDIKSDGSGKAKEVLKDFTLEGTLAADGKT |
| Native | TLKVTEGTVVLSKNILKSGEITVALDDSDTTQATKKTGKWDSKTSTLTISVNSQ |
| | KTKNLVFTKEDTITVQKYDSAGTNLEGKAVEITTLEKLKDALK |
| SEQ ID NO: | KQNVSSLDEKNSVSVDLPGGMTVLVSKEKDKDGKYSLEATVDKLELKGTSDK |
| 6: | NNGSGTLEGEKTDKSKVKSTIADDLSQTKFEIFKEDGKTLVSKKVTLKDKSST |
| OspA ST6 | EEKFNGKGETSEKTIVRANGTRLEYTDIKSDGSGKAKEVLKDFTLEGTLAADG |
| Native | KTTLKVTEGTVVLSKNILKSGEITAALDDSDTTRATKKTGKWDSKTSTLTISVN |
| | SQKTKNLVFTKEDTITVQRYDSAGTNLEGKAVEITTLKELKNALK |
| SEQ ID NO: | KQNVSSLDEKNSVSVDLPGEMKVLVSKEKDKDGKYSLEATVDKLELKGTSDK |
| 7: | NNGSGVLEGVKAAKSKAKLTIADDLSQTKFEIFKEDGKTLVSKKVTLKDKSSTE |
| OspA ST7 | EKFNDKGKLSEKVVTRANGTRLEYTEIQNDGSGKAKEVLKSLTLEGTLTADGE |
| Native | TKLTVEAGTVTLSKNISESGEITVELKDTETTPADKKSGTWDSKTSTLTISKNS |
| | QKTKQLVFTKENTITVQKYNTAGTKLEGSPAEIKDLEALKAALK |
| SEQ ID NO: | ANNQAGQKSSGSTQATTPNLTFE |
| 8 | |
| Linker 1 | |
| SEQ ID NO: | GTSDKNNGSGSKEKNKDGKYS |
| 9 | |
| Linker 2 | |

TABLE 4
mRNA sequences for OspA, 5′ UTR and 3′ UTR

| SEQ ID NO/ | |
|---|---|
| Description | Nucleotide sequence |
| SEQ ID NO: | GGACAGAUCGCCUGGAGACGCCAUCCACGCUGUUUUGACCUCCAUAGA |
| 10: | AGACACCGGGACCGAUCCAGCCUCCGCGGCCGGGAACGGUGCAUUGG |
| OspA ST1 | AACGCGGAUUCCCCGUGCCAAGAGUGACUCACCGUCCUUGACACGAUG |
| Native | AAGCAAAAUGUGAGUAGUCUGGACGAGAAAAAUAGCGUGAGUGUGGAU |
| | CUGCCAGGAGAGAUGAAGGUGCUGGUGUCUAAAGAGAAAAACAAGGAC |
| | GGAAAGUACGACCUGAUCGCCACUGUGGACAAGCUGGAGCUGAAAGGC |
| | ACCUCUGAUAAGAACAAUGGCUCUGGCGUGCUGGAAGGGGUGAAGGCA |
| | GACAAGAGCAAAGUGAAGCUGACUAUCUCUGACGACCUGGGGCAAACC |
| | ACACUGGAGGUCUUCAAGGAAGACGGCAAAACACUGGUCUCUAAGAAA |
| | GUCACCAGCAAGGAUAAGUCCUCCACAGAGGAGAAAUUCAACGAGAAA |
| | GGCGAAGUGUCUGAGAAGAUCAUCACCAGGGCCGACGGAACCAGGCUG |
| | GAGUACACCGGCAUCAAAAGCGACGGGUCAGGGAAGGCUAAGGAAGUG |
| | CUGAAAGGAUACGAUCUGAAAGGAGAGCUGUCCUCCGAGAAGACAACU |
| | CUGGUGGUGAAAGAGGGAACCGUUACACUGUCUAAGAACAUCUCCAAG |
| | UCUGGGGAAGUCAGCGUGGAGCUGAACGACACCGACAGCUCUGCUGC |
| | UACAAAGAAGACUGCUGCUUGGAACAGCGGCACCUCUACCCUGACAAU |
| | CACCGUGAAUUCUAAGAAGACCAAGGACCUGGUGUUCACUAAGGAGAA |
| | UACCAUUACUGUCCAGCAGUAUGAUUCCAAUGGGACUAAACUGGAAGG |
| | AUCCGCCGUGGAGAUUACUAAGCUGGACGAGAUCAAGAAUGCCCUGAA |
| | GUGACGGGUGGCAUCCCUGUGACCCCUCCCCAGUGCCUCUCCUGGCC |
| | CUGGAAGUUGCCACUCCAGUGCCCACCAGCCUUGUCCUAAUAAAAUUA |
| | AGUUGCAUCAAGCU |
| SEQ ID NO: | GGACAGAUCGCCUGGAGACGCCAUCCACGCUGUUUUGACCUCCAUAGA |
| 11: | AGACACCGGGACCGAUCCAGCCUCCGCGGCCGGGAACGGUGCAUUGG |
| OspA ST1 | AACGCGGAUUCCCCGUGCCAAGAGUGACUCACCGUCCUUGACACGAUG |
| Native | AAGCAAAACGUGUCCAGCCUGGACGAGAAGAAUUCCGUUAGCGUGGAU |
| | CUGCCAGGGGAGAUGAAAGUGCUGGUGUCCAAGGAAAAGAACAAAGAU |
| | GGCAAGUAUGACCUGAUUGCAACUGUGGACAAACUGGAGCUGAAGGGC |
| | ACUUCUGAUAAAAAUAACGGCAGCGGCGUUCUGGAGGGCGUUAAGGCC |
| | GACAAGUCCAAGGUGAAGCUGACAAUCUCCGACGACCUGGGGCAGACC |
| | ACACUGGAGGUGUUUAAAGAGGAUGGCAAGACCCUGGUCUCUAAAAAA |
| | GUGACUUCCAAAGACAAGUCCAGCACAGAAGAGAAAUUCAAUGAAAAGG |
| | GAGAGGUGUCCGAGAAGAUCAUCACUCGGGCCGACGGGACUAGGCUG |
| | GAGUACACCGGGAUCAAGAGCGACGGCUCCGGAAAGGCAAAGGAGGUG |
| | CUGAAAGGCUAUGACCUGAAAGGCGAGCUGAGCAGCGAGAAAACUACC |
| | CUGGUGGUGAAGGAAGGCACAGUGACUCUGUCAAAAAAUAUCUCUAAG |
| | UCCGGCGAGGUGAGCGUGGAACUGAAUGACACUGACAGCUCCGCUGC |
| | AACUAAGAAAACAGCCGCUUGGAACUCUGGCACAUCUACUCUCACUAUU |
| | ACCGUGAACUCCAAGAAAACCAAGGACCUGGUGUUUACCAAGGAGAAU |
| | ACAAUCACAGUGCAACAGUACGACAGCAACGGCACUAAGCUGGAGGGC |
| | UCCGCCGUUGAGAUUACUAAGCUCGACGAGAUCAAAAACGCCCUGAAA |
| | UGACGGGUGGCAUCCCUGUGACCCCUCCCCAGUGCCUCUCCUGGCCC |
| | UGGAAGUUGCCACUCCAGUGCCCACCAGCCUUGUCCUAAUAAAAUUAA |
| | GUUGCAUCAAGCU |
| SEQ ID NO: | GGACAGAUCGCCUGGAGACGCCAUCCACGCUGUUUUGACCUCCAUAGA |
| 12: | AGACACCGGGACCGAUCCAGCCUCCGCGGCCGGGAACGGUGCAUUGG |
| OspA ST2 | AACGCGGAUUCCCCGUGCCAAGAGUGACUCACCGUCCUUGACACGAUG |
| Native | AAACAGAACGUGUCUUCCCUGGACGAGAAAAAUUCUGCAAGCGUGGAC |
| | CUGCCUGGCGAAAUGAAGGUGCUGGUGAGCAAGGAAAAAGACAAGGAC |
| | GGCAAGUAUUCUCUGAAGGCAACAGUGGAUAAGAUUGAACUGAAGGGA |
| | ACAUCUGACAAGGAUAAUGGCAGUGGGGUGCUGGAAGGGACUAAGGAC |
| | GACAAGUCUAAGGCCAAGCUGACCAUCGCAGAUGACCUGUCCAAGACA |
| | ACCUUUGAGCUUUUUAAAGAGGACGGCAAGACCCUGGUGAGCCGGAAA |
| | GUGUCUUCUAAGGACAAGACAUCUACUGACGAGAUGUUCAAUGAAAAA |
| | GGGGAGCUGAGCGCCAAGACAAUGACCAGAGAGAACGGAACAAAGCUG |
| | GAGUACACUGAGAUGAAGUCCGACGGGACCGGAAAAGCUAAGGAAGUC |
| | CUGAAGAAUUUCACUCUGGAGGGCAAGGUUGCCAACGAUAAGGUGACA |
| | CUGGAGGUGAAAGAGGGAACAGUGACCCUGUCCAAGGAAAUUGCCAAG |
| | AGUGGCGAAGUGACCGUGGCUCUGAACGAUACCAAUACAACUCAGGCC |
| | ACAAAGAAGACCGGAGCAUGGGACUCCAAAACAUCCACCCUGACCAUUA |
| | GCGUUAAUUCAAAGAAAACCACCCAGCUGGUGUUUACUAAGCAGGACA |
| | CCAUCACCGUGCAGAAGUAUGAUUCCGCAGGCACUAAUCUGGAAGGGA |
| | CAGCUGUGGAGAUCAAGACCCUGGAUGAGCUGAAAAAUGCCCUGAAAU |
| | GACGGGUGGCAUCCCUGUGACCCCUCCCCAGUGCCUCUCCUGGCCCU |
| | GGAAGUUGCCACUCCAGUGCCCACCAGCCUUGUCCUAAUAAAAUUAAG |
| | UUGCAUCAAGCU |
| SEQ ID NO: | GGACAGAUCGCCUGGAGACGCCAUCCACGCUGUUUUGACCUCCAUAGA |
| 13 | AGACACCGGGACCGAUCCAGCCUCCGCGGCCGGGAACGGUGCAUUGG |
| OspA ST2 | AACGCGGAUUCCCCGUGCCAAGAGUGACUCACCGUCCUUGACACGAUG |
| Native | AAGCAGAACGUGAGUAGCCUGGAUGAAAAGAAUAGCGCCUCUGUGGAC |
| | CUGCCCGGGGAGAUGAAGGUGCUGGUCUCUAAAGAGAAAGAUAAGGAU |
| | GGAAAAUACAGCCUGAAGGCAACAGUGGACAAAAUCGAGCUCAAAGGAA |
| | CAUCCGAUAAGGAUAACGGGAGCGGAGUCCUGGAGGGCACCAAAGACG |
| | ACAAGAGCAAGGCCAAGCUGACAAUCGCCGAUGAUCUGAGUAAGACAA |
| | CUUUCGAGCUCUUUAAGGAGGACGGCAAGACCCUCGUGAGCAGAAAGG |
| | UGAGCAGCAAAGACAAGACCUCUACCGACGAGAUGUUCAAUGAGAAGG |
| | GGGAACUCUCUGCUAAGACAAUGACACGGGAGAAUGGAACUAAACUGG |
| | AGUACACAGAAAUGAAGUCUGACGGCACUGGGAAGGCAAAAGAGGUGC |
| | UGAAGAAUUUCACACUGGAAGGGAAGGUGGCUAACGAUAAAGUGACAC |
| | UGGAGGUUAAGGAGGGAACCGUGACUCUGUCUAAGGAGAUUGCAAAAA |
| | GCGGAGAGGUGACAGUCGCCCUGAACGACACUAACACCACCCAGGCCA |
| | CUAAGAAGACAGGAGCCUGGGACAGUAAAACCUCCACACUGACCAUUU |
| | CCGUGAACAGCAAGAAGACAACCCAGCUGGUCUUCACUAAACAGGAUA |
| | CUAUCACAGUCCAGAAGUACGACUCCGCCGGAACCAAUCUGGAGGGAA |
| | CUGCCGUGGAGAUCAAAACCCUGGACGAGCUCAAAAACGCCCUGAAGU |
| | GACGGGUGGCAUCCCUGUGACCCCUCCCCAGUGCCUCUCCUGGCCCU |
| | GGAAGUUGCCACUCCAGUGCCCACCAGCCUUGUCCUAAUAAAAUUAAG |
| | UUGCAUCAAGCU |
| SEQ ID NO: | GGACAGAUCGCCUGGAGACGCCAUCCACGCUGUUUUGACCUCCAUAGA |
| 14: | AGACACCGGGACCGAUCCAGCCUCCGCGGCCGGGAACGGUGCAUUGG |
| 5′ UTR | AACGCGGAUUCCCCGUGCCAAGAGUGACUCACCGUCCUUGACACG |
| SEQ ID NO: | CGGGUGGCAUCCCUGUGACCCCUCCCCAGUGCCUCUCCUGGCCCUGG |
| 15: | AAGUUGCCACUCCAGUGCCCACCAGCCUUGUCCUAAUAAAAUUAAGUU |
| 3′ UTR | GCAUC |
| SEQ ID NO: | AUGAAGCAAAAUGUGAGUAGUCUGGACGAGAAAAAUAGCGUGAGUGUG |
| 16: | GAUCUGCCAGGAGAGAUGAAGGUGCUGGUGUCUAAAGAGAAAAACAAG |
| Coding | GACGGAAAGUACGACCUGAUCGCCACUGUGGACAAGCUGGAGCUGAAA |
| sequence for | GGCACCUCUGAUAAGAACAAUGGCUCUGGCGUGCUGGAAGGGGUGAA |
| OspA ST1 | GGCAGACAAGAGCAAAGUGAAGCUGACUAUCUCUGACGACCUGGGGCA |
| Native (ORF | AACCACACUGGAGGUCUUCAAGGAAGACGGCAAAACACUGGUCUCUAA |
| only) | GAAAGUCACCAGCAAGGAUAAGUCCUCCACAGAGGAGAAAUUCAACGA |
| | GAAAGGCGAAGUGUCUGAGAAGAUCAUCACCAGGGCCGACGGAACCAG |
| | GCUGGAGUACACCGGCAUCAAAAGCGACGGGUCAGGGAAGGCUAAGGA |
| | AGUGCUGAAAGGAUACGAUCUGAAAGGAGAGCUGUCCUCCGAGAAGAC |
| | AACUCUGGUGGUGAAAGAGGGAACCGUUACACUGUCUAAGAACAUCUC |
| | CAAGUCUGGGGAAGUCAGCGUGGAGCUGAACGACACCGACAGCUCUGC |
| | UGCUACAAAGAAGACUGCUGCUUGGAACAGCGGCACCUCUACCCUGAC |
| | AAUCACCGUGAAUUCUAAGAAGACCAAGGACCUGGUGUUCACUAAGGA |
| | GAAUACCAUUACUGUCCAGCAGUAUGAUUCCAAUGGGACUAAACUGGA |
| | AGGAUCCGCCGUGGAGAUUACUAAGCUGGACGAGAUCAAGAAUGCCCU |
| | GAAGUGA |
| SEQ ID NO: | AUGAAGCAAAACGUGUCCAGCCUGGACGAGAAGAAUUCCGUUAGCGUG |
| 17: | GAUCUGCCAGGGGAGAUGAAAGUGCUGGUGUCCAAGGAAAAGAACAAA |
| Coding | GAUGGCAAGUAUGACCUGAUUGCAACUGUGGACAAACUGGAGCUGAAG |
| sequence for | GGCACUUCUGAUAAAAAUAACGGCAGCGGCGUUCUGGAGGGCGUUAAG |
| OspA ST1 | GCCGACAAGUCCAAGGUGAAGCUGACAAUCUCCGACGACCUGGGGCAG |
| Native (ORF | ACCACACUGGAGGUGUUUAAAGAGGAUGGCAAGACCCUGGUCUCUAAA |
| only) | AAAGUGACUUCCAAAGACAAGUCCAGCACAGAAGAGAAAUUCAAUGAAA |
| | AGGGAGAGGUGUCCGAGAAGAUCAUCACUCGGGCCGACGGGACUAGG |
| | CUGGAGUACACCGGGAUCAAGAGCGACGGCUCCGGAAAGGCAAAGGAG |
| | GUGCUGAAAGGCUAUGACCUGAAAGGCGAGCUGAGCAGCGAGAAAACU |
| | ACCCUGGUGGUGAAGGAAGGCACAGUGACUCUGUCAAAAAAUAUCUCU |
| | AAGUCCGGCGAGGUGAGCGUGGAACUGAAUGACACUGACAGCUCCGCU |
| | GCAACUAAGAAAACAGCCGCUUGGAACUCUGGCACAUCUACUCUCACU |
| | AUUACCGUGAACUCCAAGAAAACCAAGGACCUGGUGUUUACCAAGGAG |
| | AAUACAAUCACAGUGCAACAGUACGACAGCAACGGCACUAAGCUGGAG |
| | GGCUCCGCCGUUGAGAUUACUAAGCUCGACGAGAUCAAAAACGCCCUG |
| | AAAUGA |
| SEQ ID NO: | AUGAAACAGAACGUGUCUUCCCUGGACGAGAAAAAUUCUGCAAGCGUG |
| 18: | GACCUGCCUGGCGAAAUGAAGGUGCUGGUGAGCAAGGAAAAAGACAAG |
| Coding | GACGGCAAGUAUUCUCUGAAGGCAACAGUGGAUAAGAUUGAACUGAAG |
| sequence for | GGAACAUCUGACAAGGAUAAUGGCAGUGGGGUGCUGGAAGGGACUAAG |
| OspA ST2 | GACGACAAGUCUAAGGCCAAGCUGACCAUCGCAGAUGACCUGUCCAAG |
| Native (ORF | ACAACCUUUGAGCUUUUUAAAGAGGACGGCAAGACCCUGGUGAGCCGG |
| only) | AAAGUGUCUUCUAAGGACAAGACAUCUACUGACGAGAUGUUCAAUGAAA |
| | AAGGGGAGCUGAGCGCCAAGACAAUGACCAGAGAGAACGGAACAAAGC |
| | UGGAGUACACUGAGAUGAAGUCCGACGGGACCGGAAAAGCUAAGGAAG |
| | UCCUGAAGAAUUUCACUCUGGAGGGCAAGGUUGCCAACGAUAAGGUGA |
| | CACUGGAGGUGAAAGAGGGAACAGUGACCCUGUCCAAGGAAAUUGCCA |
| | AGAGUGGCGAAGUGACCGUGGCUCUGAACGAUACCAAUACAACUCAGG |
| | CCACAAAGAAGACCGGAGCAUGGGACUCCAAAACAUCCACCCUGACCAU |
| | UAGCGUUAAUUCAAAGAAAACCACCCAGCUGGUGUUUACUAAGCAGGA |
| | CACCAUCACCGUGCAGAAGUAUGAUUCCGCAGGCACUAAUCUGGAAGG |
| | GACAGCUGUGGAGAUCAAGACCCUGGAUGAGCUGAAAAAUGCCCUGAA |
| | AUGA |
| SEQ ID NO: | AUGAAGCAGAACGUGAGUAGCCUGGAUGAAAAGAAUAGCGCCUCUGUG |
| 19: | GACCUGCCCGGGGAGAUGAAGGUGCUGGUCUCUAAAGAGAAAGAUAAG |
| Coding | GAUGGAAAAUACAGCCUGAAGGCAACAGUGGACAAAAUCGAGCUCAAA |
| sequence for | GGAACAUCCGAUAAGGAUAACGGGAGCGGAGUCCUGGAGGGCACCAAA |
| OspA ST2 | GACGACAAGAGCAAGGCCAAGCUGACAAUCGCCGAUGAUCUGAGUAAG |
| Native (ORF | ACAACUUUCGAGCUCUUUAAGGAGGACGGCAAGACCCUCGUGAGCAGA |
| only) | AAGGUGAGCAGCAAAGACAAGACCUCUACCGACGAGAUGUUCAAUGAG |
| | AAGGGGGAACUCUCUGCUAAGACAAUGACACGGGAGAAUGGAACUAAA |
| | CUGGAGUACACAGAAAUGAAGUCUGACGGCACUGGGAAGGCAAAAGAG |
| | GUGCUGAAGAAUUUCACACUGGAAGGGAAGGUGGCUAACGAUAAAGUG |
| | ACACUGGAGGUUAAGGAGGGAACCGUGACUCUGUCUAAGGAGAUUGCA |
| | AAAAGCGGAGAGGUGACAGUCGCCCUGAACGACACUAACACCACCCAG |
| | GCCACUAAGAAGACAGGAGCCUGGGACAGUAAAACCUCCACACUGACC |
| | AUUUCCGUGAACAGCAAGAAGACAACCCAGCUGGUCUUCACUAAACAG |
| | GAUACUAUCACAGUCCAGAAGUACGACUCCGCCGGAACCAAUCUGGAG |
| | GGAACUGCCGUGGAGAUCAAAACCCUGGACGAGCUCAAAAACGCCCUG |
| | AAGUGA |

### Example 2: Immunogenicity of mRNA Encoding OspA Protein in Mice

The relative immunogenicity of the various OspA-expressing mRNA was tested in mice by measuring IgG titers against OspA, as described above in Example 1. Each mRNA was encapsulated into an LNP composed of 40% cationic lipid cKK-E10, 30% phospholipid DOPE, 1.5% PEGylated lipid DMGPEG2000, and 28.5% cholesterol. Alternatively, the LNP lipids may be recited as ratios where cationic lipid:PEGylated lipid:cholesterol:phospholipid is 40:1.5:28.5:30.

Each LNP-mRNA composition was administered to mice at a dose of 0.2 μg, 1 μg, 5 μg, or 10 μg. In total, 4 groups with 8 mice/group were used.

Three benchmark compositions were used: an OspA fusion with an AIOOH adjuvant (“OspA fusion ST1-ST2”) (at a 2 μg (1 μg by serotype)/dose); Lyme dog vaccine RECOMBITEK® (Merial) (at a 1 μg dose), and an OspA-ferritin fusion (ST1 or ST2) with an AF03 adjuvant (1.7 μg (of which 1 μg OspA+0.7 μg ferritin)/dose). The OspA-ferritin fusion is further described in US20210017238A1, incorporated herein by reference.

As a negative control, an mRNA-free LNP was administered to mice.

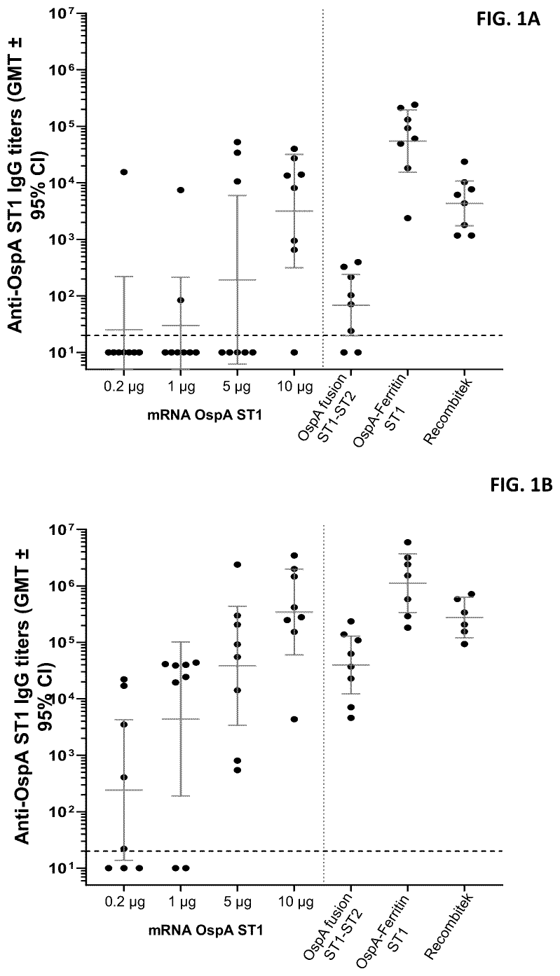

As shown in FIG. 1A, anti-OspA ST1 IgG titers were elevated post-dose 1 (day 20).

As shown in FIG. 1B, anti-OspA ST1 IgG titers were elevated further post-dose 2 (day 35).

The LNP alone negative control induced non-specific IgG titers that remained low (not shown).

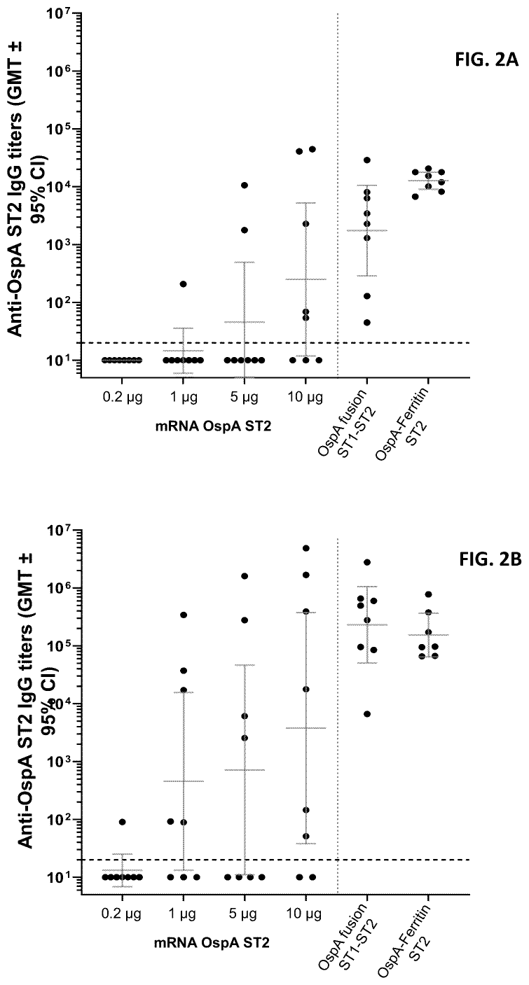

As shown in FIG. 2A, anti-OspA ST2 IgG titers were elevated post-dose 1 (day 20).

As shown in FIG. 2B anti-OspA ST2 IgG titers were elevated further post-dose 2 (day 35).

### Conclusions on Mouse Study:

mRNA coding for OspA ST1 and ST2 was immunogenic and induced strong anti-OspA IgG titers in mice both post-dose 1 and post-dose 2.

A dose effect was observed (i.e., increasing IgG titers with increasing dose). mRNA coding for OspA ST1 induced higher homologous IgG titers than mRNA coding for OspA ST2.

IgG titers induced by the mRNA-OspA ST1 were equivalent or higher than those induced by the benchmark OspA fusion ST1-ST2 and Recombitek (ST1).

Other embodiments of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

All patents and publications cited herein are incorporated by reference herein in their entirety.

1. A Lyme disease vaccine, comprising a messenger RNA (mRNA) comprising an open reading frame (ORF) encoding at least one antigenic polypeptide derived from at least one bacteria of the genus Borrelia.

2. The Lyme disease vaccine of claim 1, wherein the mRNA is formulated in a lipid nanoparticle (LNP).

3. The Lyme disease vaccine of claim 1, wherein the mRNA is non-replicating mRNA.

4. (canceled)

5. The Lyme disease vaccine of claim 1, wherein the bacteria of the genus Borrelia is selected from the species B. burgdorferi, afzelii, garinii, bavariensis, mayonii, spielmanii, lusitaniae, bissettii and/or valaisiana, or any strain or isolate thereof.

6. The Lyme disease vaccine of claim 1, wherein the at least one antigenic polypeptide comprises at least one lipoprotein of Borrelia.

7. The Lyme disease vaccine of claim 1, wherein:
the at least one antigenic polypeptide or lipoprotein is OspA or a fragment or variant thereof;
the OspA or a fragment or variant thereof comprises at least 5 amino acids;
the OspA or a fragment or variant thereof is derived from OspA serotype (ST) 1, 2, 3, 4, 5, 6, and/or 7;
the OspA or a fragment or variant thereof is derived from Borrelia burgdorferi strain B31 of Serotype 1, Borrelia afzelii strain PKO of Serotype 2, Borrelia garinii strain PBr of Serotype 3, Borrelia bavariensis of Serotype 4, Borrelia garinii of Serotype 5, Borrelia garinii of serotype 6, or Borrelia garinii of Serotype 7; and/or
the at least one OspA polypeptide comprises an amino acid sequence with at least 85% identity to any one of SEQ ID NOs: 1-7.

8-11. (canceled)

12. The Lyme disease vaccine of claim 1, wherein the mRNA comprises a nucleotide sequence that is at least 85% identical to any one of SEQ ID NOs: 10-13 and 16-19.

13. The Lyme disease vaccine of claim 1, wherein:
the mRNA encodes at least two different OspA serotypes or fragments or variants thereof;
the OspA of one serotype or fragment or variant thereof, preferably comprising at least 5 amino acids, is fused to a OspA of a different serotype, or fragment or variant thereof, preferably comprising at least 5 amino acids; and/or
the fused OspA of different serotypes or fragments or variants thereof are separated by a linker sequence, optionally wherein the linker sequence is derived from P66 or comprises an amino acid sequence with at least 85% identity to SEQ ID NO: 8 or to SEQ ID NO: 9.

14-16. (canceled)

17. The Lyme disease vaccine of claim 1, wherein the mRNA comprises at least one chemical modification, optionally wherein
the chemical modification is selected from the group consisting of pseudouridine, N1-methylpseudouridine, 2-thiouridine, 4′-thiouridine, 5-methylcytidine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine;
the chemical modification is selected from the group consisting of pseudouridine, N1-methylpseudouridine, 5-methylcytidine, 5-methoxyuridine, and a combination thereof; and/or
the chemical modification is N1-methylpseudouridine.

18-20. (canceled)

21. The Lyme disease vaccine of claim 1, wherein the mRNA is formulated in a lipid nanoparticle (LNP) optionally wherein
the LNP comprises at least one cationic lipid;
the cationic lipid is biodegradable or is not biodegradable;
the cationic lipid is cleavable or the cationic lipid is not cleavable;
the cationic lipid is selected from the group consisting of ML7/OF-02; cKK-E10; GL-HEPES-E3-E10-DS-3-E18-1; GL-HEPES-E3-E12-DS-4-E10; GL-HEPES-E3-E12-DS-3-E14; 9-heptadecanyl 8-{(2-hydroxyethyl)[6-oxo-6-(undecyloxy)hexyl]amino}octanoate (SM-102); and (4-hydroxybutyl)azanediyl]di(hexane-6,1-diyl) bis(2-hexyldecanoate) (ALC-0315); and/or
the cationic lipid is cKK-E10.

22-28. (canceled)

29. The Lyme disease vaccine of claim 21, wherein the LNP further comprises:
a polyethylene glycol (PEG) conjugated (PEGylated) lipid optionally wherein the PEGylated lipid comprises dimyristoyl-PEG2000 (DMG-PEG2000) or 2-[(polyethylene glycol)-2000]-N,N-ditetradecylacetamide (ALC-0159);
a cholesterol-based lipid optionally wherein the cholesterol-based lipid comprises cholesterol; and/or
a helper lipid optionally wherein the helper lipid comprises 1,2-dioleoyl-SN-glycero-3-phosphoethanolamine (DOPE) or 1,2-distearoyl-sn-glycero-3-phosphocholine (DSPC).

30-34. (canceled)

35. The Lyme disease vaccine of claim 21, wherein the LNP comprises:
a cationic lipid at a molar ratio of 35% to 55%;
a polyethylene glycol (PEG) conjugated (PEGylated) lipid at a molar ratio of 0.25% to 2.75%,
a cholesterol-based lipid at a molar ratio of 20% to 45%, and
a helper lipid at a molar ratio of 5% to 35%,
wherein all of the molar ratios are relative to the total lipid content of the LNP.

36. The Lyme disease vaccine of claim 35, wherein the LNP comprises:
a cationic lipid at a molar ratio of 40%,
a PEGylated lipid at a molar ratio of 1.5%,
a cholesterol-based lipid at a molar ratio of 28.5%, and
a helper lipid at a molar ratio of 30%.

37. The Lyme disease vaccine of claim 35, wherein the LNP comprises:
cKK-E10 at a molar ratio of 40%,
DMG-PEG2000 at a molar ratio of 1.5%,
cholesterol at a molar ratio of 28.5%, and
DOPE at a molar ratio of 30%.

38. The Lyme disease vaccine of claim 21, wherein the LNP has an average diameter of 30-200 nm optionally wherein the LNP has an average diameter of 80-150 nm.

39. (canceled)

40. The Lyme disease vaccine of claim 1, wherein the mRNA comprises at least one 5′ cap, at least one 5′ untranslated region (5′ UTR), at least one 3′ untranslated region (3′ UTR), and/or at least one polyadenylation (polyA) sequence.

41. (canceled)

42. A Lyme disease vaccine, comprising a messenger RNA (mRNA), wherein the mRNA comprises at least of the following structural elements:
(i) a 5′ cap with the following structure: 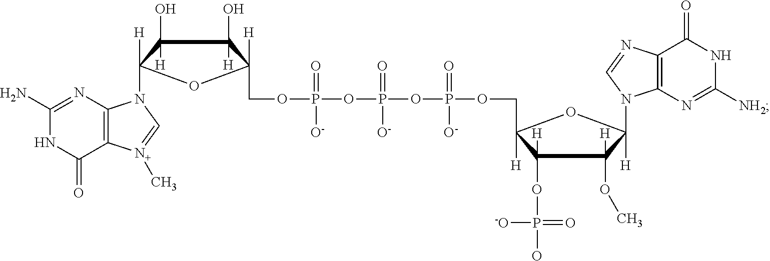
(ii) a 5′ untranslated region (5′ UTR) having the nucleic acid sequence of SEQ ID NO: 14;
(iii) at least one OspA open reading frame having any one of the nucleic acid sequences of SEQ ID NOs: 16-19;
(iv) a 3′ untranslated region (3′ UTR) having the nucleic acid sequence of SEQ ID NO: 15; and
(v) a polyA tail.

43. A Lyme disease vaccine, comprising a messenger RNA (mRNA) comprising an open reading frame (ORF) encoding at least one antigenic polypeptide derived from at least one bacteria of the genus Borrelia, wherein the mRNA comprises at least the following structural elements:
(i) a 5′ cap with the following structure: 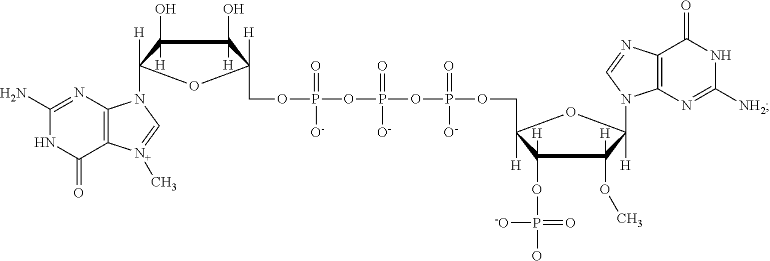
(ii) a 5′ untranslated region (5′ UTR) having the nucleic acid sequence of SEQ ID NO: 14;
(iii) at least one OspA open reading frame having any one of the nucleic acid sequences of SEQ ID NOs: 16-19;
(iv) a 3′ untranslated region (3′ UTR) having the nucleic acid sequence of SEQ ID NO: 15; and
(v) a polyA tail;
wherein the mRNA is formulated in a lipid nanoparticle (LNP) comprising:
cKK-E10 at a molar ratio of 40%,
DMG-PEG2000 at a molar ratio of 1.5%,
cholesterol at a molar ratio of 28.5%, and
DOPE at a molar ratio of 30%.

44. A method of eliciting an immune response in a subject, comprising administering to the subject the Lyme disease vaccine of claim 1, optionally wherein the immune response is a humoral immune response.

45. (canceled)

46. A method of treating or preventing Lyme disease in a subject in need thereof, comprising administering to the subject the Lyme disease vaccine of claim 1; optionally wherein:
the subject has a higher serum concentration of antibodies against OspA after administration of the Lyme disease vaccine, relative to a subject that is administered a Lyme disease vaccine comprising an OspA recombinant protein vaccine;
the subject is a mammal; and/or
the subject is a human, a dog, a cat, a llama, a bovine, a sheep, a goat, a horse, a rodent, a mouse, a rat, a rabbit, a monkey, a primate or a pig, more preferably a human.

47-49. (canceled)


SEQUENCE LISTING (21 sequences)

SEQ ID NO: 1 - protein, 256 aa, synthetic construct
  REGION 1..256: note = OspA ST1 Native
KQNVSSLDEKNSVSVDLPGEMKVLVSKEKNKDGKYDLIATVDKLELKGTSDKNNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGYDLKGELSSEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALK

SEQ ID NO: 2 - protein, 256 aa, synthetic construct
  REGION 1..256: note = OspA ST2 Native
KQNVSSLDEKNSASVDLPGEMKVLVSKEKDKDGKYSLKATVDKIELKGTSDKDNGSGVLEGTKDDKSKAKLTIADDLSKTTFELFKEDGKTLVSRKVSSKDKTSTDEMFNEKGELSAKTMTRENGTKLEYTEMKSDGTGKAKEVLKNFTLEGKVANDKVTLEVKEGTVTLSKEIAKSGEVTVALNDTNTTQATKKTGAWDSKTSTLTISVNSKKTTQLVFTKQDTITVQKYDSAGTNLEGTAVEIKTLDELKNALK

SEQ ID NO: 3 - protein, 257 aa, synthetic construct
  REGION 1..257: note = OspA ST3 Native
KQNVSSLDEKNSVSVDLPGGMKVLVSKEKDKDGKYSLMATVEKLELKGTSDKSNGSGVLEGEKADKSKAKLTISQDLNQTTFEIFKEDGKTLVSRKVNSKDKSSTEEKFNDKGKLSEKVVTRANGTRLEYTEIKNDGSGKAKEVLKGFALEGTLTDGGETKLTVTEGTVTLSKNISKSGEITVALNDTETTPADKKTGEWKSDTSTLTISKNSQKPKQLVFTKENTITVQNYNRAGNALEGSPAEIKDLAELKAALK

SEQ ID NO: 4 - protein, 256 aa, synthetic construct
  REGION 1..256: note = OspA ST4 Native
KQNVSSLDEKNSVSVDLPGEMKVLVSKEKDKDGKYSLMATVDKLELKGTSDKSNGSGTLEGEKSDKSKAKLTISEDLSKTTFEIFKEDGKTLVSKKVNSKDKSSIEEKFNAKGELSEKTILRANGTRLEYTEIKSDGTGKAKEVLKDFALEGTLAADKTTLKVTEGTVVLSKHIPNSGEITVELNDSNSTQATKKTGKWDSNTSTLTISVNSKKTKNIVFTKEDTITVQKYDSAGTNLEGNAVEIKTLDELKNALK

SEQ ID NO: 5 - protein, 257 aa, synthetic construct
  REGION 1..257: note = OspA ST5 Native
KQNVSSLDEKNSVSVDLPGGMKVLVSKEKDKDGKYSLMATVEKLELKGTSDKNNGSGTLEGEKTDKSKVKLTIAEDLSKTTFEIFKEDGKTLVSKKVTLKDKSSTEEKFNEKGEISEKTIVRANGTRLEYTDIKSDGSGKAKEVLKDFTLEGTLAADGKTTLKVTEGTVVLSKNILKSGEITVALDDSDTTQATKKTGKWDSKTSTLTISVNSQKTKNLVFTKEDTITVQKYDSAGTNLEGKAVEITTLEKLKDALK

SEQ ID NO: 6 - protein, 257 aa, synthetic construct
  REGION 1..257: note = OspA ST6 Native
KQNVSSLDEKNSVSVDLPGGMTVLVSKEKDKDGKYSLEATVDKLELKGTSDKNNGSGTLEGEKTDKSKVKSTIADDLSQTKFEIFKEDGKTLVSKKVTLKDKSSTEEKFNGKGETSEKTIVRANGTRLEYTDIKSDGSGKAKEVLKDFTLEGTLAADGKTTLKVTEGTVVLSKNILKSGEITAALDDSDTTRATKKTGKWDSKTSTLTISVNSQKTKNLVFTKEDTITVQRYDSAGTNLEGKAVEITTLKELKNALK

SEQ ID NO: 7 - protein, 257 aa, synthetic construct
  REGION 1..257: note = OspA ST7 Native
KQNVSSLDEKNSVSVDLPGEMKVLVSKEKDKDGKYSLEATVDKLELKGTSDKNNGSGVLEGVKAAKSKAKLTIADDLSQTKFEIFKEDGKTLVSKKVTLKDKSSTEEKFNDKGKLSEKVVTRANGTRLEYTEIQNDGSGKAKEVLKSLTLEGTLTADGETKLTVEAGTVTLSKNISESGEITVELKDTETTPADKKSGTWDSKTSTLTISKNSQKTKQLVFTKENTITVQKYNTAGTKLEGSPAEIKDLEALKAALK

SEQ ID NO: 8 - protein, 23 aa, synthetic construct
  REGION 1..23: note = Linker 1
ANNQAGQKSSGSTQATTPNLTFE

SEQ ID NO: 9 - protein, 21 aa, synthetic construct
  REGION 1..21: note = Linker 2
GTSDKNNGSGSKEKNKDGKYS

SEQ ID NO: 10 - RNA, 1019 nt, synthetic construct
  misc_feature 1..1019: note = OspA ST1 Native
  source 1..1019: mol_type = other RNA
ggacagatcgcctggagacgccatccacgctgttttgacctccatagaagacaccgggaccgatccagcctccgcggccgggaacggtgcattggaacgcggattccccgtgccaagagtgactcaccgtccttgacacgatgaagcaaaatgtgagtagtctggacgagaaaaatagcgtgagtgtggatctgccaggagagatgaaggtgctggtgtctaaagagaaaaacaaggacggaaagtacgacctgatcgccactgtggacaagctggagctgaaaggcacctctgataagaacaatggctctggcgtgctggaaggggtgaaggcagacaagagcaaagtgaagctgactatctctgacgacctggggcaaaccacactggaggtcttcaaggaagacggcaaaacactggtctctaagaaagtcaccagcaaggataagtcctccacagaggagaaattcaacgagaaaggcgaagtgtctgagaagatcatcaccagggccgacggaaccaggctggagtacaccggcatcaaaagcgacgggtcagggaaggctaaggaagtgctgaaaggatacgatctgaaaggagagctgtcctccgagaagacaactctggtggtgaaagagggaaccgttacactgtctaagaacatctccaagtctggggaagtcagcgtggagctgaacgacaccgacagctctgctgctacaaagaagactgctgcttggaacagcggcacctctaccctgacaatcaccgtgaattctaagaagaccaaggacctggtgttcactaaggagaataccattactgtccagcagtatgattccaatgggactaaactggaaggatccgccgtggagattactaagctggacgagatcaagaatgccctgaagtgacgggtggcatccctgtgacccctccccagtgcctctcctggccctggaagttgccactccagtgcccaccagccttgtcctaataaaattaagttgcatcaagct

SEQ ID NO: 11 - RNA, 1019 nt, synthetic construct
  misc_feature 1..1019: note = OspA ST1 Native
  source 1..1019: mol_type = other RNA
ggacagatcgcctggagacgccatccacgctgttttgacctccatagaagacaccgggaccgatccagcctccgcggccgggaacggtgcattggaacgcggattccccgtgccaagagtgactcaccgtccttgacacgatgaagcaaaacgtgtccagcctggacgagaagaattccgttagcgtggatctgccaggggagatgaaagtgctggtgtccaaggaaaagaacaaagatggcaagtatgacctgattgcaactgtggacaaactggagctgaagggcacttctgataaaaataacggcagcggcgttctggagggcgttaaggccgacaagtccaaggtgaagctgacaatctccgacgacctggggcagaccacactggaggtgtttaaagaggatggcaagaccctggtctctaaaaaagtgacttccaaagacaagtccagcacagaagagaaattcaatgaaaagggagaggtgtccgagaagatcatcactcgggccgacgggactaggctggagtacaccgggatcaagagcgacggctccggaaaggcaaaggaggtgctgaaaggctatgacctgaaaggcgagctgagcagcgagaaaactaccctggtggtgaaggaaggcacagtgactctgtcaaaaaatatctctaagtccggcgaggtgagcgtggaactgaatgacactgacagctccgctgcaactaagaaaacagccgcttggaactctggcacatctactctcactattaccgtgaactccaagaaaaccaaggacctggtgtttaccaaggagaatacaatcacagtgcaacagtacgacagcaacggcactaagctggagggctccgccgttgagattactaagctcgacgagatcaaaaacgccctgaaatgacgggtggcatccctgtgacccctccccagtgcctctcctggccctggaagttgccactccagtgcccaccagccttgtcctaataaaattaagttgcatcaagct

SEQ ID NO: 12 - RNA, 1019 nt, synthetic construct
  misc_feature 1..1019: note = OspA ST2 Native
  source 1..1019: mol_type = other RNA
ggacagatcgcctggagacgccatccacgctgttttgacctccatagaagacaccgggaccgatccagcctccgcggccgggaacggtgcattggaacgcggattccccgtgccaagagtgactcaccgtccttgacacgatgaaacagaacgtgtcttccctggacgagaaaaattctgcaagcgtggacctgcctggcgaaatgaaggtgctggtgagcaaggaaaaagacaaggacggcaagtattctctgaaggcaacagtggataagattgaactgaagggaacatctgacaaggataatggcagtggggtgctggaagggactaaggacgacaagtctaaggccaagctgaccatcgcagatgacctgtccaagacaacctttgagctttttaaagaggacggcaagaccctggtgagccggaaagtgtcttctaaggacaagacatctactgacgagatgttcaatgaaaaaggggagctgagcgccaagacaatgaccagagagaacggaacaaagctggagtacactgagatgaagtccgacgggaccggaaaagctaaggaagtcctgaagaatttcactctggagggcaaggttgccaacgataaggtgacactggaggtgaaagagggaacagtgaccctgtccaaggaaattgccaagagtggcgaagtgaccgtggctctgaacgataccaatacaactcaggccacaaagaagaccggagcatgggactccaaaacatccaccctgaccattagcgttaattcaaagaaaaccacccagctggtgtttactaagcaggacaccatcaccgtgcagaagtatgattccgcaggcactaatctggaagggacagctgtggagatcaagaccctggatgagctgaaaaatgccctgaaatgacgggtggcatccctgtgacccctccccagtgcctctcctggccctggaagttgccactccagtgcccaccagccttgtcctaataaaattaagttgcatcaagct

SEQ ID NO: 13 - RNA, 1019 nt, synthetic construct
  misc_feature 1..1019: note = OspA ST2 Native
  source 1..1019: mol_type = other RNA
ggacagatcgcctggagacgccatccacgctgttttgacctccatagaagacaccgggaccgatccagcctccgcggccgggaacggtgcattggaacgcggattccccgtgccaagagtgactcaccgtccttgacacgatgaagcagaacgtgagtagcctggatgaaaagaatagcgcctctgtggacctgcccggggagatgaaggtgctggtctctaaagagaaagataaggatggaaaatacagcctgaaggcaacagtggacaaaatcgagctcaaaggaacatccgataaggataacgggagcggagtcctggagggcaccaaagacgacaagagcaaggccaagctgacaatcgccgatgatctgagtaagacaactttcgagctctttaaggaggacggcaagaccctcgtgagcagaaaggtgagcagcaaagacaagacctctaccgacgagatgttcaatgagaagggggaactctctgctaagacaatgacacgggagaatggaactaaactggagtacacagaaatgaagtctgacggcactgggaaggcaaaagaggtgctgaagaatttcacactggaagggaaggtggctaacgataaagtgacactggaggttaaggagggaaccgtgactctgtctaaggagattgcaaaaagcggagaggtgacagtcgccctgaacgacactaacaccacccaggccactaagaagacaggagcctgggacagtaaaacctccacactgaccatttccgtgaacagcaagaagacaacccagctggtcttcactaaacaggatactatcacagtccagaagtacgactccgccggaaccaatctggagggaactgccgtggagatcaaaaccctggacgagctcaaaaacgccctgaagtgacgggtggcatccctgtgacccctccccagtgcctctcctggccctggaagttgccactccagtgcccaccagccttgtcctaataaaattaagttgcatcaagct

SEQ ID NO: 14 - RNA, 140 nt, synthetic construct
  misc_feature 1..140: note = 5' UTR
  source 1..140: mol_type = other RNA
ggacagatcgcctggagacgccatccacgctgttttgacctccatagaagacaccgggaccgatccagcctccgcggccgggaacggtgcattggaacgcggattccccgtgccaagagtgactcaccgtccttgacacg

SEQ ID NO: 15 - RNA, 100 nt, synthetic construct
  misc_feature 1..100: note = 3' UTR
  source 1..100: mol_type = other RNA
cgggtggcatccctgtgacccctccccagtgcctctcctggccctggaagttgccactccagtgcccaccagccttgtcctaataaaattaagttgcatc

SEQ ID NO: 16 - RNA, 774 nt, synthetic construct
  misc_feature 1..774: note = Coding sequence for OspA ST1 Native
  source 1..774: mol_type = other RNA
atgaagcaaaatgtgagtagtctggacgagaaaaatagcgtgagtgtggatctgccaggagagatgaaggtgctggtgtctaaagagaaaaacaaggacggaaagtacgacctgatcgccactgtggacaagctggagctgaaaggcacctctgataagaacaatggctctggcgtgctggaaggggtgaaggcagacaagagcaaagtgaagctgactatctctgacgacctggggcaaaccacactggaggtcttcaaggaagacggcaaaacactggtctctaagaaagtcaccagcaaggataagtcctccacagaggagaaattcaacgagaaaggcgaagtgtctgagaagatcatcaccagggccgacggaaccaggctggagtacaccggcatcaaaagcgacgggtcagggaaggctaaggaagtgctgaaaggatacgatctgaaaggagagctgtcctccgagaagacaactctggtggtgaaagagggaaccgttacactgtctaagaacatctccaagtctggggaagtcagcgtggagctgaacgacaccgacagctctgctgctacaaagaagactgctgcttggaacagcggcacctctaccctgacaatcaccgtgaattctaagaagaccaaggacctggtgttcactaaggagaataccattactgtccagcagtatgattccaatgggactaaactggaaggatccgccgtggagattactaagctggacgagatcaagaatgccctgaagtga

SEQ ID NO: 17 - RNA, 774 nt, synthetic construct
  misc_feature 1..774: note = Coding sequence for OspA ST1 Native
  source 1..774: mol_type = other RNA
atgaagcaaaacgtgtccagcctggacgagaagaattccgttagcgtggatctgccaggggagatgaaagtgctggtgtccaaggaaaagaacaaagatggcaagtatgacctgattgcaactgtggacaaactggagctgaagggcacttctgataaaaataacggcagcggcgttctggagggcgttaaggccgacaagtccaaggtgaagctgacaatctccgacgacctggggcagaccacactggaggtgtttaaagaggatggcaagaccctggtctctaaaaaagtgacttccaaagacaagtccagcacagaagagaaattcaatgaaaagggagaggtgtccgagaagatcatcactcgggccgacgggactaggctggagtacaccgggatcaagagcgacggctccggaaaggcaaaggaggtgctgaaaggctatgacctgaaaggcgagctgagcagcgagaaaactaccctggtggtgaaggaaggcacagtgactctgtcaaaaaatatctctaagtccggcgaggtgagcgtggaactgaatgacactgacagctccgctgcaactaagaaaacagccgcttggaactctggcacatctactctcactattaccgtgaactccaagaaaaccaaggacctggtgtttaccaaggagaatacaatcacagtgcaacagtacgacagcaacggcactaagctggagggctccgccgttgagattactaagctcgacgagatcaaaaacgccctgaaatga

SEQ ID NO: 18 - RNA, 774 nt, synthetic construct
  misc_feature 1..774: note = Coding sequence for OspA ST2 Native
  source 1..774: mol_type = other RNA
atgaaacagaacgtgtcttccctggacgagaaaaattctgcaagcgtggacctgcctggcgaaatgaaggtgctggtgagcaaggaaaaagacaaggacggcaagtattctctgaaggcaacagtggataagattgaactgaagggaacatctgacaaggataatggcagtggggtgctggaagggactaaggacgacaagtctaaggccaagctgaccatcgcagatgacctgtccaagacaacctttgagctttttaaagaggacggcaagaccctggtgagccggaaagtgtcttctaaggacaagacatctactgacgagatgttcaatgaaaaaggggagctgagcgccaagacaatgaccagagagaacggaacaaagctggagtacactgagatgaagtccgacgggaccggaaaagctaaggaagtcctgaagaatttcactctggagggcaaggttgccaacgataaggtgacactggaggtgaaagagggaacagtgaccctgtccaaggaaattgccaagagtggcgaagtgaccgtggctctgaacgataccaatacaactcaggccacaaagaagaccggagcatgggactccaaaacatccaccctgaccattagcgttaattcaaagaaaaccacccagctggtgtttactaagcaggacaccatcaccgtgcagaagtatgattccgcaggcactaatctggaagggacagctgtggagatcaagaccctggatgagctgaaaaatgccctgaaatga

SEQ ID NO: 19 - RNA, 774 nt, synthetic construct
  misc_feature 1..774: note = Coding sequence for OspA ST2 Native
  source 1..774: mol_type = other RNA
atgaagcagaacgtgagtagcctggatgaaaagaatagcgcctctgtggacctgcccggggagatgaaggtgctggtctctaaagagaaagataaggatggaaaatacagcctgaaggcaacagtggacaaaatcgagctcaaaggaacatccgataaggataacgggagcggagtcctggagggcaccaaagacgacaagagcaaggccaagctgacaatcgccgatgatctgagtaagacaactttcgagctctttaaggaggacggcaagaccctcgtgagcagaaaggtgagcagcaaagacaagacctctaccgacgagatgttcaatgagaagggggaactctctgctaagacaatgacacgggagaatggaactaaactggagtacacagaaatgaagtctgacggcactgggaaggcaaaagaggtgctgaagaatttcacactggaagggaaggtggctaacgataaagtgacactggaggttaaggagggaaccgtgactctgtctaaggagattgcaaaaagcggagaggtgacagtcgccctgaacgacactaacaccacccaggccactaagaagacaggagcctgggacagtaaaacctccacactgaccatttccgtgaacagcaagaagacaacccagctggtcttcactaaacaggatactatcacagtccagaagtacgactccgccggaaccaatctggagggaactgccgtggagatcaaaaccctggacgagctcaaaaacgccctgaagtga

SEQ ID NO: 20 - RNA, 11 nt, synthetic construct
  source 1..11: mol_type = other RNA
gggatcctacc

SEQ ID NO: 21 - RNA, 110 nt, synthetic construct
  source 1..110: mol_type = other RNA
aaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa